(19) United States
(12) Patent Application Publication
(10) Pub. No.: US 2025/0011760 A1
(43) Pub. Date: Jan. 9, 2025

(54) LIBRARY PREPARATION METHODS AND COMPOSITIONS AND USES THEREFOR

(71) Applicant: LIFE TECHNOLOGIES CORPORATION, Carlsbad, CA (US)

(72) Inventors: Mark Andersen, Carlsbad, CA (US); Sihong Chen, Vista, CA (US); Daniel Mazur, San Diego, CA (US); Xinzhan Peng, San Diego, CA (US); Guobin Luo, Carlsbad, CA (US)

(21) Appl. No.: 18/768,211

(22) Filed: Jul. 10, 2024

Related U.S. Application Data
(62) Division of application No. 17/302,674, filed on May 10, 2021, now Pat. No. 12,065,643.
Application No. 17/302,674 is a continuation of application No. 16/024,175, filed on Jun. 29, 2018, now Pat. No. 11,001,836.
(60) Provisional application No. 62/685,424, filed on Jun. 15, 2018.
(60) Provisional application No. 62/614,362, filed on Jan. 6, 2018.
(60) Provisional application No. 62/527,893, filed on Jun. 30, 2017.

(51) Int. Cl. C12N 15/10; C12Q 1/6806; C12Q 1/6853; C12Q 1/6855; C12Q 1/686; C40B 20/04; C40B 40/06; C40B 50/06

(52) U.S. Cl. CPC C12N 15/1068; C12N 15/1065; C12Q 1/6806; C12Q 1/6853; C12Q 1/6855; C12Q 1/686; C40B 20/04; C40B 40/06; C40B 50/06

(57) ABSTRACT

Provided are methods for preparing a library of target nucleic acid sequences, as well as compositions and uses therefor. Methods comprise contacting a nucleic acid sample with a plurality of adaptors capable of amplification of one or more target nucleic acid sequences under conditions wherein the target nucleic acid(s) undergo a first amplification; digesting the resulting first amplification products; repairing the digested target amplicons; and amplifying the repaired products in a second amplification, thereby producing a library of target nucleic acid sequence. Each of the plurality of adaptor compositions comprise a handle and a targeted nucleic acid sequence and optionally one or more tag sequences. Provided methods may be carried out in a single, addition only workflow reaction, allowing for rapid production of highly multiplexed targeted libraries, optionally including unique tag sequences. Resulting library compositions are useful for a variety of applications, including sequencing applications.

### RELATED APPLICATIONS

This application is a divisional of U.S. Non-provisional application Ser. No. 17/302,674, filed May 10, 2021, which is a continuation of U.S. Non-provisional application Ser. No. 16/024,175, filed Jun. 29, 2018, now U.S. Pat. No. 11,001,836, which claims priority to and the benefit under 35 USC § 119 (e) of each of U.S. Provisional Application No. 62/527,893 filed Jun. 30, 2017, U.S. Provisional Application No. 62/614,362, filed Jan. 6, 2018, and U.S. Provisional Application No. 62/685,424, filed Jun. 15, 2018. The entire contents of each of the aforementioned applications are herein incorporated by reference in their entirety.

### SEQUENCE LISTING

This application hereby incorporates by reference the material of the electronic Sequence Listing filed concurrently herewith. The material in the electronic Sequence Listing is submitted as an Extensible Markup Language (.xml) file entitled “TP103456USDIVI_ST26V2.xml” created on Jul. 17, 2024, which has a file size of 3,741,499 bytes, and is herein incorporated by reference in its entirety.

### FIELD OF THE INVENTION

The present invention relates to methods of preparing a library of target nucleic acid sequences and compositions and uses therefor.

### BRIEF SUMMARY OF THE INVENTION

Provided are methods for preparing a library of target nucleic acid sequences, as well as compositions and uses therefor. Methods comprise contacting a nucleic acid sample with a plurality of adaptors capable of amplification of one or more target nucleic acid sequences under conditions wherein the target nucleic acid(s) undergo a first amplification; digesting the resulting first amplification products; repairing the digested target amplicons; and amplifying the repaired products in a second amplification, thereby producing a library of target nucleic acid sequence. Each of the plurality of adaptor compositions comprise a handle and a targeted nucleic acid sequence and optionally one or more tag sequences. Provided methods may be carried out in a single, addition only workflow reaction, allowing for rapid production of highly multiplexed targeted libraries, optionally including unique tag sequences. Resulting library compositions are useful for a variety of applications, including sequencing applications.

One aspect of the invention comprises methods for preparing a library of target nucleic acid sequences. In certain embodiments the methods comprise contacting a nucleic acid sample with a plurality of adaptors wherein each of a pair of adaptors are capable of amplification of one or more target nucleic acid sequences in the sample under conditions wherein the target nucleic acid(s) undergo a first amplification. The methods further comprise digesting the resulting first amplification products to reduce or eliminate any primer dimers resulting in the reaction and preparing partially digested amplicons, thereby preparing resulting gapped, double stranded partially digested amplicons. The methods further comprise repairing the partially digested target amplicons; then amplifying the repaired products in a second amplification using universal primers to thereby produce a library of target nucleic acid sequences. Each of the plurality of adaptors used in the provided methods comprise a 5′ universal handle sequence and a 3′ target nucleic acid sequence and a cleavable moiety. Two or more target specific adaptor pairs are included for use in provided methods, wherein each of the 3′ target specific sequences comprise cleavable moieties. Optionally, one or more tag sequences are included.

In another aspect of the invention methods for preparing a library of target nucleic acid sequences having unique tag sequences is provided. In certain embodiments the methods comprise contacting a nucleic acid sample with a plurality of adaptors wherein each of a pair of adaptors are capable of amplification of one or more target nucleic acid sequences in the sample under conditions wherein the target nucleic acid(s) undergo a first amplification. The methods further comprise digesting the resulting first amplification products to reduce or eliminate any primer dimers resulting in the reaction and preparing partially digested amplicons, thereby preparing resulting gapped, double stranded partially digested amplicons. The methods further comprise repairing the partially digested target amplicons; then amplifying the repaired products in a second amplification using universal primers to thereby produce a library of target nucleic acid sequences. Each of the plurality of adaptors used in the provided methods comprise a 5′ universal handle sequence, one or more unique tag sequences and a 3′ target nucleic acid sequence and a cleavable moiety. Two or more target specific adaptor pairs are included for use in provided methods, wherein each of the 3′ target specific sequences comprise cleavable moieties, each tag sequence is flanked by cleavable moieties, and each universal handle is without cleavable moieties.

In a further aspect, compositions are provided. In some embodiments provided are compositions comprising nucleic acid libraries generated by the methods described herein. In other embodiments, compositions comprising a plurality of nucleic acid adaptors are provided, wherein each of the plurality of adaptors comprise a 5′ universal handle sequence, one or more unique tag sequences, and a 3′ target nucleic acid sequence wherein each adaptor comprises a cleavable moiety. In certain embodiments the target nucleic acid sequence of the adaptor includes at least one cleavable moiety, cleavable moieties are included flanking either end of the tag sequence and the universal handle sequence does not include the cleavable moiety. In certain embodiments, compositions include at least two and up to one hundred thousand target specific adaptor pairs.

Still further, uses of provided compositions and kits comprising provided compositions for analysis of sequences of the nucleic acid libraries are additional aspects of the invention. In some embodiments, analysis of the sequences of the resulting libraries enables detection of low frequency alleles in a sample of interest.

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Efficient methods for production of targeted libraries from complex samples is desirable for a variety of nucleic acid analyses. The present invention provides, inter alia, methods of preparing libraries of target nucleic acid sequences, allowing for rapid production of highly multiplexed targeted libraries, optionally including unique tag sequences; and resulting library compositions are useful for a variety of applications, including sequencing applications. Novel features of the invention are set forth with particularity in the appended claims; and a complete understanding of the features and advantages of the present invention will be obtained by reference to the following detailed description that sets forth illustrative embodiments, in which the principles of the invention are utilized, and the accompanying drawings of which:

FIG. 1 depicts a workflow method of the invention that enables efficient rapid, highly multiplexed library preparation.

FIG. 2 depicts results from the experimental description in Example 2A.

FIG. 3 depicts results from the experimental description in Example 2B.

FIG. 4A-4C depicts results from the experimental description in Example 4.

FIG. 5 depicts results from the experimental description in Example 5.

FIG. 6A-6C depicts results from the experimental description in Example 6.

FIG. 7 depicts an additional aspect of the workflow of the invention that enables addition of adaptor sequences to facilitate bidirectional sequencing

FIG. 8 depicts an additional aspect of the workflow of the invention that enables sequencing on Illumina platforms

### DESCRIPTION OF THE INVENTION

Section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc discussed in the present teachings, such that slight and insubstantial deviations are within the scope of the present teachings herein. In this application, the use of the singular includes the plural unless specifically stated otherwise. It is noted that, as used in this specification, singular forms “a,” “an,” and “the,” and any singular use of a word, include plural referents unless expressly and unequivocally limited to one referent. Also, the use of “comprise”, “comprises”, “comprising”, “contain”, “contains”, “containing”, “include”, “includes”, and “including” are not intended to be limiting. It is to be understood that both the general description is exemplary and explanatory only and not restrictive of the invention.

Unless otherwise defined, scientific and technical terms used in connection with the invention described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization used herein are those well-known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. Techniques and procedures described herein are generally performed according to conventional methods well known in the art and as described in various general and more specific references that are cited and discussed throughout the instant specification. See. e.g., Sambrook et al., Molecular Cloning: A Laboratory Manual (Third ed., Cold Spring Harbor Laboratory Press, Cold Spring Harbor, N.Y. 2000). Unless specifically provided, any nomenclature utilized in connection with, and laboratory procedures and techniques described herein are those well-known and commonly used in the art. As utilized in accordance with embodiments provided herein, the following terms, unless otherwise indicated, shall be understood to have the following meanings:

As used herein, “amplify”, “amplifying” or “amplification reaction” and their derivatives, refer generally to an action or process whereby at least a portion of a nucleic acid molecule (referred to as a template nucleic acid molecule) is replicated or copied into at least one additional nucleic acid molecule. The additional nucleic acid molecule optionally includes sequence that is substantially identical or substantially complementary to at least some portion of the template nucleic acid molecule. A template target nucleic acid molecule may be single-stranded or double-stranded. The additional resulting replicated nucleic acid molecule may independently be single-stranded or double-stranded. In some embodiments, amplification includes a template-dependent in vitro enzyme-catalyzed reaction for the production of at least one copy of at least some portion of a target nucleic acid molecule or the production of at least one copy of a target nucleic acid sequence that is complementary to at least some portion of a target nucleic acid molecule. Amplification optionally includes linear or exponential replication of a nucleic acid molecule. In some embodiments, such amplification is performed using isothermal conditions; in other embodiments, such amplification can include thermocycling. In some embodiments, the amplification is a multiplex amplification that includes simultaneous amplification of a plurality of target sequences in a single amplification reaction. At least some target sequences can be situated on the same nucleic acid molecule or on different target nucleic acid molecules included in a single amplification reaction. In some embodiments, “amplification” includes amplification of at least some portion of DNA- and/or RNA-based nucleic acids, whether alone, or in combination. An amplification reaction can include single or double-stranded nucleic acid substrates and can further include any amplification processes known to one of ordinary skill in the art. In some embodiments, an amplification reaction includes polymerase chain reaction (PCR). In some embodiments, an amplification reaction includes isothermal amplification.

As used herein, “amplification conditions” and derivatives (e.g., conditions for amplification, etc.) generally refers to conditions suitable for amplifying one or more nucleic acid sequences. Amplification can be linear or exponential. In some embodiments, amplification conditions include isothermal conditions or alternatively include thermocyling conditions, or a combination of isothermal and thermocycling conditions. In some embodiments, conditions suitable for amplifying one or more target nucleic acid sequences includes polymerase chain reaction (PCR) conditions. Typically, amplification conditions refer to a reaction mixture that is sufficient to amplify nucleic acids such as one or more target sequences, or to amplify an amplified target sequence ligated or attached to one or more adaptors, e.g., an adaptor-attached amplified target sequence. Generally, amplification conditions include a catalyst for amplification or for nucleic acid synthesis, for example a polymerase; a primer that possesses some degree of complementarity to the nucleic acid to be amplified; and nucleotides, such as deoxyribonucleoside triphosphates (dNTPs) to promote extension of a primer once hybridized to a nucleic acid. Amplification conditions can require hybridization or annealing of a primer to a nucleic acid, extension of the primer and a denaturing step in which the extended primer is separated from the nucleic acid sequence undergoing amplification. Typically, though not necessarily, amplification conditions can include thermocycling. In some embodiments, amplification conditions include a plurality of cycles wherein steps of annealing, extending and separating are repeated. Typically, amplification conditions include cations such as Mg or Mn (e.g., MgCl₂, etc) and can also optionally include various modifiers of ionic strength.

As used herein, “target sequence” “target nucleic acid sequence” or “target sequence of interest” and derivatives, refers generally to any single or double-stranded nucleic acid sequence that can be amplified or synthesized according to the disclosure, including any nucleic acid sequence suspected or expected to be present in a sample. In some embodiments, the target sequence is present in double-stranded form and includes at least a portion of the particular nucleotide sequence to be amplified or synthesized, or its complement, prior to the addition of target-specific primers or appended adaptors. Target sequences can include the nucleic acids to which primers useful in the amplification or synthesis reaction can hybridize prior to extension by a polymerase. In some embodiments, the term refers to a nucleic acid sequence whose sequence identity, ordering or location of nucleotides is determined by one or more of the methods of the disclosure.

The term “portion” and its variants, as used herein, when used in reference to a given nucleic acid molecule, for example a primer or a template nucleic acid molecule, comprises any number of contiguous nucleotides within the length of the nucleic acid molecule, including the partial or entire length of the nucleic acid molecule.

As used herein, “contacting” and its derivatives, when used in reference to two or more components, refers generally to any process whereby the approach, proximity, mixture or commingling of the referenced components is promoted or achieved without necessarily requiring physical contact of such components, and includes mixing of solutions containing any one or more of the referenced components with each other. The referenced components may be contacted in any particular order or combination and the particular order of recitation of components is not limiting. For example, “contacting A with B and C” encompasses embodiments where A is first contacted with B then C, as well as embodiments where C is contacted with A then B, as well as embodiments where a mixture of A and C is contacted with B, and the like. Furthermore, such contacting does not necessarily require that the end result of the contacting process be a mixture including all of the referenced components, as long as at some point during the contacting process all of the referenced components are simultaneously present or simultaneously included in the same mixture or solution. For example, “contacting A with B and C” can include embodiments wherein C is first contacted with A to form a first mixture, which first mixture is then contacted with B to form a second mixture, following which C is removed from the second mixture; optionally A can then also be removed, leaving only B. Where one or more of the referenced components to be contacted includes a plurality (e.g., “contacting a target sequence with a plurality of target-specific primers and a polymerase”), then each member of the plurality can be viewed as an individual component of the contacting process, such that the contacting can include contacting of any one or more members of the plurality with any other member of the plurality and/or with any other referenced component (e.g., some but not all of the plurality of target specific primers can be contacted with a target sequence, then a polymerase, and then with other members of the plurality of target-specific primers) in any order or combination.

As used herein, the term “primer” and its derivatives refer generally to any polynucleotide that can hybridize to a target sequence of interest. In some embodiments, the primer can also serve to prime nucleic acid synthesis. Typically, a primer functions as a substrate onto which nucleotides can be polymerized by a polymerase; in some embodiments, however, a primer can become incorporated into a synthesized nucleic acid strand and provide a site to which another primer can hybridize to prime synthesis of a new strand that is complementary to the synthesized nucleic acid molecule. A primer may be comprised of any combination of nucleotides or analogs thereof, which may be optionally linked to form a linear polymer of any suitable length. In some embodiments, a primer is a single-stranded oligonucleotide or polynucleotide. (For purposes of this disclosure, the terms “polynucleotide” and “oligonucleotide” are used interchangeably herein and do not necessarily indicate any difference in length between the two). In some embodiments, a primer is double-stranded. If double stranded, a primer is first treated to separate its strands before being used to prepare extension products. Preferably, the primer is an oligodeoxyribonucleotide. A primer must be sufficiently long to prime the synthesis of extension products. Lengths of the primers will depend on many factors, including temperature, source of primer and the use of the method. In some embodiments, a primer acts as a point of initiation for amplification or synthesis when exposed to amplification or synthesis conditions; such amplification or synthesis can occur in a template-dependent fashion and optionally results in formation of a primer extension product that is complementary to at least a portion of the target sequence. Exemplary amplification or synthesis conditions can include contacting the primer with a polynucleotide template (e.g., a template including a target sequence), nucleotides and an inducing agent such as a polymerase at a suitable temperature and pH to induce polymerization of nucleotides onto an end of the target-specific primer. If double-stranded, the primer can optionally be treated to separate its strands before being used to prepare primer extension products. In some embodiments, the primer is an oligodeoxyribonucleotide or an oligoribonucleotide. In some embodiments, the primer can include one or more nucleotide analogs. The exact length and/or composition, including sequence, of the target-specific primer can influence many properties, including melting temperature (Tm), GC content, formation of secondary structures, repeat nucleotide motifs, length of predicted primer extension products, extent of coverage across a nucleic acid molecule of interest, number of primers present in a single amplification or synthesis reaction, presence of nucleotide analogs or modified nucleotides within the primers, and the like. In some embodiments, a primer can be paired with a compatible primer within an amplification or synthesis reaction to form a primer pair consisting or a forward primer and a reverse primer. In some embodiments, the forward primer of the primer pair includes a sequence that is substantially complementary to at least a portion of a strand of a nucleic acid molecule, and the reverse primer of the primer of the primer pair includes a sequence that is substantially identical to at least of portion of the strand. In some embodiments, the forward primer and the reverse primer are capable of hybridizing to opposite strands of a nucleic acid duplex. Optionally, the forward primer primes synthesis of a first nucleic acid strand, and the reverse primer primes synthesis of a second nucleic acid strand, wherein the first and second strands are substantially complementary to each other, or can hybridize to form a double-stranded nucleic acid molecule. In some embodiments, one end of an amplification or synthesis product is defined by the forward primer and the other end of the amplification or synthesis product is defined by the reverse primer. In some embodiments, where the amplification or synthesis of lengthy primer extension products is required, such as amplifying an exon, coding region, or gene, several primer pairs can be created than span the desired length to enable sufficient amplification of the region. In some embodiments, a primer can include one or more cleavable groups. In some embodiments, primer lengths are in the range of about 10 to about 60 nucleotides, about 12 to about 50 nucleotides and about 15 to about 40 nucleotides in length. Typically, a primer is capable of hybridizing to a corresponding target sequence and undergoing primer extension when exposed to amplification conditions in the presence of dNTPS and a polymerase. In some instances, the particular nucleotide sequence or a portion of the primer is known at the outset of the amplification reaction or can be determined by one or more of the methods disclosed herein. In some embodiments, the primer includes one or more cleavable groups at one or more locations within the primer.

As used herein, “target-specific primer” and its derivatives, refers generally to a single stranded or double-stranded polynucleotide, typically an oligonucleotide, that includes at least one sequence that is at least 50% complementary, typically at least 75% complementary or at least 85% complementary, more typically at least 90% complementary, more typically at least 95% complementary, more typically at least 98% or at least 99% complementary, or identical, to at least a portion of a nucleic acid molecule that includes a target sequence. In such instances, the target-specific primer and target sequence are described as “corresponding” to each other. In some embodiments, the target-specific primer is capable of hybridizing to at least a portion of its corresponding target sequence (or to a complement of the target sequence); such hybridization can optionally be performed under standard hybridization conditions or under stringent hybridization conditions. In some embodiments, the target-specific primer is not capable of hybridizing to the target sequence, or to its complement, but is capable of hybridizing to a portion of a nucleic acid strand including the target sequence, or to its complement. In some embodiments, the target-specific primer includes at least one sequence that is at least 75% complementary, typically at least 85% complementary, more typically at least 90% complementary, more typically at least 95% complementary, more typically at least 98% complementary, or more typically at least 99% complementary, to at least a portion of the target sequence itself; in other embodiments, the target-specific primer includes at least one sequence that is at least 75% complementary, typically at least 85% complementary, more typically at least 90% complementary, more typically at least 95% complementary, more typically at least 98% complementary, or more typically at least 99% complementary, to at least a portion of the nucleic acid molecule other than the target sequence. In some embodiments, the target-specific primer is substantially non-complementary to other target sequences present in the sample; optionally, the target-specific primer is substantially non-complementary to other nucleic acid molecules present in the sample. In some embodiments, nucleic acid molecules present in the sample that do not include or correspond to a target sequence (or to a complement of the target sequence) are referred to as “non-specific” sequences or “non-specific nucleic acids”. In some embodiments, the target-specific primer is designed to include a nucleotide sequence that is substantially complementary to at least a portion of its corresponding target sequence. In some embodiments, a target-specific primer is at least 95% complementary, or at least 99% complementary, or identical, across its entire length to at least a portion of a nucleic acid molecule that includes its corresponding target sequence. In some embodiments, a target-specific primer can be at least 90%, at least 95% complementary, at least 98% complementary or at least 99% complementary, or identical, across its entire length to at least a portion of its corresponding target sequence. In some embodiments, a forward target-specific primer and a reverse target-specific primer define a target-specific primer pair that can be used to amplify the target sequence via template-dependent primer extension. Typically, each primer of a target-specific primer pair includes at least one sequence that is substantially complementary to at least a portion of a nucleic acid molecule including a corresponding target sequence but that is less than 50% complementary to at least one other target sequence in the sample. In some embodiments, amplification can be performed using multiple target-specific primer pairs in a single amplification reaction, wherein each primer pair includes a forward target-specific primer and a reverse target-specific primer, each including at least one sequence that substantially complementary or substantially identical to a corresponding target sequence in the sample, and each primer pair having a different corresponding target sequence. In some embodiments, the target-specific primer can be substantially non-complementary at its 3′ end or its 5′ end to any other target-specific primer present in an amplification reaction. In some embodiments, the target-specific primer can include minimal cross hybridization to other target-specific primers in the amplification reaction. In some embodiments, target-specific primers include minimal cross-hybridization to non-specific sequences in the amplification reaction mixture. In some embodiments, the target-specific primers include minimal self-complementarity. In some embodiments, the target-specific primers can include one or more cleavable groups located at the 3′ end. In some embodiments, the target-specific primers can include one or more cleavable groups located near or about a central nucleotide of the target-specific primer. In some embodiments, one of more targets-specific primers includes only non-cleavable nucleotides at the 5′ end of the target-specific primer. In some embodiments, a target specific primer includes minimal nucleotide sequence overlap at the 3′ end or the 5′ end of the primer as compared to one or more different target-specific primers, optionally in the same amplification reaction. In some embodiments 1, 2, 3, 4, 5, 6, 7, 8, 9, 10 or more, target-specific primers in a single reaction mixture include one or more of the above embodiments. In some embodiments, substantially all of the plurality of target-specific primers in a single reaction mixture includes one or more of the above embodiments.

As used herein, the term “adaptor” denotes a nucleic acid molecule that can be used for manipulation of a polynucleotide of interest. In some embodiments, adaptors are used for amplification of one or more target nucleic acids. In some embodiments, the adaptors are used in reactions for sequencing. In some embodiments, an adaptor has one or more ends that lack a 5′ phosphate residue. In some embodiments, an adaptor comprises, consists of, or consist essentially of at least one priming site. Such priming site containing adaptors can be referred to as “primer” adaptors. In some embodiments, the adaptor priming site can be useful in PCR processes. In some embodiments an adaptor includes a nucleic acid sequence that is substantially complementary to the 3′ end or the 5′ end of at least one target sequences within the sample, referred to herein as a gene specific target sequence, a target specific sequence, or target specific primer. In some embodiments, the adaptor includes nucleic acid sequence that is substantially non-complementary to the 3′ end or the 5′ end of any target sequence present in the sample. In some embodiments, the adaptor includes single stranded or double-stranded linear oligonucleotide that is not substantially complementary to an target nucleic acid sequence. In some embodiments, the adaptor includes nucleic acid sequence that is substantially non-complementary to at least one, and preferably some or all of the nucleic acid molecules of the sample. In some embodiments, suitable adaptor lengths are in the range of about 10-75 nucleotides, about 12-50 nucleotides and about 15-40 nucleotides in length. Generally, an adaptor can include any combination of nucleotides and/or nucleic acids. In some aspects, adaptors include one or more cleavable groups at one or more locations. In some embodiments, the adaptor includes sequence that is substantially identical, or substantially complementary, to at least a portion of a primer, for example a universal primer. In some embodiments, adaptors include a tag sequence to assist with cataloguing, identification or sequencing. In some embodiments, an adaptor acts as a substrate for amplification of a target sequence, particularly in the presence of a polymerase and dNTPs under suitable temperature and pH.

As used herein, “polymerase” and its derivatives, generally refers to any enzyme that can catalyze the polymerization of nucleotides (including analogs thereof) into a nucleic acid strand. Typically but not necessarily, such nucleotide polymerization can occur in a template-dependent fashion. Such polymerases can include without limitation naturally occurring polymerases and any subunits and truncations thereof, mutant polymerases, variant polymerases, recombinant, fusion or otherwise engineered polymerases, chemically modified polymerases, synthetic molecules or assemblies, and any analogs, derivatives or fragments thereof that retain the ability to catalyze such polymerization. Optionally, the polymerase can be a mutant polymerase comprising one or more mutations involving the replacement of one or more amino acids with other amino acids, the insertion or deletion of one or more amino acids from the polymerase, or the linkage of parts of two or more polymerases. Typically, the polymerase comprises one or more active sites at which nucleotide binding and/or catalysis of nucleotide polymerization can occur. Some exemplary polymerases include without limitation DNA polymerases and RNA polymerases. The term “polymerase” and its variants, as used herein, also refers to fusion proteins comprising at least two portions linked to each other, where the first portion comprises a peptide that can catalyze the polymerization of nucleotides into a nucleic acid strand and is linked to a second portion that comprises a second polypeptide. In some embodiments, the second polypeptide can include a reporter enzyme or a processivity-enhancing domain. Optionally, the polymerase can possess 5′ exonuclease activity or terminal transferase activity. In some embodiments, the polymerase can be optionally reactivated, for example through the use of heat, chemicals or re-addition of new amounts of polymerase into a reaction mixture. In some embodiments, the polymerase can include a hot-start polymerase and/or an aptamer based polymerase that optionally can be reactivated.

The terms “identity” and “identical” and their variants, as used herein, when used in reference to two or more nucleic acid sequences, refer to similarity in sequence of the two or more sequences (e.g., nucleotide or polypeptide sequences). In the context of two or more homologous sequences, the percent identity or homology of the sequences or subsequences thereof indicates the percentage of all monomeric units (e.g., nucleotides or amino acids) that are the same (i.e., about 70% identity, preferably 75%, 80%, 85%, 90%, 95%, 98% or 99% identity). The percent identity can be over a specified region, when compared and aligned for maximum correspondence over a comparison window, or designated region as measured using a BLAST or BLAST 2.0 sequence comparison algorithms with default parameters described below, or by manual alignment and visual inspection. Sequences are said to be “substantially identical” when there is at least 85% identity at the amino acid level or at the nucleotide level. Preferably, the identity exists over a region that is at least about 25, 50, or 100 residues in length, or across the entire length of at least one compared sequence. A typical algorithm for determining percent sequence identity and sequence similarity are the BLAST and BLAST 2.0 algorithms, which are described in Altschul et al, Nuc. Acids Res. 25:3389-3402 (1977). Other methods include the algorithms of Smith & Waterman, Adv. Appl. Math. 2:482 (1981), and Needleman & Wunsch, J. Mol. Biol. 48:443 (1970), etc. Another indication that two nucleic acid sequences are substantially identical is that the two molecules or their complements hybridize to each other under stringent hybridization conditions.

The terms “complementary” and “complement” and their variants, as used herein, refer to any two or more nucleic acid sequences (e.g., portions or entireties of template nucleic acid molecules, target sequences and/or primers) that can undergo cumulative base pairing at two or more individual corresponding positions in antiparallel orientation, as in a hybridized duplex. Such base pairing can proceed according to any set of established rules, for example according to Watson-Crick base pairing rules or according to some other base pairing paradigm. Optionally there can be “complete” or “total” complementarity between a first and second nucleic acid sequence where each nucleotide in the first nucleic acid sequence can undergo a stabilizing base pairing interaction with a nucleotide in the corresponding antiparallel position on the second nucleic acid sequence. “Partial” complementarity describes nucleic acid sequences in which at least 20%, but less than 100%, of the residues of one nucleic acid sequence are complementary to residues in the other nucleic acid sequence. In some embodiments, at least 50%, but less than 100%, of the residues of one nucleic acid sequence are complementary to residues in the other nucleic acid sequence. In some embodiments, at least 70%, 80%, 90%, 95% or 98%, but less than 100%, of the residues of one nucleic acid sequence are complementary to residues in the other nucleic acid sequence. Sequences are said to be “substantially complementary” when at least 85% of the residues of one nucleic acid sequence are complementary to residues in the other nucleic acid sequence. In some embodiments, two complementary or substantially complementary sequences are capable of hybridizing to each other under standard or stringent hybridization conditions. “Non-complementary” describes nucleic acid sequences in which less than 20% of the residues of one nucleic acid sequence are complementary to residues in the other nucleic acid sequence. Sequences are said to be “substantially non-complementary” when less than 15% of the residues of one nucleic acid sequence are complementary to residues in the other nucleic acid sequence. In some embodiments, two non-complementary or substantially non-complementary sequences cannot hybridize to each other under standard or stringent hybridization conditions. A “mismatch” is present at any position in the two opposed nucleotides are not complementary. Complementary nucleotides include nucleotides that are efficiently incorporated by DNA polymerases opposite each other during DNA replication under physiological conditions. In a typical embodiment, complementary nucleotides can form base pairs with each other, such as the A-T/U and G-C base pairs formed through specific Watson-Crick type hydrogen bonding, or base pairs formed through some other type of base pairing paradigm, between the nucleobases of nucleotides and/or polynucleotides in positions antiparallel to each other. The complementarity of other artificial base pairs can be based on other types of hydrogen bonding and/or hydrophobicity of bases and/or shape complementarity between bases.

As used herein, “amplified target sequences” and its derivatives, refers generally to a nucleic acid sequence produced by the amplification of/amplifying the target sequences using target-specific primers and the methods provided herein. The amplified target sequences may be either of the same sense (the positive strand produced in the second round and subsequent even-numbered rounds of amplification) or antisense (i.e., the negative strand produced during the first and subsequent odd-numbered rounds of amplification) with respect to the target sequences. For the purposes of this disclosure, amplified target sequences are typically less than 50% complementary to any portion of another amplified target sequence in the reaction.

As used herein, terms “ligating”, “ligation” and derivatives refer generally to the act or process for covalently linking two or more molecules together, for example, covalently linking two or more nucleic acid molecules to each other. In some embodiments, ligation includes joining nicks between adjacent nucleotides of nucleic acids. In some embodiments, ligation includes forming a covalent bond between an end of a first and an end of a second nucleic acid molecule. In some embodiments, for example embodiments wherein the nucleic acid molecules to be ligated include conventional nucleotide residues, the ligation can include forming a covalent bond between a 5′ phosphate group of one nucleic acid and a 3′ hydroxyl group of a second nucleic acid thereby forming a ligated nucleic acid molecule. In some embodiments, any means for joining nicks or bonding a 5′ phosphate to a 3′ hydroxyl between adjacent nucleotides can be employed. In an exemplary embodiment, an enzyme such as a ligase can be used.

As used herein, “ligase” and its derivatives, refers generally to any agent capable of catalyzing the ligation of two substrate molecules. In some embodiments, the ligase includes an enzyme capable of catalyzing the joining of nicks between adjacent nucleotides of a nucleic acid. In some embodiments, a ligase includes an enzyme capable of catalyzing the formation of a covalent bond between a 5′ phosphate of one nucleic acid molecule to a 3′ hydroxyl of another nucleic acid molecule thereby forming a ligated nucleic acid molecule. Suitable ligases may include, but not limited to, T4 DNA ligase; T7 DNA ligase; Taq DNA ligase, and E. coli DNA ligase.

As defined herein, a “cleavable group” generally refers to any moiety that once incorporated into a nucleic acid can be cleaved under appropriate conditions. For example, a cleavable group can be incorporated into a target-specific primer, an amplified sequence, an adaptor or a nucleic acid molecule of the sample. In an exemplary embodiment, a target-specific primer can include a cleavable group that becomes incorporated into the amplified product and is subsequently cleaved after amplification, thereby removing a portion, or all, of the target-specific primer from the amplified product. The cleavable group can be cleaved or otherwise removed from a target-specific primer, an amplified sequence, an adaptor or a nucleic acid molecule of the sample by any acceptable means. For example, a cleavable group can be removed from a target-specific primer, an amplified sequence, an adaptor or a nucleic acid molecule of the sample by enzymatic, thermal, photo-oxidative or chemical treatment. In one aspect, a cleavable group can include a nucleobase that is not naturally occurring. For example, an oligodeoxyribonucleotide can include one or more RNA nucleobases, such as uracil that can be removed by a uracil glycosylase. In some embodiments, a cleavable group can include one or more modified nucleobases (such as 7-methylguanine, 8-oxo-guanine, xanthine, hypoxanthine, 5,6-dihydrouracil or 5-methylcytosine) or one or more modified nucleosides (i.e., 7-methylguanosine, 8-oxo-deoxyguanosine, xanthosine, inosine, dihydrouridine or 5-methylcytidine). The modified nucleobases or nucleotides can be removed from the nucleic acid by enzymatic, chemical or thermal means. In one embodiment, a cleavable group can include a moiety that can be removed from a primer after amplification (or synthesis) upon exposure to ultraviolet light (i.e., bromodeoxyuridine). In another embodiment, a cleavable group can include methylated cytosine. Typically, methylated cytosine can be cleaved from a primer for example, after induction of amplification (or synthesis), upon sodium bisulfite treatment. In some embodiments, a cleavable moiety can include a restriction site. For example, a primer or target sequence can include a nucleic acid sequence that is specific to one or more restriction enzymes, and following amplification (or synthesis), the primer or target sequence can be treated with the one or more restriction enzymes such that the cleavable group is removed. Typically, one or more cleavable groups can be included at one or more locations with a target-specific primer, an amplified sequence, an adaptor or a nucleic acid molecule of the sample.

As used herein, “digestion”, “digestion step” and its derivatives, generally refers to any process by which a cleavable group is cleaved or otherwise removed from a target-specific primer, an amplified sequence, an adaptor or a nucleic acid molecule of the sample. In some embodiments, the digestion step involves a chemical, thermal, photo-oxidative or digestive process.

As used herein, the term “hybridization” is consistent with its use in the art, and generally refers to the process whereby two nucleic acid molecules undergo base pairing interactions. Two nucleic acid molecule molecules are said to be hybridized when any portion of one nucleic acid molecule is base paired with any portion of the other nucleic acid molecule; it is not necessarily required that the two nucleic acid molecules be hybridized across their entire respective lengths and in some embodiments, at least one of the nucleic acid molecules can include portions that are not hybridized to the other nucleic acid molecule. The phrase “hybridizing under stringent conditions” and its variants refers generally to conditions under which hybridization of a target-specific primer to a target sequence occurs in the presence of high hybridization temperature and low ionic strength. As used herein, the phrase “standard hybridization conditions” and its variants refers generally to conditions under which hybridization of a primer to an oligonucleotide (i.e., a target sequence), occurs in the presence of low hybridization temperature and high ionic strength. In one exemplary embodiment, standard hybridization conditions include an aqueous environment containing about 100 mm magnesium sulfate, about 500 mM Tris-sulfate at pH 8.9, and about 200 mM ammonium sulfate at about 50-55° C., or equivalents thereof.

As used herein, the term “end” and its variants, when used in reference to a nucleic acid molecule, for example a target sequence or amplified target sequence, can include the terminal 30 nucleotides, the terminal 20 and even more typically the terminal 15 nucleotides of the nucleic acid molecule. A linear nucleic acid molecule comprised of linked series of contiguous nucleotides typically includes at least two ends. In some embodiments, one end of the nucleic acid molecule can include a 3′ hydroxyl group or its equivalent, and can be referred to as the “3′ end” and its derivatives. Optionally, the 3′ end includes a 3′ hydroxyl group that is not linked to a 5′ phosphate group of a mononucleotide pentose ring. Typically, the 3′ end includes one or more 5′ linked nucleotides located adjacent to the nucleotide including the unlinked 3′ hydroxyl group, typically the 30 nucleotides located adjacent to the 3′ hydroxyl, typically the terminal 20 and even more typically the terminal 15 nucleotides. Generally, the one or more linked nucleotides can be represented as a percentage of the nucleotides present in the oligonucleotide or can be provided as a number of linked nucleotides adjacent to the unlinked 3′ hydroxyl. For example, the 3′ end can include less than 50% of the nucleotide length of the oligonucleotide. In some embodiments, the 3′ end does not include any unlinked 3′ hydroxyl group but can include any moiety capable of serving as a site for attachment of nucleotides via primer extension and/or nucleotide polymerization. In some embodiments, the term “3′ end” for example when referring to a target-specific primer, can include the terminal 10 nucleotides, the terminal 5 nucleotides, the terminal 4, 3, 2 or fewer nucleotides at the 3′ end. In some embodiments, the term “3′ end” when referring to a target-specific primer can include nucleotides located at nucleotide positions 10 or fewer from the 3′ terminus. As used herein, “5′ end”, and its derivatives, generally refers to an end of a nucleic acid molecule, for example a target sequence or amplified target sequence, which includes a free 5′ phosphate group or its equivalent. In some embodiments, the 5′ end includes a 5′ phosphate group that is not linked to a 3′ hydroxyl of a neighboring mononucleotide pentose ring. Typically, the 5′ end includes to one or more linked nucleotides located adjacent to the 5′ phosphate, typically the 30 nucleotides located adjacent to the nucleotide including the 5′ phosphate group, typically the terminal 20 and even more typically the terminal 15 nucleotides. Generally, the one or more linked nucleotides can be represented as a percentage of the nucleotides present in the oligonucleotide or can be provided as a number of linked nucleotides adjacent to the 5′ phosphate. For example, the 5′ end can be less than 50% of the nucleotide length of an oligonucleotide. In another exemplary embodiment, the 5′ end can include about 15 nucleotides adjacent to the nucleotide including the terminal 5′ phosphate. In some embodiments, the 5′ end does not include any unlinked 5′ phosphate group but can include any moiety capable of serving as a site of attachment to a 3′ hydroxyl group, or to the 3′ end of another nucleic acid molecule. In some embodiments, the term “5′ end” for example when referring to a target-specific primer, can include the terminal 10 nucleotides, the terminal 5 nucleotides, the terminal 4, 3, 2 or fewer nucleotides at the 5′ end. In some embodiments, the term “5′ end” when referring to a target-specific primer can include nucleotides located at positions 10 or fewer from the 5′ terminus. In some embodiments, the 5′ end of a target-specific primer can include only non-cleavable nucleotides, for example nucleotides that do not contain one or more cleavable groups as disclosed herein, or a cleavable nucleotide as would be readily determined by one of ordinary skill in the art. A “first end” and a “second end” of a polynucleotide refer to the 5′ end or the 3′ end of the polynucleotide. Either the first end or second end of a polynucleotide can be the 5′ end or the 3′ end of the polynucleotide; the terms “first” and “second” are not meant to denote that the end is specifically the 5′ end or the 3′ end.

As used herein “tag,” “barcode,” “unique tag” or “tag sequence” and its derivatives, refers generally to a unique short (6-14 nucleotide) nucleic acid sequence within an adaptor or primer that can act as a ‘key’ to distinguish or separate a plurality of amplified target sequences in a sample. For the purposes of this disclosure, a barcode or unique tag sequence is incorporated into the nucleotide sequence of an adaptor or primer. As used herein, “barcode sequence” denotes a nucleic acid fixed sequence that is sufficient to allow for the identification of a sample or source of nucleic acid sequences of interest. A barcode sequence can be, but need not be, a small section of the original nucleic acid sequence on which the identification is to be based. In some embodiments a barcode is 5-20 nucleic acids long. In some embodiments, the barcode is comprised of analog nucleotides, such as L-DNA, LNA, PNA, etc. As used herein, “unique tag sequence” denotes a nucleic acid sequence having at least one random sequence and at least one fixed sequence. A unique tag sequence, alone or in conjunction with a second unique tag sequence, is sufficient to allow for the identification of a single target nucleic acid molecule in a sample. A unique tag sequence can, but need not, comprise a small section of the original target nucleic acid sequence. In some embodiments a unique tag sequence is 2-50 nucleotides or base-pairs, or 2-25 nucleotides or base-pairs, or 2-10 nucleotides or base-pairs in length. A unique tag sequence can comprise at least one random sequence interspersed with a fixed sequence.

As used herein, “comparable maximal minimum melting temperatures” and its derivatives, refers generally to the melting temperature (Tm) of each nucleic acid fragment for a single adaptor or target-specific primer after digestion of a cleavable groups. The hybridization temperature of each nucleic acid fragment generated by an adaptor or target-specific primer is compared to determine the maximal minimum temperature required preventing hybridization of a nucleic acid sequence from the target-specific primer or adaptor or fragment or portion thereof to a respective target sequence. Once the maximal hybridization temperature is known, it is possible to manipulate the adaptor or target-specific primer, for example by moving the location of one or more cleavable group(s) along the length of the primer, to achieve a comparable maximal minimum melting temperature with respect to each nucleic acid fragment to thereby optimize digestion and repair steps of library preparation.

As used herein, “addition only” and its derivatives, refers generally to a series of steps in which reagents and components are added to a first or single reaction mixture. Typically, the series of steps excludes the removal of the reaction mixture from a first vessel to a second vessel in order to complete the series of steps. Generally, an addition only process excludes the manipulation of the reaction mixture outside the vessel containing the reaction mixture. Typically, an addition-only process is amenable to automation and high-throughput.

As used herein, “polymerizing conditions” and its derivatives, refers generally to conditions suitable for nucleotide polymerization. In typical embodiments, such nucleotide polymerization is catalyzed by a polymerase. In some embodiments, polymerizing conditions include conditions for primer extension, optionally in a template-dependent manner, resulting in the generation of a synthesized nucleic acid sequence. In some embodiments, the polymerizing conditions include polymerase chain reaction (PCR). Typically, the polymerizing conditions include use of a reaction mixture that is sufficient to synthesize nucleic acids and includes a polymerase and nucleotides. The polymerizing conditions can include conditions for annealing of a target-specific primer to a target sequence and extension of the primer in a template dependent manner in the presence of a polymerase. In some embodiments, polymerizing conditions can be practiced using thermocycling. Additionally, polymerizing conditions can include a plurality of cycles where the steps of annealing, extending, and separating the two nucleic strands are repeated. Typically, the polymerizing conditions include a cation such as MgCl₂. Generally, polymerization of one or more nucleotides to form a nucleic acid strand includes that the nucleotides be linked to each other via phosphodiester bonds, however, alternative linkages may be possible in the context of particular nucleotide analogs.

As used herein, the term “nucleic acid” refers to natural nucleic acids, artificial nucleic acids, analogs thereof, or combinations thereof, including polynucleotides and oligonucleotides. As used herein, the terms “polynucleotide” and “oligonucleotide” are used interchangeably and mean single-stranded and double-stranded polymers of nucleotides including, but not limited to, 2′-deoxyribonucleotides (nucleic acid) and ribonucleotides (RNA) linked by internucleotide phosphodiester bond linkages, e.g. 3′-5′ and 2′-5′, inverted linkages, e.g. 3′-3′ and 5′-5′, branched structures, or analog nucleic acids. Polynucleotides have associated counter ions, such as H⁺, NH₄⁺, trialkylammonium, Mg²⁺, Na⁺ and the like. An oligonucleotide can be composed entirely of deoxyribonucleotides, entirely of ribonucleotides, or chimeric mixtures thereof. Oligonucleotides can be comprised of nucleobase and sugar analogs. Polynucleotides typically range in size from a few monomeric units, e.g. 5-40, when they are more commonly frequently referred to in the art as oligonucleotides, to several thousands of monomeric nucleotide units, when they are more commonly referred to in the art as polynucleotides; for purposes of this disclosure, however, both oligonucleotides and polynucleotides may be of any suitable length. Unless denoted otherwise, whenever a oligonucleotide sequence is represented, it will be understood that the nucleotides are in 5′ to 3′ order from left to right and that “A” denotes deoxyadenosine, “C” denotes deoxycytidine, “G” denotes deoxyguanosine, “T” denotes thymidine, and “U’ denotes deoxyuridine. As discussed herein and known in the art, oligonucleotides and polynucleotides are said to have “5′ ends” and “3′ ends” because mononucleotides are typically reacted to form oligonucleotides via attachment of the 5′ phosphate or equivalent group of one nucleotide to the 3′ hydroxyl or equivalent group of its neighboring nucleotide, optionally via a phosphodiester or other suitable linkage.

As used herein, the term “polymerase chain reaction” (“PCR”) refers to the method of K. B. Mullis U.S. Pat. Nos. 4,683,195 and 4,683,202, hereby incorporated by reference, which describe a method for increasing the concentration of a segment of a polynucleotide of interest in a mixture of genomic DNA without cloning or purification. This process for amplifying the polynucleotide of interest consists of introducing a large excess of two oligonucleotide primers to the DNA mixture containing the desired polynucleotide of interest, followed by a precise sequence of thermal cycling in the presence of a DNA polymerase. The two primers are complementary to their respective strands of the double stranded polynucleotide of interest. To effect amplification, the mixture is denatured and the primers then annealed to their complementary sequences within the polynucleotide of interest molecule. Following annealing, the primers are extended with a polymerase to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired polynucleotide of interest. The length of the amplified segment of the desired polynucleotide of interest (amplicon) is determined by the relative positions of the primers with respect to each other, and therefore, this length is a controllable parameter. By virtue of repeating the process, the method is referred to as the “polymerase chain reaction” (hereinafter “PCR”). Because the desired amplified segments of the polynucleotide of interest become the predominant nucleic acid sequences (in terms of concentration) in the mixture, they are said to be “PCR amplified”. As defined herein, target nucleic acid molecules within a sample including a plurality of target nucleic acid molecules are amplified via PCR. In a modification to the method discussed above, the target nucleic acid molecules can be PCR amplified using a plurality of different primer pairs, in some cases, one or more primer pairs per target nucleic acid molecule of interest, thereby forming a multiplex PCR reaction. Using multiplex PCR, it is possible to simultaneously amplify multiple nucleic acid molecules of interest from a sample to form amplified target sequences. It is also possible to detect the amplified target sequences by several different methodologies (e.g., quantitation with a bioanalyzer or qPCR, hybridization with a labeled probe; incorporation of biotinylated primers followed by avidin-enzyme conjugate detection; incorporation of ³²P-labeled deoxynucleotide triphosphates, such as dCTP or dATP, into the amplified target sequence). Any oligonucleotide sequence can be amplified with the appropriate set of primers, thereby allowing for the amplification of target nucleic acid molecules from genomic DNA, cDNA, formalin-fixed paraffin-embedded DNA, fine-needle biopsies and various other sources. In particular, the amplified target sequences created by the multiplex PCR process as disclosed herein, are themselves efficient substrates for subsequent PCR amplification or various downstream assays or manipulations.

As defined herein “multiplex amplification” refers to selective and non-random amplification of two or more target sequences within a sample using at least one target-specific primer. In some embodiments, multiplex amplification is performed such that some or all of the target sequences are amplified within a single reaction vessel. The “plexy” or “plex” of a given multiplex amplification refers generally to the number of different target-specific sequences that are amplified during that single multiplex amplification. In some embodiments, the plexy can be about 12-plex, 24-plex, 48-plex, 96-plex, 192-plex, 384-plex, 768-plex, 1536-plex, 3072-plex, 6144-plex or higher.

### Methods of Preparing Nucleic Acid Libraries

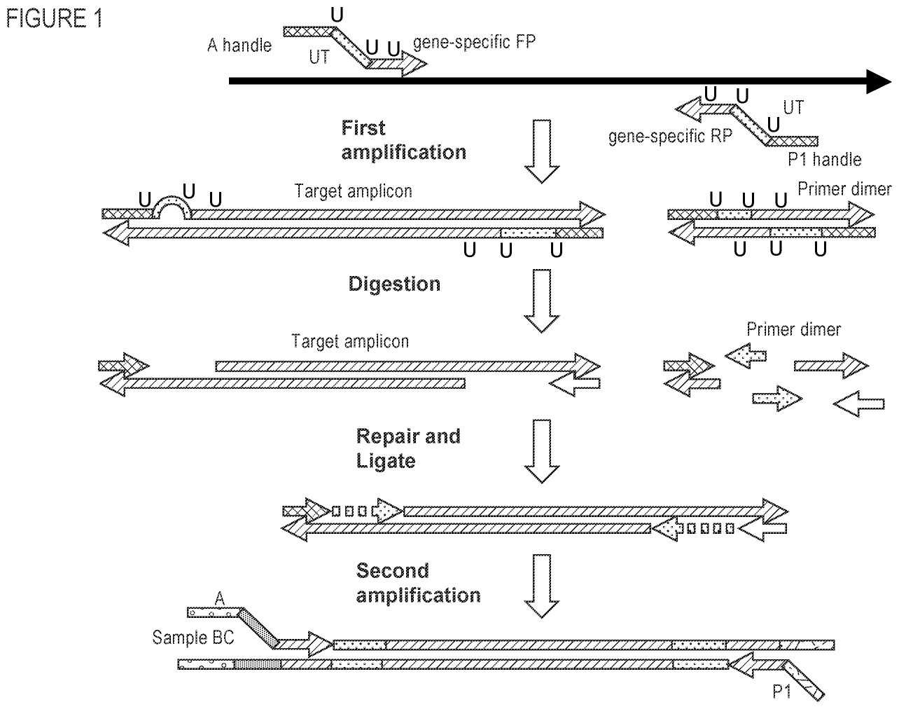

Provided methods of the invention comprise efficient procedures which enable rapid preparation of highly multiplexed libraries suitable for downstream analysis. See FIG. 1. The methods optionally allow for incorporation of one or more unique tag sequences, if so desired. Certain methods comprise streamlined, addition-only procedures conveying highly rapid library generation.

In one aspect of the invention, methods for preparing a library of target nucleic acid sequences are provided. In some embodiments, methods comprise contacting a nucleic acid sample with a plurality of adaptors capable of amplification of one or more target nucleic acid sequences in the sample under conditions wherein the target nucleic acid(s) undergo a first amplification; digesting resulting first amplification products to reduce or eliminate resulting primer dimers and prepare partially digested target amplicons, thereby producing gapped, double stranded amplicons. The methods further comprise repairing the partially digested target amplicons; then amplifying the repaired target amplicons in a second amplification using universal primers, thereby producing a library of target nucleic acid sequences. Each of the plurality of adaptors used in the methods herein comprise a universal handle sequence and a target nucleic acid sequence and a cleavable moiety and optionally one or more tag sequences. At least two and up to one hundred thousand target specific adaptor pairs are included in the provided methods, wherein the target nucleic acid sequence of each adaptor includes at least one cleavable moiety and the universal handle sequence does not include the cleavable moiety. In some embodiments where an optional tag sequence is included in at least one adaptor, the cleavable moieties are included in the adaptor sequence flanking either end of the tag sequence.

In one aspect of the invention, methods for preparing a tagged library of target nucleic acid sequences are provided. In some embodiments, methods comprise contacting a nucleic acid sample with a plurality of adaptors capable of amplification of one or more target nucleic acid sequences in the sample under conditions wherein the target nucleic acid(s) undergo a first amplification; digesting resulting first amplification products to reduce or eliminate resulting primer dimers and prepare partially digested target amplicons, thereby producing gapped, double stranded amplicons. The methods further comprise repairing the partially digested target amplicons; then amplifying the repaired target amplicons in a second amplification using universal primers, thereby producing a library of target nucleic acid sequences. Each of the plurality of adaptors used in the methods herein comprise a universal handle sequence and a target nucleic acid sequence and a cleavable moiety and one or more tag sequences. At least two and up to one hundred thousand target specific adaptor pairs are included in the provided methods, wherein the target nucleic acid sequence of each adaptor includes at least one cleavable moiety, the universal handle sequence does not include the cleavable moiety, and the cleavable moieties are included flanking either end of the tag sequence.

In certain embodiments, the comparable maximal minimum melting temperature of each universal sequence is higher than the comparable maximal minimum melting temperature of each target nucleic acid sequence and each tag sequence present in an adaptor.

In some embodiments, each of the adaptors comprise unique tag sequences as further described herein and each further comprise cleavable groups flanking either end of the tag sequence in each adaptor. In some embodiments wherein unique taq sequences are employed, each generated target specific amplicon sequence includes at least 1 different sequence and up to 107 different sequences. In certain embodiments each target specific pair of the plurality of adaptors includes up to 16,777,216 different adaptor combinations comprising different tag sequences.

In some embodiments, methods comprise contacting the plurality of gapped polynucleotide products with digestion and repair reagents simultaneously. In some embodiments, methods comprise contacting the plurality of gapped polynucleotide products sequentially with the digestion then repair reagents.

A digestion reagent useful in the methods provided herein comprises any reagent capable of cleaving the cleavable site present in adaptors, and in some embodiments includes, but is not limited to, one or a combination of uracil DNA glycosylase (UDG). apurinic endonuclease (e.g., APE1), RecJf, formamidopyrimidine [fapy]-DNA glycosylase (fpg), Nth endonuclease III, endonuclease VIII, polynucleotide kinase (PNK), Taq DNA polymerase, DNA polymerase I and/or human DNA polymerase beta.

A repair reagent useful in the methods provided herein comprises any reagent capable of repair of the gapped amplicons, and in some embodiments includes, but is not limited to, any one or a combination of Phusion DNA polymerase, Phusion U DNA polymerase, SuperFi DNA polymerase, Taq DNA polymerase, Human DNA polymerase beta, T4 DNA polymerase and/or T7 DNA polymerase, SuperFiU DNA polymerase, E. coli DNA ligase, T3 DNA ligase, T4 DNA ligase, T7 DNA ligase, Taq DNA ligase, and/or 9° N DNA ligase.

Thus, in certain embodiments, a digestion and repair reagent comprises any one or a combination of one or a combination of uracil DNA glycosylase (UDG). apurinic endonuclease (e.g., APE1), RecJf, formamidopyrimidine [fapy]-DNA glycosylase (fpg), Nth endonuclease III, endonuclease VIII, polynucleotide kinase (PNK), Taq DNA polymerase, DNA polymerase I and/or human DNA polymerase beta; and any one or a combination of Phusion DNA polymerase, Phusion U DNA polymerase, SuperFi DNA polymerase, Taq DNA polymerase, Human DNA polymerase beta, T4 DNA polymerase and/or T7 DNA polymerase, SuperFiU DNA polymerase, E. coli DNA ligase, T3 DNA ligase, T4 DNA ligase, T7 DNA ligase, Taq DNA ligase, and/or 9° N DNA ligase. In certain embodiments, a digestion and repair reagent comprises any one or a combination of uracil DNA glycosylase (UDG), apurinic endonuclease (e.g., APE1), Taq DNA polymerase, Phusion U DNA polymerase, SuperFiU DNA polymerase, T7 DNA ligase. In certain embodiments, a digestion and repair reagent comprises any one or a combination of uracil DNA glycosylase (UDG), formamidopyrimidine [fapy]-DNA glycosylase (fpg), Phusion U DNA polymerase, Taq DNA polymerase, SuperFiU DNA polymerase, T4 PNK and T7 DNA ligase.

In some embodiments, methods comprise the digestion and repair steps carried out in a single step. In other embodiments, methods comprise the digestion and repair of steps carried out in a temporally separate manner at different temperatures.

In some embodiments methods of the invention are carried out wherein one or more of the method steps is conducted in manual mode. In particular embodiments, methods of the invention are carried out wherein each of the method steps is conducted manually. In some embodiments methods of the invention are carried out wherein one or more of the method steps is conducted in an automated mode. In particular embodiments, methods of the invention are carried wherein each of the method steps is automated. In some embodiments methods of the invention are carried out wherein one or more of the method steps is conducted in a combination of manual and automated modes.

In some embodiments, methods of the invention comprise at least one purification step. For example, in certain embodiments a purification step is carried out only after the second amplification of repaired amplicons. In some embodiments two purification steps are utilized, wherein a first purification step is carried out after the digestion and repair and a second purification step is carried out after the second amplification of repaired amplicons.

In some embodiments a purification step comprises conducting a solid phase adherence reaction, solid phase immobilization reaction or gel electrophoresis. In certain embodiments a purification step comprises separation conducted using Solid Phase Reversible Immobilization (SPRI) beads. In particular embodiments a purification step comprises separation conducted using SPRI beads wherein the SPRI beads comprise paramagnetic beads.

In some embodiments, methods comprise contacting a nucleic acid sample with a plurality of adaptors capable of amplification of one or more target nucleic acid sequences in the sample under conditions wherein the target nucleic acid(s) undergo a first amplification; digesting resulting first amplification products to reduce or eliminate resulting primer dimers and prepare partially digested target amplicons, thereby producing gapped, double stranded amplicons. The methods further comprise repairing the partially digested target amplicons, then purifying repaired amplicons; then amplifying the repaired target amplicons in a second amplification using universal primers, thereby producing a library of target nucleic acid sequences; and then purifying resulting library. Each of the plurality of adaptors used in the methods herein comprise a universal handle sequence and a target nucleic acid sequence and a cleavable moiety and optionally one or more tag sequences. At least two and up to one hundred thousand target specific adaptor pairs are included in the provided methods, wherein the target nucleic acid sequence of each adaptor includes at least one cleavable moiety and the universal handle sequence does not include the cleavable moiety. In some embodiments where an optional tag sequence is included in at least one adaptor, the cleavable moieties are included in the adaptor sequence flanking either end of the tag sequence.

In some embodiments, methods comprise contacting a nucleic acid sample with a plurality of adaptors capable of amplification of one or more target nucleic acid sequences in the sample under conditions wherein the target nucleic acid(s) undergo a first amplification; digesting resulting first amplification products to reduce or eliminate resulting primer dimers and prepare partially digested target amplicons, thereby producing gapped, double stranded amplicons. The methods further comprise repairing the partially digested target amplicons, and purifying repaired amplicons; then amplifying the repaired target amplicons in a second amplification using universal primers, thereby producing a library of target nucleic acid sequences; and then purifying resulting library. Each of the plurality of adaptors used in the methods herein comprise a universal handle sequence and a target nucleic acid sequence and a cleavable moiety and one or more tag sequences. At least two and up to one hundred thousand target specific adaptor pairs are included in the provided methods, wherein the target nucleic acid sequence of each adaptor includes at least one cleavable moiety, the universal handle sequence does not include the cleavable moiety, and cleavable moieties are included in the flanking either end of the tag sequence.

In some embodiments, methods comprise contacting a nucleic acid sample with a plurality of adaptors capable of amplification of one or more target nucleic acid sequences in the sample under conditions wherein the target nucleic acid(s) undergo a first amplification; digesting resulting first amplification products to reduce or eliminate resulting primer dimers and prepare partially digested target amplicons, thereby producing gapped, double stranded amplicons. The methods further comprise repairing the partially digested target amplicons, then purifying repaired amplicons; then amplifying the repaired target amplicons in a second amplification using universal primers, thereby producing a library of target nucleic acid sequences; and then purifying resulting library. Each of the plurality of adaptors used in the methods herein comprise a universal handle sequence and a target nucleic acid sequence and a cleavable moiety and optionally one or more tag sequences. At least two and up to one hundred thousand target specific adaptor pairs are included in the provided methods, wherein the target nucleic acid sequence of each adaptor includes at least one cleavable moiety and the universal handle sequence does not include the cleavable moiety. In some embodiments where an optional tag sequence is included in at least one adaptor, the cleavable moieties are included in the adaptor sequence flanking either end of the tag sequence. In some embodiments a digestion and repair reagent comprises any one or a combination of one or a combination of uracil DNA glycosylase (UDG). apurinic endonuclease (e.g., APE1), RecJf, formamidopyrimidine [fapy]-DNA glycosylase (fpg), Nth endonuclease III, endonuclease VIII, polynucleotide kinase (PNK), Taq DNA polymerase, DNA polymerase I and/or human DNA polymerase beta; and any one or a combination of Phusion DNA polymerase, Phusion U DNA polymerase, SuperFi DNA polymerase, Taq DNA polymerase, Human DNA polymerase beta, T4 DNA polymerase and/or T7 DNA polymerase, SuperFiU DNA polymerase, E. coli DNA ligase, T3 DNA ligase, T4 DNA ligase, T7 DNA ligase, Taq DNA ligase, and/or 9° N DNA ligase. In certain embodiments, a digestion and repair reagent comprises any one or a combination of uracil DNA glycosylase (UDG), apurinic endonuclease (e.g., APE1), Taq DNA polymerase, Phusion U DNA polymerase, SuperFiU DNA polymerase, T7 DNA ligase. In certain embodiments, a digestion and repair reagent comprises any one or a combination of uracil DNA glycosylase (UDG), formamidopyrimidine [fapy]-DNA glycosylase (fpg), Phusion U DNA polymerase, Taq DNA polymerase, SuperFiU DNA polymerase, T4 PNK and T7 DNA ligase.

In some embodiments, methods comprise contacting a nucleic acid sample with a plurality of adaptors capable of amplification of one or more target nucleic acid sequences in the sample under conditions wherein the target nucleic acid(s) undergo a first amplification; digesting resulting first amplification products to reduce or eliminate resulting primer dimers and prepare partially digested target amplicons, thereby producing gapped, double stranded amplicons. The methods further comprise repairing the partially digested target amplicons, and purifying repaired amplicons; then amplifying the repaired target amplicons in a second amplification using universal primers, thereby producing a library of target nucleic acid sequences; and then purifying resulting library. Each of the plurality of adaptors used in the methods herein comprise a universal handle sequence and a target nucleic acid sequence and a cleavable moiety and one or more tag sequences. At least two and up to one hundred thousand target specific adaptor pairs are included in the provided methods, wherein the target nucleic acid sequence of each adaptor includes at least one cleavable moiety, the universal handle sequence does not include the cleavable moiety, and cleavable moieties are included in the flanking either end of the tag sequence. In some embodiments a digestion and repair reagent comprises any one or a combination of one or a combination of uracil DNA glycosylase (UDG). apurinic endonuclease (e.g., APE1), RecJf, formamidopyrimidine [fapy]-DNA glycosylase (fpg), Nth endonuclease III, endonuclease VIII, polynucleotide kinase (PNK), Taq DNA polymerase, DNA polymerase I and/or human DNA polymerase beta; and any one or a combination of Phusion DNA polymerase, Phusion U DNA polymerase, SuperFi DNA polymerase, Taq DNA polymerase, Human DNA polymerase beta, T4 DNA polymerase and/or T7 DNA polymerase, SuperFiU DNA and/or 9° N DNA ligase. In certain embodiments, a digestion and repair reagent comprises any one or a combination of uracil DNA glycosylase (UDG), apurinic endonuclease (e.g., APE1), Taq DNA polymerase, Phusion U DNA polymerase, SuperFiU DNA polymerase, T7 DNA ligase. In certain embodiments, a digestion and repair reagent comprises any one or a combination of uracil DNA glycosylase (UDG), formamidopyrimidine [fapy]-DNA glycosylase (fpg), Phusion U DNA polymerase, Taq DNA polymerase, SuperFiU DNA polymerase, T4 PNK and T7 DNA ligase.

In certain embodiments methods of the invention are carried out in a single, addition only workflow reaction, allowing for rapid production of highly multiplexed targeted libraries. For example, in one embodiment, methods for preparing a library of target nucleic acid sequences comprise contacting a nucleic acid sample with a plurality of adaptors capable of amplification of one or more target nucleic acid sequences in the sample under conditions wherein the target nucleic acid(s) undergo a first amplification; digesting resulting first amplification products to reduce or eliminate resulting primer dimers and prepare partially digested target amplicons, thereby producing gapped, double stranded amplicons. The methods further comprise repairing the partially digested target amplicons; then amplifying the repaired target amplicons in a second amplification using universal primers, thereby producing a library of target nucleic acid sequences, and purifying the resulting library. In certain embodiments the purification comprises a single or repeated separating step that is carried out following production of the library following the second amplification; and wherein the other method steps are conducted in a single reaction vessel without requisite transferring of a portion (aliquot) of any of the products generated in steps to another reaction vessel. Each of the plurality of adaptors used in the methods herein comprise a universal handle sequence and a target nucleic acid sequence and a cleavable moiety and optionally one or more tag sequences. At least two and up to one hundred thousand target specific adaptor pairs are included in the provided methods, wherein the target nucleic acid sequence of each adaptor includes at least one cleavable moiety and the universal handle sequence does not include the cleavable moiety. In some embodiments where an optional tag sequence is included in at least one adaptor, the cleavable moieties are included in the adaptor sequence flanking either end of the tag sequence.

In another embodiment, methods for preparing a tagged library of target nucleic acid sequences are provided comprising contacting a nucleic acid sample with a plurality of adaptors capable of amplification of one or more target nucleic acid sequences in the sample under conditions wherein the target nucleic acid(s) undergo a first amplification; digesting resulting first amplification products to reduce or eliminate resulting primer dimers and prepare partially digested target amplicons, thereby producing gapped, double stranded amplicons. The methods further comprise repairing the partially digested target amplicons; then amplifying the repaired target amplicons in a second amplification using universal primers, thereby producing a library of target nucleic acid sequences, and purifying the resulting library. In certain embodiments the purification comprises a single or repeated separating step; and wherein the other method steps are optionally conducted in a single reaction vessel without requisite transferring of a portion of any of the products generated in steps to another reaction vessel. Each of the plurality of adaptors used in the methods herein comprise a universal handle sequence and a target nucleic acid sequence and a cleavable moiety and one or more tag sequences. At least two and up to one hundred thousand target specific adaptor pairs are included in the provided methods, wherein the target nucleic acid sequence of each adaptor includes at least one cleavable moiety, the universal handle sequence does not include the cleavable moiety, and the cleavable moieties are included flanking either end of the tag sequence.

In one embodiment, methods for preparing a library of target nucleic acid sequences comprise contacting a nucleic acid sample with a plurality of adaptors capable of amplification of one or more target nucleic acid sequences in the sample under conditions wherein the target nucleic acid(s) undergo a first amplification; digesting resulting first amplification products to reduce or eliminate resulting primer dimers and prepare partially digested target amplicons, thereby producing gapped, double stranded amplicons. The methods further comprise repairing the partially digested target amplicon; then amplifying the repaired target amplicons in a second amplification using universal primers, thereby producing a library of target nucleic acid sequences, and purifying the resulting library.

In some embodiments a digestion reagent comprises any one or any combination of: uracil DNA glycosylase (UDG). AP endonuclease (APE1), RecJf, formamidopyrimidine [fapy]-DNA glycosylase (fpg), Nth endonuclease III, endonuclease VIII, polynucleotide kinase, Taq DNA polymerase, DNA polymerase I and/or human DNA polymerase beta. In certain embodiments a digestion reagent comprises any one or any combination of: uracil DNA glycosylase (UDG). AP endonuclease (APE1), RecJf, formamidopyrimidine [fapy]-DNA glycosylase (fpg), Nth endonuclease III, endonuclease VIII, polynucleotide kinase, Taq DNA polymerase, DNA polymerase I and/or human DNA polymerase beta wherein the digestion reagent lacks formamidopyrimidine [fapy]-DNA glycosylase (fpg).

In some embodiments a digestion reagent comprises a single-stranded DNA exonuclease that degrades in a 5′-3′ direction. In some embodiments a cleavage reagent comprises a single-stranded DNA exonuclease that degrades abasic sites. In some embodiments herein the digestions reagent comprises an RecJf exonuclease. In particular embodiments a digestion reagent comprises APE1 and RecJf, wherein the cleavage reagent comprises an apurinic/apyrimidinic endonuclease. In certain embodiments the digestion reagent comprises an AP endonuclease (APE1).

In some embodiments a repair reagent comprises at least one DNA polymerase; wherein the gap-filling reagent comprises: any one or any combination of: Phusion DNA polymerase, Phusion U DNA polymerase, SuperFi DNA polymerase, Taq DNA polymerase, Human DNA polymerase beta, T4 DNA polymerase and/or T7 DNA polymerase and/or SuperFi U DNA polymerase. In some embodiments a repair reagent further comprises a plurality of nucleotides.

In some embodiment a repair reagent comprises an ATP-dependent or an ATP-independent ligase; wherein the repair reagent comprises any one or any combination of: E. coli DNA ligase, T3 DNA ligase, T4 DNA ligase, T7 DNA ligase, Taq DNA ligase, 9° N DNA ligase

In certain embodiments a digestion and repair reagent comprises any one or a combination of one or a combination of uracil DNA glycosylase (UDG). apurinic endonuclease (e.g., APE1), RecJf, formamidopyrimidine [fapy]-DNA glycosylase (fpg), Nth endonuclease III, endonuclease VIII, polynucleotide kinase (PNK), Taq DNA polymerase, DNA polymerase I and/or human DNA polymerase beta; and any one or a combination of Phusion DNA polymerase, Phusion U DNA polymerase, SuperFi DNA polymerase, Taq DNA polymerase, Human DNA polymerase beta, T4 DNA polymerase and/or T7 DNA polymerase, SuperFiU DNA polymerase, E. coli DNA ligase, T3 DNA ligase, T4 DNA ligase, T7 DNA ligase, Taq DNA ligase, and/or 9° N DNA ligase. In particular embodiments, a digestion and repair reagent comprises any one or a combination of uracil DNA glycosylase (UDG), apurinic endonuclease (e.g., APE1), Taq DNA polymerase, Phusion U DNA polymerase, SuperFiU DNA polymerase, T7 DNA ligase. In certain embodiments a purification comprises a single or repeated separating step that is carried out following production of the library following the second amplification; and wherein method steps are conducted in a single reaction vessel without requisite transferring of a portion of any of the products generated in steps to another reaction vessel until a first purification. Each of the plurality of adaptors used in the methods herein comprise a universal handle sequence and a target nucleic acid sequence and a cleavable moiety and optionally one or more tag sequences. At least two and up to one hundred thousand target specific adaptor pairs are included in the provided methods, wherein the target nucleic acid sequence of each adaptor includes at least one cleavable moiety and the universal handle sequence does not include the cleavable moiety. In some embodiments where an optional tag sequence is included in at least one adaptor, the cleavable moieties are included in the adaptor sequence flanking either end of the tag sequence.

In another embodiment, methods for preparing a tagged library of target nucleic acid sequences are provided comprising contacting a nucleic acid sample with a plurality of adaptors capable of amplification of one or more target nucleic acid sequences in the sample under conditions wherein the target nucleic acid(s) undergo a first amplification; digesting resulting first amplification products to reduce or eliminate resulting primer dimers and prepare partially digested target amplicons, thereby producing gapped, double stranded amplicons. The methods further comprise repairing the partially digested target amplicons; then amplifying the repaired target amplicons in a second amplification using universal primers, thereby producing a library of target nucleic acid sequences, and purifying the resulting library. In certain embodiments a digestion and repair reagent comprises any one or a combination of one or a combination of uracil DNA glycosylase (UDG). apurinic endonuclease (e.g., APE1), RecJf, formamidopyrimidine [fapy]-DNA glycosylase (fpg), Nth endonuclease III, endonuclease VIII, polynucleotide kinase (PNK), Taq DNA polymerase, DNA polymerase I and/or human DNA polymerase beta; and any one or a combination of Phusion DNA polymerase, Phusion U DNA polymerase, SuperFi DNA polymerase, Taq DNA polymerase, Human DNA polymerase beta, T4 DNA polymerase and/or T7 DNA polymerase, SuperFiU DNA polymerase, E. coli DNA ligase, T3 DNA ligase, T4 DNA ligase, T7 DNA ligase, Taq DNA ligase, and/or 9° N DNA ligase. In particular embodiments, a digestion and repair reagent comprises any one or a combination of uracil DNA glycosylase (UDG), apurinic endonuclease (e.g., APE1), Taq DNA polymerase, Phusion U DNA polymerase, SuperFiU DNA polymerase, T7 DNA ligase. In certain embodiments the purification comprises a single or repeated separating step that is carried out following production of the library following the second amplification; and wherein steps the other method steps are conducted in a single reaction vessel without requisite transferring of a portion (aliquot) of any of the products generated in steps to another reaction vessel. Each of the plurality of adaptors used in the methods herein comprise a universal handle sequence and a target nucleic acid sequence and a cleavable moiety and one or more tag sequences. At least two and up to one hundred thousand target specific adaptor pairs are included in the provided methods, wherein the target nucleic acid sequence of each adaptor includes at least one cleavable moiety, the universal handle sequence does not include the cleavable moiety, and the cleavable moieties are included flanking either end of the tag sequence.

In some embodiments, adaptor-dimer byproducts resulting from the first amplification of step of the methods are largely removed from the resulting library. In certain embodiments the enriched population of amplified target nucleic acids contains a reduced amount of adaptor-dimer byproduct. In particular embodiments adaptor dimer byproducts are eliminated.

In some embodiments, the library is prepared in less than 4 hours. In some embodiments, the library is prepared, enriched and sequenced in less than 3 hours. In some embodiments, the library is prepared, enriched and sequenced in 2 to 3 hours. In some embodiments, the library is prepared in approximately 2.5 hours. In some embodiments, the library is prepared in approximately 2.75 hours. In some embodiments, the library is prepared in approximately 3 hours.

### Compositions

Additional aspects of the invention comprise composition comprising a plurality of nucleic acid adaptors, as well as library compositions prepared according to the methods of the invention. Provided compositions are useful in conjunction with the methods described herein as well as for additional analysis and applications known in the art.

Thus, provided are composition comprising a plurality of nucleic acid adaptors, wherein each of the plurality of adaptors comprises a 5′ universal handle sequence, optionally one or more tag sequences, and a 3′ target nucleic acid sequence wherein each adaptor comprises a cleavable moiety, wherein the target nucleic acid sequence of the adaptor includes at least one cleavable moiety, and when tag sequences are present cleavable moieties are included flanking either end of the tag sequence and wherein the universal handle sequence does not include the cleavable moiety. At least two and up to one hundred thousand target specific adaptor pairs are included in provided compositions. Provided composition allow for rapid production of highly multiplexed targeted libraries.

In some embodiments, provided compositions comprise plurality of nucleic acid adaptors, wherein each of the plurality of adaptors comprise a 5′ universal handle sequence, one or more tag sequences, and a 3′ target nucleic acid sequence wherein each adaptor comprises a cleavable moiety; wherein the target nucleic acid sequence of the adaptor includes at least one cleavable moiety, cleavable moieties are included flanking either end of the tag sequence and the universal handle sequence does not include the cleavable moiety. At least two and up to one hundred thousand target specific adaptor pairs are included in provided compositions. Provided composition allow for rapid production of highly multiplexed, tagged, targeted libraries.

Primer/adaptor compositions may be single stranded or double stranded. In some embodiments adaptor compositions comprise are single stranded adaptors. In some embodiments adaptor compositions comprise double stranded adaptors. In some embodiments adaptor compositions comprise a mixture of single stranded and double stranded adaptors.

In some embodiments, compositions include a plurality of adaptors capable of amplification of one or more target nucleic acid sequences comprising a multiplex of adaptor pairs capable of amplification of at least two different target nucleic acid sequences wherein the target-specific primer sequence is substantially non-complementary to other target specific primer sequences in the composition. In some embodiments, the composition comprises at least 25, 50, 75, 100, 150, 200, 250, 300, 350, 400, 450, 500, 750, 1000, 1250, 1500, 1750, 2000, 2250, 2500, 2750, 3000, 3250, 3500, 3750, 4000, 4500, 5000, 5500, 6000, 7000, 8000, 9000, 10000, 11000, or 12000, or more target-specific adaptor pairs. In some embodiments, target-specific adaptor pairs comprise about 15 nucleotides to about 40 nucleotides in length, wherein at least one nucleotide is replaced with a cleavable group. In some embodiments the cleavable group is a uridine nucleotide. In some embodiments, the target-specific adaptor pairs are designed to amplify an exon, gene, exome or region of the genome associated with a clinical or pathological condition, e.g., amplification of one or more sites comprising one or more mutations (e.g., driver mutation) associated with a cancer, e.g., lung, colon, breast cancer, etc., or amplification of mutations associated with an inherited disease, e.g., cystic fibrosis, muscular dystrophies, etc. In some embodiments, the target-specific adaptor pairs when hybridized to a target sequence and amplified as provided herein generates a library of adaptor-ligated amplified target sequences that are about 100 to about 600 base pairs in length. In some embodiments, no one adaptor-ligated amplified target sequence is overexpressed in the library by more than 30% as compared to the remainder of other adaptor-ligated amplified target sequences in the library. In some embodiments, an adaptor-ligated amplified target sequence library is substantially homogenous with respect to GC content, amplified target sequence length or melting temperature (Tm) of the respective target sequences.

In some embodiments, the target-specific primer sequences of adaptor pairs in the compositions of the invention are target-specific sequences that can amplify specific regions of a nucleic acid molecule. In some embodiments, the target-specific adaptors can amplify genomic DNA or cDNA. In some embodiments, target-specific adaptors can amplify mammalian nucleic acid, such as, but not limited to human DNA or RNA, murine DNA or RNA, bovine DNA or RNA, canine DNA or RNA, equine DNA or RNA, or any other mammal of interest. In other embodiments, target specific adaptors include sequences directed to amplify plant nucleic acids of interest. In other embodiments, target specific adaptors include sequences directed to amplify infectious agents, e.g., bacterial and/or viral nucleic acids. In some embodiments, the amount of nucleic acid required for selective amplification is from about 1 ng to 1 microgram. In some embodiments, the amount of nucleic acid required for selective amplification of one or more target sequences is about 1 ng, about 5 ng or about 10 ng. In some embodiments, the amount of nucleic acid required for selective amplification of target sequence is about 10 ng to about 200 ng.

As described herein, each of the plurality of adaptors comprises a 5′ universal handle sequence. In some embodiments a universal handle sequence comprises any one or any combination of an amplification primer binding sequence, a sequencing primer binding sequence and/or a capture primer binding sequence. In some embodiments the comparable maximal minimum melting temperatures of each adaptor universal handle sequence is higher than the comparable maximal minimum melting temperatures of each target nucleic acid sequence and each tag sequence present in the same adaptor. Preferably, the universal handle sequences of provided adaptors do not exhibit significant complementarity and/or hybridization to any portion of a unique tag sequence and/or target nucleic acid sequence of interest. In some embodiments a first universal handle sequence comprises any one or any combination of an amplification primer binding sequence, a sequencing primer binding sequence and/or a capture primer binding sequence. In some embodiments a second universal handle sequence comprises any one or any combination of an amplification primer binding sequence, a sequencing primer binding sequence and/or a capture primer binding sequence. In certain embodiments first and second universal handle sequences correspond to forward and reverse universal handle sequences and in certain embodiments the same first and second universal handle sequences are included for each of the plurality of target specific adaptor pairs. Such forward and reverse universal handle sequences are targeted in conjunction with universal primers to carry out a second amplification of repaired amplicons in production of libraries according to methods of the invention. In certain embodiments a first 5′ universal handle sequence comprises two universal handle sequences (e.g., a combination of an amplification primer binding sequence, a sequencing primer binding sequence and/or a capture primer binding sequence); and a second 5′ universal sequence comprises two universal handle sequences (e.g., a combination of an amplification primer binding sequence, a sequencing primer binding sequence and/or a capture primer binding sequence), wherein the 5′ first and second universal handle sequences do not exhibit significant hybridization to any portion of a target nucleic acid sequence of interest.

The structure and properties of universal amplification primers or universal primers are well known to those skilled in the art and can be implemented for utilization in conjunction with provided methods and compositions to adapt to specific analysis platforms. Universal handle sequences of the adaptors provided herein are adapted accordingly to accommodate a preferred universal primer sequences. For example, e.g., as described herein universal P1 and A primers with optional barcode sequences have been described in the art and utilized for sequencing on Ion Torrent sequencing platforms (Ion Xpress™ Adapters. Thermo Fisher Scientific). Similarly, additional and other universal adaptor/primer sequences described and known in the art (e.g., Illumina universal adaptor/primer sequences can be found, e.g., at //support.illumina.com/content/dam/illumina-support/documents/documentation/chemistry_documentation/experiment-design/illumina-adapter-sequences_1000000002694-01.pdf: PacBio universal adaptor/primer sequences, can be found. e.g., at //s3.amazonaws.com/files.pacb.com/pdf/Guide_Pacific_Biosciences_Template_Preparation_and_Sequencing.pdf; etc.) can be used in conjunction with the methods and compositions provided herein. Suitable universal primers of appropriate nucleotide sequence for use with adaptors of the invention are readily prepared using standard automated nucleic acid synthesis equipment and reagents in routine use in the art. One single type of universal primer or separate types (or even a mixture) of two different universal primers, for example a pair of universal amplification primers suitable for amplification of repaired amplicons in a second amplification are included for use in the methods of the invention. Universal primers optionally include a different tag (barcode) sequence, where the tag (barcode) sequence does not hybridize to the adaptor. Barcode sequences incorporated into amplicons in a second universal amplification can be utilized e.g., for effective identification of sample source.

In some embodiments adaptors further comprise a unique tag sequence located between the 5′ first universal handle sequence and the 3′ target-specific sequence, and wherein the unique tag sequence does not exhibit significant complementarity and/or hybridization to any portion of a unique tag sequence and/or target nucleic acid sequence of interest. In some embodiments the plurality of primer adaptor pairs has 10⁴-10⁹ different tag sequence combinations. Thus in certain embodiments each generated target specific adaptor pair comprises 10⁴-10⁹ different tag sequences. In some embodiments the plurality of primer adaptors comprise each target specific adaptor comprising at least 1 different unique tag sequence and up to 10⁵ different unique tag sequences. In some embodiments the plurality of primer adaptors comprise each target specific adaptor comprising at least 1 different unique tag sequence and up to 10⁵ different unique tag sequences. In certain embodiments each generated target specific amplicon generated comprises at least two and up to 10⁹ different adaptor combinations comprising different tag sequences, each having two different unique tag sequences. In some embodiments the plurality of primer adaptors comprise each target specific adaptor comprising 4096 different tag sequences. In certain embodiments each generated target specific amplicon generated comprises up to 16,777,216 different adaptor combinations comprising different tag sequences, each having two different unique tag sequences.

In some embodiments individual primer adaptors in the plurality of adaptors include a unique tag sequence (e.g., contained in a tag adaptor) comprising different random tag sequences alternating with fixed tag sequences. In some embodiments, the at least one unique tag sequence comprises a at least one random sequence and at least one fixed sequence, or comprises a random sequence flanked on both sides by a fixed sequence, or comprises a fixed sequence flanked on both sides by a random sequence. In some embodiments a unique tag sequence includes a fixed sequence that is 2-2000 nucleotides or base-pairs in length. In some embodiments a unique tag sequence includes a random sequence that is 2-2000 nucleotides or base-pairs in length.

In some embodiments, unique tag sequences include a sequence having at least one random sequence interspersed with fixed sequences. In some embodiments, individual tag sequences in a plurality of unique tags have the structure (N)ₙ(X)ₓ(M)ₘ(Y)_{y}, wherein “N” represents a random tag sequence that is generated from A, G, C, T, U or I, and wherein “n” is 2-10 which represents the nucleotide length of the “N” random tag sequence; wherein “X” represents a fixed tag sequence, and wherein “x” is 2-10 which represents the nucleotide length of the “X” random tag sequence; wherein “M” represents a random tag sequence that is generated from A, G, C, T, U or I, wherein the random tag sequence “M” differs or is the same as the random tag sequence “N”, and wherein “m” is 2-10 which represents the nucleotide length of the “f” random tag sequence; and wherein “Y” represents a fixed tag sequence, wherein the fixed tag sequence of “Y” is the same or differs from the fixed tag sequence of “X”, and wherein “y” is 2-10 which represents the nucleotide length of the “Y” random tag sequence. In some embodiments, the fixed tag sequence “X” is the same in a plurality of tags. In some embodiments, the fixed tag sequence “X” is different in a plurality of tags. In some embodiments, the fixed tag sequence “Y” is the same in a plurality of tags. In some embodiments, the fixed tag sequence “Y” is different in a plurality of tags. In some embodiments, the fixed tag sequences “(X)ₓ” and “(Y)_{y}” within the plurality of adaptors are sequence alignment anchors.

In some embodiments, the random sequence within a unique tag sequence is represented by “N”, and the fixed sequence is represented by “X”. Thus, a unique tag sequence is represented by N₁N₂N₃X₁X₂X₃ or by N₁N₂N₃X₁X₂X₃N₄N₅N₆X₄X₅X₆. Optionally, a unique tag sequence can have a random sequence in which some or all of the nucleotide positions are randomly selected from a group consisting of A, G, C, T, U and I. For example, a nucleotide for each position within a random sequence is independently selected from any one of A, G, C, T, U or I, or is selected from a subset of these six different types of nucleotides. Optionally, a nucleotide for each position within a random sequence is independently selected from any one of A, G, C or T. In some embodiments, the first fixed tag sequence “X₁X₂X₃” is the same or different sequence in a plurality of tags. In some embodiments, the second fixed tag sequence “X₄X₅X₆” is the same or different sequence in a plurality of tags. In some embodiments, the first fixed tag sequence “X₁X₂X₃” and the second fixed tag sequence “X₄X₅X₆” within the plurality of adaptors are sequence alignment anchors.

In some embodiments, a unique tag sequence comprises the sequence 5′-NNNACTNNNTGA-3′, where “N” represents a position within the random sequence that is generated randomly from A, G, C or T, the number of possible distinct random tags is calculated to be 4⁶ (or 4\hat{ }6) is about 4096, and the number of possible different combinations of two unique tags is 4¹² (or 4\hat{ }12) is about 16.78 million. In some embodiments, the underlined portions of 5′-NNNACTNNNTGA-3′ are a sequence alignment anchor.

In some embodiments, the fixed sequences within the unique tag sequence is a sequence alignment anchor that can be used to generate error-corrected sequencing data. In some embodiments fixed sequences within the unique tag sequence is a sequence alignment anchor that can be used to generate a family of error-corrected sequencing reads.

Adaptors provided herein comprise at least one cleavable moiety. In some embodiments a cleavable moiety is within the 3′ target-specific sequence. In some embodiments a cleavable moiety is at or near the junction between the 5′ first universal handle sequence and the 3′ target-specific sequence. In some embodiments a cleavable moiety is at or near the junction between the 5′ first universal handle sequence and the unique tag sequence, and at or near the junction between the unique tag sequence and the 3′ target-specific sequence. The cleavable moiety can be present in a modified nucleotide, nucleoside or nucleobase. In some embodiments, the cleavable moiety can include a nucleobase not naturally occurring in the target sequence of interest.

In some embodiments the at least one cleavable moiety in the plurality of adaptors is a uracil base, uridine or a deoxyuridine nucleotide. In some embodiments a cleavable moiety is within the 3′ target-specific sequence and the junctions between the 5′ universal handle sequence and the unique tag sequence and/or the 3′ target specific sequence wherein the at least one cleavable moiety in the plurality of adaptors is cleavable with uracil DNA glycosylase (UDG). In some embodiments, a cleavable moiety is cleaved, resulting in a susceptible abasic site, wherein at least one enzyme capable of reacting on the abasic site generates a gap comprising an extendible 3′ end. In certain embodiments the resulting gap comprises a 5′-deoxyribose phosphate group. In certain embodiments the resulting gap comprises an extendible 3′ end and a 5′ ligatable phosphate group.

In another embodiment, inosine can be incorporated into a DNA-based nucleic acid as a cleavable group. In one exemplary embodiment, EndoV can be used to cleave near the inosine residue. In another exemplary embodiment, the enzyme hAAG can be used to cleave inosine residues from a nucleic acid creating abasic sites.

Where a cleavable moiety is present, the location of the at least one cleavable moiety in the adaptors does not significantly change the melting temperature (Tm) of any given double-stranded adaptor in the plurality of double-stranded adaptors. The melting temperatures (Tm) of any two given double-stranded adaptors from the plurality of double-stranded adaptors are substantially the same, wherein the melting temperatures (Tm) of any two given double-stranded adaptors does not differ by more than 10° C. of each other. However, within each of the plurality of adaptors, the melting temperatures of sequence regions differs, such that the comparable maximal minimum melting temperature of, for example, the universal handle sequence, is higher than the comparable maximal minimum melting temperatures of either the unique tag sequence and/or the target specific sequence of any adaptor. This localized differential in comparable maximal minimum melting temperatures can be adjusted to optimize digestion and repair of amplicons and ultimately improved effectiveness of the methods provided herein.

Further provided are compositions comprising a nucleic acid library generated by methods of the invention. Thus, provided are composition comprising a plurality of amplified target nucleic acid amplicons, wherein each of the plurality of amplicons comprises a 5′ universal handle sequence, optionally a first unique tag sequences, an intermediate target nucleic acid sequence, optionally a second unique tag sequences and a 3′ universal handle sequence. At least two and up to one hundred thousand target specific amplicons are included in provided compositions. Provided compositions include highly multiplexed targeted libraries. In some embodiments, provided compositions comprise a plurality of nucleic acid amplicons, wherein each of the plurality of amplicons comprise a a 5′ universal handle sequence, a first unique tag sequences, an intermediate target nucleic acid sequence, a second unique tag sequences and a 3′ universal handle sequence. At least two and up to one hundred thousand target specific tagged amplicons are included in provided compositions. Provided compositions include highly multiplexed tagged targeted libraries.

In some embodiments, library compositions include a plurality of target specific amplicons comprising a multiplex of at least two different target nucleic acid sequences. In some embodiments, the composition comprises at least 25, 50, 75, 100, 150, 200, 250, 300, 350, 400, 450, 500, 750, 1000, 1250, 1500, 1750, 2000, 2250, 2500, 2750, 3000, 3250, 3500, 3750, 4000, 4500, 5000, 5500, 6000, 7000, 8000, 9000, 10000, 11000, or 12000, or more target-specific amplicons. In some embodiments, the target-specific amplicons comprise one or more exon, gene, exome or region of the genome associated with a clinical or pathological condition, e.g., amplicons comprising one or more sites comprising one or more mutations (e.g., driver mutation) associated with a cancer, e.g., lung, colon, breast cancer, etc., or amplicons comprising mutations associated with an inherited disease, e.g., cystic fibrosis, muscular dystrophies, etc. In some embodiments, the target-specific amplicons comprise a library of adaptor-ligated amplicon target sequences that are about 100 to about 750 base pairs in length.

As described herein, each of the plurality of amplicons comprises a 5′ universal handle sequence. In some embodiments a universal handle sequence comprises any one or any combination of an amplification primer binding sequence, a sequencing primer binding sequence and/or a capture primer binding sequence. Preferably, the universal handle sequences of provided adaptors do not exhibit significant complementarity and/or hybridization to any portion of a unique tag sequence and/or target nucleic acid sequence of interest. In some embodiments a first universal handle sequence comprises any one or any combination of an amplification primer binding sequence, a sequencing primer binding sequence and/or a capture primer binding sequence. In some embodiments a second universal handle sequence comprises any one or any combination of an amplification primer binding sequence, a sequencing primer binding sequence and/or a capture primer binding sequence. In certain embodiments first and second universal handle sequences correspond to forward and reverse universal handle sequences and in certain embodiments the same first and second universal handle sequences are included for each of the plurality of target specific amplicons. Such forward and reverse universal handle sequences are targeted in conjunction with universal primers to carry out a second amplification of a preliminary library composition in production of resulting amplified according to methods of the invention. In certain embodiments a first 5′ universal handle sequence comprises two universal handle sequences (e.g., a combination of an amplification primer binding sequence, a sequencing primer binding sequence and/or a capture primer binding sequence); and a second 5′ universal sequence comprises two universal handle sequences (e.g., a combination of an amplification primer binding sequence, a sequencing primer binding sequence and/or a capture primer binding sequence), wherein the 5′ first and second universal handle sequences do not exhibit significant hybridization to any portion of a target nucleic acid sequence of interest.

The structure and properties of universal amplification primers or universal primers are well known to those skilled in the art and can be implemented for utilization in conjunction with provided methods and compositions to adapt to specific analysis platforms. Universal handle sequences of the adaptors and amplicons provided herein are adapted accordingly to accommodate a preferred universal primer sequences. For example, e.g., as described herein universal P1 and A primers with optional barcode sequences have been described in the art and utilized for sequencing on Ion Torrent sequencing platforms (Ion Xpress™ Adapters, Thermo Fisher Scientific). Similarly, additional and other universal adaptor/primer sequences described and known in the art (e.g., Illumina universal adaptor/primer sequences can be found, e.g., at //support.illumina.com/content/dam/illumina-support/documents/documentation/chemistry_documentation/experiment-design/illumina-adapter-sequences_1000000002694-01.pdf, PacBio universal adaptor/primer sequences, can be found, e.g., at //s3.amazonaws.com/files.pacb.com/pdf/Guide_Pacific_Biosciences_Template_Preparation_and_Seq uencing.pdf; etc.) can be used in conjunction with the methods and compositions provided herein. Suitable universal primers of appropriate nucleotide sequence for use with libraries of the invention are readily prepared using standard automated nucleic acid synthesis equipment and reagents in routine use in the art. One single type or separate types (or even a mixture) of two different universal primers, for example a pair of universal amplification primers suitable for amplification of a preliminary library may be used in production of the libraries of the invention. Universal primers optionally include a tag (barcode) sequence, where the tag (barcode) sequence does not hybridize to adaptor sequence or to target nucleic acid sequences. Barcode sequences incorporated into amplicons in a second universal amplification can be utilized e.g., for effective identification of sample source to thereby generate a barcoded library. Thus provided compositions include highly multiplexed barcoded targeted libraries. Provided compositions also include highly multiplexed barcoded tagged targeted libraries.

In some embodiments amplicon libraries comprise a unique tag sequence located between the 5′ first universal handle sequence and the 3′ target-specific sequence, and wherein the unique tag sequence does not exhibit significant complementarity and/or hybridization to any portion of a unique tag sequence and/or target nucleic acid sequence. In some embodiments the plurality of amplicons has 10⁴-10⁹ different tag sequence combinations. Thus in certain embodiments each of the plurality of amplicons in a library comprises 10⁴-10⁹ different tag sequences. In some embodiments each of the plurality of amplicons in a library comprises at least 1 different unique tag sequence and up to 10⁵ different unique tag sequences. In certain embodiments each target specific amplicon in a library comprises at least two and up to 10⁹ different combinations comprising different tag sequences, each having two different unique tag sequences. In some embodiments each of the plurality of amplicons in a library comprise a tag sequence comprising 4096 different tag sequences. In certain embodiments each target specific amplicon of a library comprises up to 16,777,216 different combinations comprising different tag sequences, each having two different unique tag sequences.

In some embodiments individual amplicons in the plurality of amplicons of a library include a unique tag sequence (e.g., contained in a tag adaptor sequence) comprising different random tag sequences alternating with fixed tag sequences. In some embodiments, the at least one unique tag sequence comprises a at least one random sequence and at least one fixed sequence, or comprises a random sequence flanked on both sides by a fixed sequence, or comprises a fixed sequence flanked on both sides by a random sequence. In some embodiments a unique tag sequence includes a fixed sequence that is 2-2000 nucleotides or base-pairs in length. In some embodiments a unique tag sequence includes a random sequence that is 2-2000 nucleotides or base-pairs in length.

In some embodiments, unique tag sequences include a sequence having at least one random sequence interspersed with fixed sequences. In some embodiments, individual tag sequences in a plurality of unique tags have the structure (N)ₙ(X)ₓ(M)ₘ(Y)_{y}, wherein “N” represents a random tag sequence that is generated from A, G, C, T, U or I, and wherein “n” is 2-10 which represents the nucleotide length of the “N” random tag sequence; wherein “X” represents a fixed tag sequence, and wherein “x” is 2-10 which represents the nucleotide length of the “X” random tag sequence; wherein “M” represents a random tag sequence that is generated from A, G, C, T, U or I, wherein the random tag sequence “M” differs or is the same as the random tag sequence “N”, and wherein “m” is 2-10 which represents the nucleotide length of the “M” random tag sequence; and wherein “Y” represents a fixed tag sequence, wherein the fixed tag sequence of “Y” is the same or differs from the fixed tag sequence of “X”, and wherein “y” is 2-10 which represents the nucleotide length of the “Y” random tag sequence. In some embodiments, the fixed tag sequence “X” is the same in a plurality of tags. In some embodiments, the fixed tag sequence “X” is different in a plurality of tags. In some embodiments, the fixed tag sequence “Y” is the same in a plurality of tags. In some embodiments, the fixed tag sequence “Y” is different in a plurality of tags. In some embodiments, the fixed tag sequences “(X)ₓ” and “(Y)_{y}” within the plurality of amplicons are sequence alignment anchors.

In some embodiments, the random sequence within a unique tag sequence is represented by “N”, and the fixed sequence is represented by “X”. Thus, a unique tag sequence is represented by N₁N₂N₃X₁X₂X₃ or by N₁N₂N₃X₁X₂X₃N₄N₅N₆X₄X₅X₆. Optionally, a unique tag sequence can have a random sequence in which some or all of the nucleotide positions are randomly selected from a group consisting of A, G, C, T, U and I. For example, a nucleotide for each position within a random sequence is independently selected from any one of A, G, C, T, U or I, or is selected from a subset of these six different types of nucleotides. Optionally, a nucleotide for each position within a random sequence is independently selected from any one of A, G, C or T. In some embodiments, the first fixed tag sequence “X₁X₂X₃” is the same or different sequence in a plurality of tags. In some embodiments, the second fixed tag sequence “X₄X₅X₆” is the same or different sequence in a plurality of tags. In some embodiments, the first fixed tag sequence “X₁X₂X₃” and the second fixed tag sequence “X₄X₅X₆” within the plurality of amplicons are sequence alignment anchors.

In some embodiments, a unique tag sequence comprises the sequence 5′-NNNACTNNNTGA-3′, where “N” represents a position within the random sequence that is generated randomly from A, G, C or T, the number of possible distinct random tags is calculated to be 4⁶ (or 4\hat{ }6) is about 4096, and the number of possible different combinations of two unique tags is 4¹² (or 4\hat{ }12) is about 16.78 million. In some embodiments, the underlined portions of 5′-NNNACTNNNTGA-3′ are a sequence alignment anchor.

In some embodiments, the fixed sequences within the unique tag sequence is a sequence alignment anchor that can be used to generate error-corrected sequencing data. In some embodiments fixed sequences within the unique tag sequence is a sequence alignment anchor that can be used to generate a family of error-corrected sequencing reads.

### Kits, Systems

Further provided herein are kits for use in preparing libraries of target nucleic acids using methods of the first or second aspects of the invention. Embodiments of a kit comprise a supply of at least a pair of target specific adaptors as defined herein which are capable of producing a first amplification product; as well as optionally a supply of at least one universal pair of amplification primers capable of annealing to the universal handle(s) of the adaptor and priming synthesis of an amplification product, which amplification product would include a target sequence of interest ligated to a universal sequence. Adaptors and/or primers may be supplied in kits ready for use, or more preferably as concentrates requiring dilution before use, or even in a lyophilized or dried form requiring reconstitution prior to use. In certain embodiments kits further include a supply of a suitable diluent for dilution or reconstitution of the components. Optionally, kits further comprise supplies of reagents, buffers, enzymes, dNTPs, etc., for use in carrying out amplification, digestion, repair, and/or purification in the generation of library as provided herein. Non-limiting examples of such reagents are as described in the Materials and Methods sections of the accompanying Exemplification. Further components which optionally are supplied in the kit include components suitable for purification of libraries prepared using the provided methods. In some embodiments, provided is a kit for generating a target-specific library comprising a plurality of target-specific adaptors having a 5′ universal handle sequence, a 3′ target specific sequence and a cleavable group, a DNA polymerase, an adaptor, dATP, dCTP, dGTP, dTTP, and a digestion reagent. In some embodiments, the kit further comprises one or more antibodies, a repair reagent, universal primers optionally comprising nucleic acid barcodes, purification solutions or columns.

Particular features of adaptors for inclusion in kits are as described elsewhere herein in relation to other aspects of the invention. The structure and properties of universal amplification primers are well known to those skilled in the art and can be implemented for utilization in conjunction with provided methods and compositions to adapt to specific analysis platforms (e.g., as described herein universal P1 and A primers have been described in the art and utilized for sequencing on Ion Torrent sequencing platforms). Similarly, additional and other universal adaptor/primer sequences described and known in the art (e.g., Illumina universal adaptor/primer sequences, PacBio universal adaptor/primer sequences, etc.) can be used in conjunction with the methods and compositions provided herein. Suitable primers of appropriate nucleotide sequence for use with adaptors included in the kit is readily prepared using standard automated nucleic acid synthesis equipment and reagents in routine use in the art. A kit may include a supply of one single type of universal primer or separate types (or even a mixture) of two different universal primers, for example a pair of amplification primers suitable for amplification of templates modified with adaptors in a first amplification. A kit may comprise at least a pair of adaptors for first amplification of a sample of interest according to the methods of the invention, plus at least two different amplification primers that optionally carry a different tag (barcode) sequence, where the tag (barcode) sequence does not hybridize to the adaptor. A kit can be used to amplify at least two different samples where each sample is amplified according to methods of the invention separately and a second amplification comprises using a single universal primer having a barcode, and then pooling prepared sample libraries after library preparations. In some embodiments a kit includes different universal primer-pairs for use in second amplification step described herein. In this context the ‘universal’ primer-pairs may be of substantially identical nucleotide sequence but differ with respect to some other feature or modification.

Further provided are systems, e.g., systems used to practice methods provided herein, and/or comprising compositions provided herein. In some embodiments, systems facilitate methods carried out in automated mode. In certain embodiments, systems facilitate high throughput mode. In certain embodiments, systems include, e.g., a fluid handling element, a fluid containing element, a heat source and/or heat sink for achieving and maintaining a desired reaction temperature, and/or a robotic element capable of moving components of the system from place to place as needed (e.g., a multiwell plate handling element).

### Samples

As defined herein, “sample” and its derivatives, is used in its broadest sense and includes any specimen, culture and/or the like that is suspected of including a target nucleic acid. In some embodiments, a sample comprises DNA, RNA, chimeric nucleic acid, hybrid nucleic acid, multiplex-forms of nucleic acids or any combination of two or more of the foregoing. In some embodiments a sample useful in conjunction with methods of the invention includes any biological, clinical, surgical, agricultural, atmospheric or aquatic-based specimen containing one or more target nucleic acid of interest. In some embodiments, a sample includes nucleic acid molecules obtained from an animal such as a human or mammalian source. In another embodiment, a sample includes nucleic acid molecules obtained from a non-mammalian source such as a plant, bacteria, virus or fungus. In some embodiments, the source of the nucleic acid molecules may be an archived or extinct sample or species. In some embodiments a sample includes isolated nucleic acid sample prepared, for example, from a source such as genomic DNA, RNA or a prepared sample such as, e.g., fresh-frozen or formalin-fixed paraffin-embedded (FFPE) nucleic acid specimen. It is also envisioned that a sample is from a single individual, a collection of nucleic acid samples from genetically related members, multiple nucleic acid samples from genetically unrelated members, multiple nucleic acid samples (matched) from a single individual such as a tumor sample and normal tissue sample, or genetic material from a single source that contains two distinct forms of genetic material such as maternal and fetal DNA obtained from a maternal subject, or the presence of contaminating bacteria DNA in a sample that contains plant or animal DNA. In some embodiments, a source of nucleic acid material includes nucleic acids obtained from a newborn (e.g., a blood sample for newborn screening). In some embodiments, provided methods comprise amplification of multiple target-specific sequences from a single nucleic acid sample. In some embodiments, provided methods comprise target-specific amplification of two or more target sequences from two or more nucleic acid samples or species. In certain embodiments, provided methods comprise amplification of highly multiplexed target nucleic acid sequences from a single sample. In particular embodiments, provided methods comprise amplification of highly multiplexed target nucleic acid sequences from more than one sample, each from the same source organism.

In some embodiments a sample comprises a mixture of target nucleic acids and non-target nucleic acids. In certain embodiments a sample comprises a plurality of initial polynucleotides which comprises a mixture of one or more target nucleic acids and may include one or more non-target nucleic acids. In some embodiments a sample comprising a plurality of polynucleotides comprises a portion or aliquot of an originating sample; in some embodiments, a sample comprises a plurality of polynucleotides which is the entire originating sample. In some embodiments a sample comprises a plurality of initial polynucleotides is isolated from the same source or from the same subject at different time points.

In some embodiments, a nucleic acid sample includes cell-free nucleic acids from a biological fluid, nucleic acids from a tissue, nucleic acids from a biopsied tissue, nucleic acids from a needle biopsy, nucleic acids from a single cell or nucleic acids from two or more cells. In certain embodiments, a single reaction mixture contains 1-100 ng of the plurality of initial polynucleotides. In some embodiments a plurality of initial polynucleotides comprises a formalin fixed paraffin-embedded (FFPE) sample; genomic DNA; RNA; cell free DNA or RNA; circulating tumor DNA or RNA; fresh frozen sample, or a mixture of two or more of the foregoing; and in some embodiments a the plurality of initial polynucleotides comprises a nucleic acid reference standard. In some embodiments, a sample includes nucleic acid molecules obtained from biopsies, tumors, scrapings, swabs, blood, mucus, urine, plasma, semen, hair, laser capture micro-dissections, surgical resections, and other clinical or laboratory obtained sample. In some embodiments, a sample is an epidemiological, agricultural, forensic or pathogenic sample. In certain embodiments, a sample includes a reference. In some embodiments a sample is a normal tissue or well documented tumor sample. In certain embodiments a reference is a standard nucleic acid sequence (e.g., Hg19).

### Target Nucleic Acid Sequence Analysis

Provided methods and compositions of the invention are particularly suitable for amplifying, optionally tagging, and preparing target sequences for subsequent analysis. Thus, in some embodiments, methods provided herein include analyzing resulting library preparations. For example, methods comprise analysis of a polynucleotide sequence of a target nucleic acid, and, where applicable, analysis of any tag sequence(s) added to a target nucleic acid. In some embodiments wherein multiple target nucleic acid regions are amplified, provided methods include determining polynucleotide sequences of multiple target nucleic acids. Provided methods further optionally include using a second tag sequence(s), e.g., barcode sequence, to identify the source of the target sequence (or to provide other information about the sample source). In certain embodiments, use of prepared library composition is provided for analysis of the sequences of the nucleic acid library.

In particular embodiments, use of prepared tagged library compositions is provided for further analyzing the sequences of the target nucleic acid library. In some embodiments determination of sequences comprises determining the abundance of at least one of the target sequences in the sample. In some embodiments determination of a low frequency allele in a sample is comprised in determination of sequences of a nucleic acid library. In certain embodiments, determination of the presence of a mutant target nucleic acid in the plurality of polynucleotides is comprised in determination of sequences of a nucleic acid library. In some embodiments, determination of the presence of a mutant target nucleic acid comprises detecting the abundance level of at least one mutant target nucleic acid in the plurality of polynucleotides. For example, such determination comprises detecting at least one mutant target nucleic acid is present at 0.05% to 1% of the original plurality of polynucleotides in the sample, detecting at least one mutant target nucleic acid is present at about 1% to about 5% of the polynucleotides in the sample, and/or detecting at least 85%-100% of target nucleic acids in sample. In some embodiments, determination of the presence of a mutant target nucleic acid comprises detecting and identification of copy number variation and/or genetic fusion sequences in a sample.

In some embodiments, nucleic acid sequencing of the amplified target sequences produced by the teachings of this disclosure include de novo sequencing or targeted re-sequencing. In some embodiments, nucleic acid sequencing further includes comparing the nucleic acid sequencing results of the amplified target sequences against a reference nucleic acid sequence. In some embodiments, nucleic acid sequencing of the target library sequences further includes determining the presence or absence of a mutation within a nucleic acid sequence. In some embodiments, nucleic acid sequencing includes the identification of genetic markers associated with disease (e.g., cancer and/or inherited disease).

In some embodiments, prepared library of target sequences of the disclosed methods is used in various downstream analysis or assays with, or without, further purification or manipulation. In some embodiments analysis comprises sequencing by traditional sequencing reactions, high throughput next generation sequencing, targeted multiplex array sequence detection, or any combination of two or more of the foregoing. In certain embodiments analysis is carried out by high throughput next generation sequencing. In particular embodiments sequencing is carried out in a bidirectional manner, thereby generating sequence reads in both forward and reverse strands for any given amplicon.

In some embodiments, library prepared according to the methods provided herein is then further manipulated for additional analysis. For example, prepared library sequences is used in downstream enrichment techniques known in the art, such a bridge amplification or emPCR to generate a template library that is then used in next generation sequencing. In some embodiments, the target nucleic acid library is used in an enrichment application and a sequencing application. For example, sequence determination of a provided target nucleic acid library is accomplished using any suitable DNA sequencing platform. In some embodiments, the library sequences of the disclosed methods or subsequently prepared template libraries is used for single nucleotide polymorphism (SNP) analysis, genotyping or epigenetic analysis, copy number variation analysis, gene expression analysis, analysis of gene mutations including but not limited to detection, prognosis and/or diagnosis, detection and analysis of rare or low frequency allele mutations, nucleic acid sequencing including but not limited to de novo sequencing, targeted resequencing and synthetic assembly analysis. In one embodiment, prepared library sequences are used to detect mutations at less than 5% allele frequency. In some embodiments, the methods disclosed herein is used to detect mutations in a population of nucleic acids at less than 4%, 3%, 2% or at about 1% allele frequency. In another embodiment, libraries prepared as described herein are sequenced to detect and/or identify germline or somatic mutations from a population of nucleic acid molecules. In certain embodiments, sequencing adaptors are ligated to the ends of the prepared libraries generate a plurality of libraries suitable for nucleic acid sequencing.

In some embodiments, methods for preparing a target-specific amplicon library are provided for use in a variety of downstream processes or assays such as nucleic acid sequencing or clonal amplification. In some embodiments, the library is amplified using bridge amplification or emPCR to generate a plurality of clonal templates suitable for nucleic acid sequencing. For example, optionally following target-specific amplification a secondary and/or tertiary amplification process including, but not limited to, a library amplification step and/or a clonal amplification step is performed. “Clonal amplification” refers to the generation of many copies of an individual molecule. Various methods known in the art is used for clonal amplification. For example, emulsion PCR is one method, and involves isolating individual DNA molecules along with primer-coated beads in aqueous bubbles within an oil phase. A polymerase chain reaction (PCR) then coats each bead with clonal copies of the isolated library molecule and these beads are subsequently immobilized for later sequencing. Emulsion PCR is used in the methods published by Marguilis et al. and Shendure and Porreca et al. (also known as “polony sequencing”, commercialized by Agencourt and recently acquired by Applied Biosystems). Margulies, et al. (2005) Nature 437: 376-380; Shendure et al., Science 309 (5741): 1728-1732. Another method for clonal amplification is “bridge PCR,” where fragments are amplified upon primers attached to a solid surface. These methods, as well as other methods of clonal amplification, both produce many physically isolated locations that each contain many copies derived from a single molecule polynucleotide fragment. Thus, in some embodiments, the one or more target specific amplicons are amplified using for example, bridge amplification or emPCR to generate a plurality of clonal templates suitable for nucleic acid sequencing.

In some embodiments, at least one of the library sequences to be clonally amplified are attached to a support or particle. A support can be comprised of any suitable material and have any suitable shape, including, for example, planar, spheroid or particulate. In some embodiments, the support is a scaffolded polymer particle as described in U.S. Published App. No. 20100304982, hereby incorporated by reference in its entirety. In certain embodiments methods comprise depositing at least a portion of an enriched population of library sequences onto a support (e.g., a sequencing support), wherein the support comprises an array of sequencing reaction sites. In some embodiments, an enriched population of library sequences are attached to the sequencing reaction sites on the support. wherein the support comprises an array of 10²-10¹⁰ sequencing reaction sites.

Sequence determination means determination of information relating to the sequence of a nucleic acid and may include identification or determination of partial as well as full sequence information of the nucleic acid. Sequence information may be determined with varying degrees of statistical reliability or confidence. In some embodiments sequence analysis includes high throughput, low depth detection such as by qPCR, rtPCR, and/or array hybridization detection methodologies known in the art. In some embodiments, sequencing analysis includes the determination of the in depth sequence assessment, such as by Sanger sequencing or other high throughput next generation sequencing methods. Next-generation sequencing means sequence determination using methods that determine many (typically thousands to billions) nucleic acid sequences in an intrinsically massively parallel manner, e.g. where many sequences are read out, e.g., in parallel, or alternatively using an ultra-high throughput serial process that itself may be parallelized. Thus, in certain embodiments, methods of the invention include sequencing analysis comprising massively parallel sequencing. Such methods include but are not limited to pyrosequencing (for example, as commercialized by 454 Life Sciences, Inc., Branford, Conn.); sequencing by ligation (for example, as commercialized in the SOLiD™. technology, Life Technologies, Inc., Carlsbad, Calif); sequencing by synthesis using modified nucleotides (such as commercialized in TruSeq™ and HiSeg™. technology by Illumina, Inc., San Diego, Calif; HeliScope™ by Helicos Biosciences Corporation, Cambridge, Mass.; and PacBio Sequel® or RS systems by Pacific Biosciences of California, Inc., Menlo Park, Calif.), sequencing by ion detection technologies (e.g., Ion Torrent™ technology, Life Technologies, Carlsbad, Calif); sequencing of DNA nanoballs (Complete Genomics, Inc., Mountain View, Calif.); nanopore-based sequencing technologies (for example, as developed by Oxford Nanopore Technologies, LTD, Oxford, UK), and like highly parallelized sequencing methods.

For example, in certain embodiments, libraries produced by the teachings of the present disclosure are sufficient in yield to be used in a variety of downstream applications including the Ion Xpress™ Template Kit using an Ion Torrent™ PGM system (e.g., PCR-mediated addition of the nucleic acid fragment library onto Ion Sphere™ Particles)(Life Technologies, Part No. 4467389) or Ion Torrent Proton™ system). For example, instructions to prepare a template library from the amplicon library can be found in the Ion Xpress Template Kit User Guide (Life Technologies, Part No. 4465884), hereby incorporated by reference in its entirety. Instructions for loading the subsequent template library onto the Ion Torrent™ Chip for nucleic acid sequencing are described in the Ion Sequencing User Guide (Part No. 4467391), hereby incorporated by reference in its entirety. Similarly, sequencing using other platforms (e.g., PacBio, Illumina, Helicos, Complete Genomics, Oxford Nanopore) may be carried out using adapted methodologies to incorporate the relevant template preparation according to the instructions and guidance provided with each of the respective platforms.

The initiation point for the sequencing reaction may be provided by annealing a sequencing primer to a product of a solid-phase amplification reaction. In this regard, one or both of the adaptors added during formation of template library may include a nucleotide sequence which permits annealing of a sequencing primer to amplified products derived by whole genome or solid-phase amplification of the template library. Depending on implementation of an embodiment of the invention, a tag sequence and/or target nucleic acid sequence may be determined in a single read from a single sequencing primer, or in multiple reads from two different sequencing primers. In the case of two reads from two sequencing primers, a ‘tag read’ and a ‘target sequence read’ are performed in either order, with a suitable denaturing step to remove an annealed primer after the first sequencing read is completed.

In some embodiments, a sequencer is coupled to server that applies parameters or software to determine the sequence of the amplified target nucleic acid molecules. In certain embodiments, the sequencer is coupled to a server that applies parameters or software to determine the presence of a low frequency mutation allele present in a sample.

### EMBODIMENTS

In one embodiment, a method for preparing a library of target nucleic acid sequences is provided comprising contacting a nucleic acid sample with a plurality of adaptors capable of amplification of one or more target nucleic acid sequences in the sample under conditions wherein the target nucleic acid(s) undergo a first amplification; digesting resulting first amplification products to reduce or eliminate resulting primer dimers and prepare partially digested target amplicons, producing gapped, double stranded amplicons, then repairing the partially digested target amplicons; and amplifying the repaired target amplicons in a second amplification using universal primers, wherein each of the plurality of adaptors comprise a universal handle sequence and a target nucleic acid sequence and a cleavable moiety, wherein at least two and up to one hundred thousand target specific adaptor pairs are included, and wherein the target nucleic acid sequence of the adaptor includes at least one cleavable moiety and the universal handle sequence does not include the cleavable moiety. Optionally one or more tag sequences are comprised in each of the plurality of adaptors. Such methods thereby produce a library of target nucleic acid sequence. In some embodiments, the digestion and repair is carried out in a single step. In particular embodiments the plurality of gapped polynucleotide products in digestion are contacted with the digestion and repair reagents simultaneously. In other embodiments the digestion and repair step is carried out in a temporally separate manner at different temperatures. In particular embodiments the plurality of gapped polynucleotide products in digestion are contacted sequentially with the digestion and repair reagents. In some embodiments one or more of the method steps is conducted in manual mode or in an automated mode or a combination thereof. In particular embodiments each of the method steps is carried out in automated mode. In some embodiments the foregoing methods further comprise at least one purification step. In particular embodiments a purification step is carried out only after the second universal amplification step. In other particular embodiments a purification is carried out after the digestion and repair step and an additional purification is carried out after the second universal amplification. In some of the embodiments adaptor-dimer by products resulting from the first amplification are removed from the resulting library, and in some embodiments an enriched population of amplified target nucleic acids contains a reduced amount of adaptor-dimer byproduct. In certain embodiments, adaptor-dimer byproducts are eliminated. In the foregoing methods the plurality of adaptors capable of amplification of one or more target nucleic acid sequences comprises a multiplex of adaptor pairs capable of amplification of at least two different target nucleic acid sequences. In some embodiments, each target specific pair of the plurality of adaptors includes up to 16,777,216 different adaptor combinations comprising different tag sequences. In certain embodiments each generated target specific amplicon sequence includes at least 1 different sequence and up to 107 different sequences. In some embodiments, the foregoing methods further comprise analyzing the sequence of the resulting library of target nucleic acid sequences. Such analyzing comprises sequencing by traditional sequencing reactions, high throughput next generation sequencing, targeted multiplex array sequence detection, or any combination of two or more of the foregoing. In other embodiments, the foregoing methods further comprise determining the abundance of at least one of the target nucleic acid sequences in the sample. Such determining is carried out by high throughput next generation sequencing in certain embodiments. In particular embodiments, such sequencing is carried out in a bidirectional manner, thereby generating sequence reads in both forward and reverse strands for any given amplicon. In some embodiments the foregoing methods comprise digestion reagent selected from any one or a combination of uracil DNA glycosylase (UDG), apurinic endonuclease (e.g., APE1), RecJf, formamidopyrimidine [fapy]-DNA glycosylase (fpg), Nth endonuclease III, endonuclease VIII, polynucleotide kinase (PNK), Taq DNA polymerase, DNA polymerase I and/or human DNA polymerase beta. In some embodiments, the foregoing methods comprise repair reagent selected from any one or a combination of Phusion DNA polymerase, Phusion U DNA polymerase, SuperFi DNA polymerase, Taq DNA polymerase, Human DNA polymerase beta, T4 DNA polymerase and/or T7 DNA polymerase, SuperFiU DNA polymerase, E. coli DNA ligase, T3 DNA ligase, T4 DNA ligase, T7 DNA ligase, Taq DNA ligase, and/or 9° N DNA ligase. In particular embodiments the foregoing methods comprise digestion and repair reagent selected from any one or a combination of uracil DNA glycosylase (UDG), apurinic endonuclease (e.g., APE1), Taq DNA polymerase, Phusion U DNA polymerase, SuperFiU DNA polymerase, 7 DNA ligase. In more particular embodiments the foregoing methods comprise digestion and repair reagent selected from any one or a combination of uracil DNA glycosylase (UDG), formamidopyrimidine [fapy]-DNA glycosylase (fpg), Phusion U DNA polymerase, Taq DNA polymerase, SuperFiU DNA polymerase, T4 PNK and T7 DNA ligase. In preferred embodiments, the foregoing methods generate compositions comprising nucleic acid library. In particularly preferred embodiments, generated compositions comprising nucleic acid library are useful for analysis of sequences. In specific embodiments, use comprises determination of low frequency allele(s) in a sample.

In one embodiment, a method for preparing a library of target nucleic acid sequences is provided comprising contacting a nucleic acid sample with a plurality of adaptors capable of amplification of one or more target nucleic acid sequences in the sample under conditions wherein the target nucleic acid(s) undergo a first amplification; digesting resulting first amplification products to reduce or eliminate resulting primer dimers and prepare partially digested target amplicons, producing gapped, double stranded amplicons, then repairing the partially digested target amplicons; and amplifying the repaired target amplicons in a second amplification using universal primers, wherein each of the plurality of adaptors comprise a universal handle sequence and a target nucleic acid sequence and a cleavable moiety and a tag sequence is included in at least one adaptor, and the cleavable moieties are included flanking either end of the tag sequence, wherein at least two and up to one hundred thousand target specific adaptor pairs are included, and wherein the target nucleic acid sequence of the adaptor includes at least one cleavable moiety and the universal handle sequence does not include the cleavable moiety. Such methods thereby produce a library of target nucleic acid sequence. In some embodiments, the digestion and repair is carried out in a single step. In particular embodiments the plurality of gapped polynucleotide products in digestion are contacted with the digestion and repair reagents simultaneously. In other embodiments the digestion and repair step is carried out in a temporally separate manner at different temperatures. In particular embodiments the plurality of gapped polynucleotide products in digestion are contacted sequentially with the digestion and repair reagents. In some embodiments one or more of the method steps is conducted in manual mode or in an automated mode or a combination thereof. In particular embodiments each of the method steps is carried out in automated mode. In some embodiments the foregoing methods further comprise at least one purification step. In particular embodiments a purification step is carried out only after the second universal amplification step. In other particular embodiments a purification is carried out after the digestion and repair step and an additional purification is carried out after the second universal amplification. In some of the embodiments adaptor-dimer by products resulting from the first amplification are removed from the resulting library, and in some embodiments an enriched population of amplified target nucleic acids contains a reduced amount of adaptor-dimer byproduct. In certain embodiments, adaptor-dimer byproducts are eliminated. In the foregoing methods the plurality of adaptors capable of amplification of one or more target nucleic acid sequences comprises a multiplex of adaptor pairs capable of amplification of at least two different target nucleic acid sequences. In some embodiments, each target specific pair of the plurality of adaptors includes up to 16,777,216 different adaptor combinations comprising different tag sequences. In certain embodiments each generated target specific amplicon sequence includes at least 1 different sequence and up to 107 different sequences. In some embodiments, the foregoing methods further comprise analyzing the sequence of the resulting library of target nucleic acid sequences. Such analyzing comprises sequencing by traditional sequencing reactions, high throughput next generation sequencing, targeted multiplex array sequence detection, or any combination of two or more of the foregoing. In other embodiments, the foregoing methods further comprise determining the abundance of at least one of the target nucleic acid sequences in the sample. Such determining is carried out by high throughput next generation sequencing in certain embodiments. In particular embodiments, such sequencing is carried out in a bidirectional manner, thereby generating sequence reads in both forward and reverse strands for any given amplicon. In some embodiments the foregoing methods comprise digestion reagent selected from any one or a combination of uracil DNA glycosylase (UDG), apurinic endonuclease (e.g., APE1), RecJf, formamidopyrimidine [fapy]-DNA glycosylase (fpg), Nth endonuclease III, endonuclease VIII, polynucleotide kinase (PNK), Taq DNA polymerase, DNA polymerase I and/or human DNA polymerase beta. In some embodiments, the foregoing methods comprise repair reagent selected from any one or a combination of Phusion DNA polymerase, Phusion U DNA polymerase, SuperFi DNA polymerase, Taq DNA polymerase, Human DNA polymerase beta, T4 DNA polymerase and/or T7 DNA polymerase, SuperFiU DNA polymerase, E. coli DNA ligase, T3 DNA ligase, T4 DNA ligase, T7 DNA ligase, Taq DNA ligase, and/or 9° N DNA ligase. In particular embodiments the foregoing methods comprise digestion and repair reagent selected from any one or a combination of uracil DNA glycosylase (UDG), apurinic endonuclease (e.g., APE1), Taq DNA polymerase, Phusion U DNA polymerase, SuperFiU DNA polymerase, 7 DNA ligase. In more particular embodiments the foregoing methods comprise digestion and repair reagent selected from any one or a combination of uracil DNA glycosylase (UDG), formamidopyrimidine [fapy]-DNA glycosylase (fpg), Phusion U DNA polymerase, Taq DNA polymerase, SuperFiU DNA polymerase, T4 PNK and T7 DNA ligase. In preferred embodiments, the foregoing methods generate compositions comprising nucleic acid library. In particularly preferred embodiments, generated compositions comprising nucleic acid library are useful for analysis of sequences. In specific embodiments, use comprises determination of low frequency allele(s) in a sample.

In one embodiment, a method for preparing a library of target nucleic acid sequences is provided comprising contacting a nucleic acid sample with a plurality of adaptors capable of amplification of one or more target nucleic acid sequences in the sample under conditions wherein the target nucleic acid(s) undergo a first amplification; digesting resulting first amplification products to reduce or eliminate resulting primer dimers and prepare partially digested target amplicons, producing gapped, double stranded amplicons, then repairing the partially digested target amplicons; and amplifying the repaired target amplicons in a second amplification using universal primers, wherein each of the plurality of adaptors comprise a universal handle sequence and a target nucleic acid sequence and a cleavable moiety, wherein at least two and up to one hundred thousand target specific adaptor pairs are included, and wherein the target nucleic acid sequence of the adaptor includes at least one cleavable moiety and the universal handle sequence does not include the cleavable moiety and the melting temperature of each universal sequence is higher than the melting temperature of each target nucleic acid sequence and each tag sequence present. Optionally one or more tag sequences are comprised in each of the plurality of adaptors. Such methods thereby produce a library of target nucleic acid sequence. In some embodiments, the digestion and repair is carried out in a single step. In particular embodiments the plurality of gapped polynucleotide products in digestion are contacted with the digestion and repair reagents simultaneously. In other embodiments the digestion and repair step is carried out in a temporally separate manner at different temperatures. In particular embodiments the plurality of gapped polynucleotide products in digestion are contacted sequentially with the digestion and repair reagents. In some embodiments one or more of the method steps is conducted in manual mode or in an automated mode or a combination thereof. In particular embodiments each of the method steps is carried out in automated mode. In some embodiments the foregoing methods further comprise at least one purification step. In particular embodiments a purification step is carried out only after the second universal amplification step. In other particular embodiments a purification is carried out after the digestion and repair step and an additional purification is carried out after the second universal amplification. In some of the embodiments adaptor-dimer by products resulting from the first amplification are removed from the resulting library, and in some embodiments an enriched population of amplified target nucleic acids contains a reduced amount of adaptor-dimer byproduct. In certain embodiments, adaptor-dimer byproducts are eliminated. In the foregoing methods the plurality of adaptors capable of amplification of one or more target nucleic acid sequences comprises a multiplex of adaptor pairs capable of amplification of at least two different target nucleic acid sequences. In some embodiments, each target specific pair of the plurality of adaptors includes up to 16,777,216 different adaptor combinations comprising different tag sequences. In certain embodiments each generated target specific amplicon sequence includes at least 1 different sequence and up to 107 different sequences. In some embodiments, the foregoing methods further comprise analyzing the sequence of the resulting library of target nucleic acid sequences. Such analyzing comprises sequencing by traditional sequencing reactions, high throughput next generation sequencing, targeted multiplex array sequence detection, or any combination of two or more of the foregoing. In other embodiments, the foregoing methods further comprise determining the abundance of at least one of the target nucleic acid sequences in the sample. Such determining is carried out by high throughput throughput next generation sequencing in certain embodiments. In particular embodiments, such sequencing is carried out in a bidirectional manner, thereby generating sequence reads in both forward and reverse strands for any given amplicon. In some embodiments the foregoing methods comprise digestion reagent selected from any one or a combination of uracil DNA glycosylase (UDG), apurinic endonuclease (e.g., APE1), RecJf, formamidopyrimidine [fapy]-DNA glycosylase (fpg), Nth endonuclease III, endonuclease VIII, polynucleotide kinase (PNK), Taq DNA polymerase, DNA polymerase I and/or human DNA polymerase beta. In some embodiments, the foregoing methods comprise repair reagent selected from any one or a combination of Phusion DNA polymerase, Phusion U DNA polymerase, SuperFi DNA polymerase, Taq DNA polymerase, Human DNA polymerase beta, T4 DNA polymerase and/or T7 DNA polymerase, SuperFiU DNA polymerase, E. coli DNA ligase, T3 DNA ligase, T4 DNA ligase, T7 DNA ligase, Taq DNA ligase, and/or 9° N DNA ligase. In particular embodiments the foregoing methods comprise digestion and repair reagent selected from any one or a combination of uracil DNA glycosylase (UDG), apurinic endonuclease (e.g., APE1), Taq DNA polymerase, Phusion U DNA polymerase, SuperFiU DNA polymerase, 7 DNA ligase. In more particular embodiments the foregoing methods comprise digestion and repair reagent selected from any one or a combination of uracil DNA glycosylase (UDG), formamidopyrimidine [fapy]-DNA glycosylase (fpg), Phusion U DNA polymerase, Taq DNA polymerase, SuperFiU DNA polymerase, T4 PNK and T7 DNA ligase. In preferred embodiments, the foregoing methods generate compositions comprising nucleic acid library. In particularly preferred embodiments, generated compositions comprising nucleic acid library are useful for analysis of sequences. In specific embodiments, use comprises determination of low frequency allele(s) in a sample.

In one embodiment, a method for preparing a library of target nucleic acid sequences is provided comprising contacting a nucleic acid sample with a plurality of adaptors capable of amplification of one or more target nucleic acid sequences in the sample under conditions wherein the target nucleic acid(s) undergo a first amplification; digesting resulting first amplification products to reduce or eliminate resulting primer dimers and prepare partially digested target amplicons, producing gapped, double stranded amplicons, then repairing the partially digested target amplicons; and amplifying the repaired target amplicons in a second amplification using universal primers, wherein each of the plurality of adaptors comprise a universal handle sequence and a target nucleic acid sequence and a cleavable moiety, wherein at least two and up to one hundred thousand target specific adaptor pairs are included, and wherein the target nucleic acid sequence of the adaptor includes at least one cleavable moiety and the universal handle sequence does not include the cleavable moiety. Optionally one or more tag sequences are comprised in each of the plurality of adaptors. Such methods are carried out in a single, addition only workflow reaction, allowing for rapid production of highly multiplexed targeted libraries thereby produce a library of target nucleic acid sequence. In some embodiments, the digestion and repair is carried out in a single step. In particular embodiments the plurality of gapped polynucleotide products in digestion are contacted with the digestion and repair reagents simultaneously. In other embodiments the digestion and repair step is carried out in a temporally separate manner at different temperatures. In particular embodiments the plurality of gapped polynucleotide products in digestion are contacted sequentially with the digestion and repair reagents. In some embodiments one or more of the method steps is conducted in manual mode or in an automated mode or a combination thereof. In particular embodiments each of the method steps is carried out in automated mode. In some embodiments the foregoing methods further comprise at least one purification step. In particular embodiments a purification step is carried out only after the second universal amplification step. In other particular embodiments a purification is carried out after the digestion and repair step and an additional purification is carried out after the second universal amplification. In some of the embodiments adaptor-dimer by products resulting from the first amplification are removed from the resulting library, and in some embodiments an enriched population of amplified target nucleic acids contains a reduced amount of adaptor-dimer byproduct. In certain embodiments, adaptor-dimer byproducts are eliminated. In the foregoing methods the plurality of adaptors capable of amplification of one or more target nucleic acid sequences comprises a multiplex of adaptor pairs capable of amplification of at least two different target nucleic acid sequences. In some embodiments, each target specific pair of the plurality of adaptors includes up to 16,777,216 different adaptor combinations comprising different tag sequences. In certain embodiments each generated target specific amplicon sequence includes at least 1 different sequence and up to 107 different sequences. In some embodiments, the foregoing methods further comprise analyzing the sequence of the resulting library of target nucleic acid sequences. Such analyzing comprises sequencing by traditional sequencing reactions, high throughput next generation sequencing, targeted multiplex array sequence detection, or any combination of two or more of the foregoing. In other embodiments, the foregoing methods further comprise determining the abundance of at least one of the target nucleic acid sequences in the sample. Such determining is carried out by high throughput next generation sequencing in certain embodiments. In particular embodiments, such sequencing is carried out in a bidirectional manner, thereby generating sequence reads in both forward and reverse strands for any given amplicon. In some embodiments the foregoing methods comprise digestion reagent selected from any one or a combination of uracil DNA glycosylase (UDG), apurinic endonuclease (e.g., APE1), RecJf, formamidopyrimidine [fapy]-DNA glycosylase (fpg), Nth endonuclease III, endonuclease VIII, polynucleotide kinase (PNK), Taq DNA polymerase, DNA polymerase I and/or human DNA polymerase beta. In some embodiments, the foregoing methods comprise repair reagent selected from any one or a combination of Phusion DNA polymerase, Phusion U DNA polymerase, SuperFi DNA polymerase, Taq DNA polymerase, Human DNA polymerase beta, T4 DNA polymerase and/or T7 DNA polymerase, SuperFiU DNA polymerase, E. coli DNA ligase, T3 DNA ligase, T4 DNA ligase, T7 DNA ligase, Taq DNA ligase, and/or 9° N DNA ligase. In particular embodiments the foregoing methods comprise digestion and repair reagent selected from any one or a combination of uracil DNA glycosylase (UDG), apurinic endonuclease (e.g., APE1), Taq DNA polymerase, Phusion U DNA polymerase, SuperFiU DNA polymerase, 7 DNA ligase. In more particular embodiments the foregoing methods comprise digestion and repair reagent selected from any one or a combination of uracil DNA glycosylase (UDG), formamidopyrimidine [fapy]-DNA glycosylase (fpg), Phusion U DNA polymerase, Taq DNA polymerase, SuperFiU DNA polymerase, T4 PNK and T7 DNA ligase. In preferred embodiments, the foregoing methods generate compositions comprising nucleic acid library. In particularly preferred embodiments, generated compositions comprising nucleic acid library are useful for analysis of sequences. In specific embodiments, use comprises determination of low frequency allele(s) in a sample.

In one embodiment, a method for preparing a library of target nucleic acid sequences is provided comprising contacting a nucleic acid sample with a plurality of adaptors capable of amplification of one or more target nucleic acid sequences in the sample under conditions wherein the target nucleic acid(s) undergo a first amplification; digesting resulting first amplification products to reduce or eliminate resulting primer dimers and prepare partially digested target amplicons, producing gapped, double stranded amplicons, then repairing the partially digested target amplicons; and amplifying the repaired target amplicons in a second amplification using universal primers, wherein each of the plurality of adaptors comprise a universal handle sequence and a target nucleic acid sequence and a cleavable moiety and all of the adaptors comprise tag sequences having cleavable groups flanking either end of the tag sequence, wherein at least two and up to one hundred thousand target specific adaptor pairs are included, and wherein the target nucleic acid sequence of the adaptor includes at least one cleavable moiety and the universal handle sequence does not include the cleavable moiety. Such methods thereby produce a library of target nucleic acid sequence. In some embodiments, the digestion and repair is carried out in a single step. In particular embodiments the plurality of gapped polynucleotide products in digestion are contacted with the digestion and repair reagents simultaneously. In other embodiments the digestion and repair step is carried out in a temporally separate manner at different temperatures. In particular embodiments the plurality of gapped polynucleotide products in digestion are contacted sequentially with the digestion and repair reagents. In some embodiments one or more of the method steps is conducted in manual mode or in an automated mode or a combination thereof. In particular embodiments each of the method steps is carried out in automated mode. In some embodiments the foregoing methods further comprise at least one purification step. In particular embodiments a purification step is carried out only after the second universal amplification step. In other particular embodiments a purification is carried out after the digestion and repair step and an additional purification is carried out after the second universal amplification. In some of the embodiments adaptor-dimer by products resulting from the first amplification are removed from the resulting library, and in some embodiments an enriched population of amplified target nucleic acids contains a reduced amount of adaptor-dimer byproduct. In certain embodiments, adaptor-dimer byproducts are eliminated. In the foregoing methods the plurality of adaptors capable of amplification of one or more target nucleic acid sequences comprises a multiplex of adaptor pairs capable of amplification of at least two different target nucleic acid sequences. In some embodiments, each target specific pair of the plurality of adaptors includes up to 16,777,216 different adaptor combinations comprising different tag sequences. In certain embodiments each generated target specific amplicon sequence includes at least 1 different sequence and up to 107 different sequences. In some embodiments, the foregoing methods further comprise analyzing the sequence of the resulting library of target nucleic acid sequences. Such analyzing comprises sequencing by traditional sequencing reactions, high throughput next generation sequencing, targeted multiplex array sequence detection, or any combination of two or more of the foregoing. In other embodiments, the foregoing methods further comprise determining the abundance of at least one of the target nucleic acid sequences in the sample. Such determining is carried out by high throughput next generation sequencing in certain embodiments. In particular embodiments, such sequencing is carried out in a bidirectional manner, thereby generating sequence reads in both forward and reverse strands for any given amplicon. In some embodiments the foregoing methods comprise digestion reagent selected from any one or a combination of uracil DNA glycosylase (UDG), apurinic endonuclease (e.g., APE1), RecJf, formamidopyrimidine [fapy]-DNA glycosylase (fpg), Nth endonuclease III, endonuclease VIII, polynucleotide kinase (PNK), Taq DNA polymerase, DNA polymerase I and/or human DNA polymerase beta. In some embodiments, the foregoing methods comprise repair reagent selected from any one or a combination of Phusion DNA polymerase, Phusion U DNA polymerase, SuperFi DNA polymerase, Taq DNA polymerase, Human DNA polymerase beta, T4 DNA polymerase and/or T7 DNA polymerase, SuperFiU DNA polymerase, E. coli DNA ligase, T3 DNA ligase, T4 DNA ligase, T7 DNA ligase, Taq DNA ligase, and/or 9° N DNA ligase. In particular embodiments the foregoing methods comprise digestion and repair reagent selected from any one or a combination of uracil DNA glycosylase (UDG), apurinic endonuclease (e.g., APE1), Taq DNA polymerase, Phusion U DNA polymerase, SuperFiU DNA polymerase, 7 DNA ligase. In more particular embodiments the foregoing methods comprise digestion and repair reagent selected from any one or a combination of uracil DNA glycosylase (UDG), formamidopyrimidine [fapy]-DNA glycosylase (fpg), Phusion U DNA polymerase, Taq DNA polymerase, SuperFiU DNA polymerase, T4 PNK and T7 DNA ligase. In preferred embodiments, the foregoing methods generate compositions comprising nucleic acid library. In particularly preferred embodiments, generated compositions comprising nucleic acid library are useful for analysis of sequences. In specific embodiments, use comprises determination of low frequency allele(s) in a sample.

In one embodiment, provided is a method for preparing a library of target nucleic acid sequences comprising contacting a nucleic acid sample with a plurality of adaptors capable of amplification of one or more target nucleic acid sequences in the sample under conditions wherein the target nucleic acid(s) undergo a first amplification, digesting resulting first amplification products to reduce or eliminate resulting primer dimers and prepare partially digested target amplicons, producing gapped, double stranded amplicons, then repairing the partially digested target amplicons, and amplifying the repaired target amplicons in a second amplification using universal primers; wherein each of the plurality of adaptors comprises a universal handle sequence, one or more tag sequences, a target nucleic acid sequence and a cleavable moiety; and wherein at least two and up to one hundred thousand target specific adaptor pairs are included and wherein the target nucleic acid sequence of the adaptor includes at least one cleavable moiety, cleavable moieties are included in the flanking either end of the tag sequence and the universal handle sequence does not include the cleavable moiety. In certain embodiments the melting temperature of each universal sequence is higher than the melting temperature of each target nucleic acid sequence and each tag sequence present. Such methods thereby produce a library of target nucleic acid sequence. In particular embodiments such methods are carried out in a single, addition only workflow reaction, allowing for rapid production of highly multiplexed targeted libraries. In some embodiments, the digestion and repair is carried out in a single step. In particular embodiments the plurality of gapped polynucleotide products in digestion are contacted with the digestion and repair reagents simultaneously. In other embodiments the digestion and repair step is carried out in a temporally separate manner at different temperatures. In particular embodiments the plurality of gapped polynucleotide products in digestion are contacted sequentially with the digestion and repair reagents. In some embodiments one or more of the method steps is conducted in manual mode or in an automated mode or a combination thereof. In particular embodiments each of the method steps is carried out in automated mode. In some embodiments the foregoing methods further comprise at least one purification step. In particular embodiments a purification step is carried out only after the second universal amplification step. In other particular embodiments a purification is carried out after the digestion and repair step and an additional purification is carried out after the second universal amplification. In some of the embodiments adaptor-dimer by products resulting from the first amplification are removed from the resulting library, and in some embodiments an enriched population of amplified target nucleic acids contains a reduced amount of adaptor-dimer byproduct. In certain embodiments, adaptor-dimer byproducts are eliminated. In the foregoing methods the plurality of adaptors capable of amplification of two or more target nucleic acid sequences comprises a multiplex of adaptor pairs capable of amplification of target nucleic acid sequences. In certain embodiments all of the adaptors comprise tag sequences having cleavable groups flanking either end of the tag sequences. In some embodiments, each target specific pair of the plurality of adaptors includes up to 16,777,216 different adaptor combinations comprising different tag sequences. In certain embodiments each generated target specific amplicon sequence includes at least 1 different sequence and up to 107 different sequences. In some embodiments, the foregoing methods further comprise analyzing the sequence of the resulting library of target nucleic acid sequences. Such analyzing comprises sequencing by traditional sequencing reactions, high throughput next generation sequencing, targeted multiplex array sequence detection, or any combination of two or more of the foregoing. In other embodiments, the foregoing methods further comprise determining the abundance of at least one of the target nucleic acid sequences in the sample. Such determining is carried out by high throughput next generation sequencing in certain embodiments. In particular embodiments, such sequencing is carried out in a bidirectional manner, thereby generating sequence reads in both forward and reverse strands for any given amplicon. In some embodiments the foregoing methods comprise digestion reagent selected from any one or a combination of uracil DNA glycosylase (UDG), apurinic endonuclease (e.g., APE1), RecJf, formamidopyrimidine [fapy]-DNA glycosylase (fpg), Nth endonuclease III, endonuclease VIII, polynucleotide kinase (PNK), Taq DNA polymerase, DNA polymerase I and/or human DNA polymerase beta. In some embodiments, the foregoing methods comprise repair reagent selected from any one or a combination of Phusion DNA polymerase, Phusion U DNA polymerase, SuperFi DNA polymerase, Taq DNA polymerase, Human DNA polymerase beta, T4 DNA polymerase and/or T7 DNA polymerase, SuperFiU DNA polymerase, E. coli DNA ligase, T3 DNA ligase, T4 DNA ligase, T7 DNA ligase, Taq DNA ligase, and/or 9° N DNA ligase. In particular embodiments the foregoing methods comprise digestion and repair reagent selected from any one or a combination of uracil DNA glycosylase (UDG), apurinic endonuclease (e.g., APE1), Taq DNA polymerase, Phusion U DNA polymerase, SuperFiU DNA polymerase, 7 DNA ligase. In more particular embodiments the foregoing methods comprise digestion and repair reagent selected from any one or a combination of uracil DNA glycosylase (UDG), formamidopyrimidine [fapy]-DNA glycosylase (fpg), Phusion U DNA polymerase, Taq DNA polymerase, SuperFiU DNA polymerase, T4 PNK and T7 DNA ligase. In preferred embodiments, the foregoing methods generate compositions comprising nucleic acid library. In particularly preferred embodiments, generated compositions comprising nucleic acid library are useful for analysis of sequences. In specific embodiments, use comprises determination of low frequency allele(s) in a sample.

In one embodiment provided is a composition comprising a plurality of nucleic acid adaptors, wherein each of the plurality of adaptors comprise a 5′ universal handle sequence, one or more tag sequences, and a 3′ target nucleic acid sequence wherein each adaptor comprises a cleavable moiety, the target nucleic acid sequence of the adaptor includes at least one cleavable moiety, cleavable moieties are included flanking either end of the tag sequence and the universal handle sequence does not include the cleavable moiety, and at least two and up to one hundred thousand target specific adaptor pairs are included. In some embodiments the melting temperature of each adaptor universal sequence is higher than the melting temperature of each target nucleic acid sequence and each tag sequence present in the same adaptor. The provided compositions allow for rapid production of highly multiplexed targeted libraries. In particular embodiments, the composition comprises multiplex of adaptor pairs capable of amplification of at least two different target nucleic acid sequences. In certain embodiments, each target specific pair of the plurality of adaptors includes up to 16,777,216 different adaptor combinations comprising different tag sequences In certain embodiments, compositions each generated target specific amplicon produced by target specific pairs of the plurality of adaptors produces at least 1 different sequence and up to 107 different sequences. The foregoing compositions comprise adaptors wherein they are single stranded or double stranded. Yet additional embodiments provide kits comprising the adaptor compositions of any of the foregoing embodiments. In some embodiments such kits further comprise any one or more of an amplification reagent, a digestion reagent and a repair reagent. In certain embodiments such kits further comprise an amplification reagent, a digestion reagent and a repair reagent.

### EXEMPLIFICATION

### Example 1

Provided methods of the invention comprise streamlined procedures enabling rapid, highly multiplexed PCR. See FIG. 1. The invention optionally allows for the incorporation of one or more unique tag sequences, if so desired. Exemplary methods of the invention comprise the following protocols:

### Example 1A

### Materials and Method

Optional Reverse Transcription (RT) Reaction method (10 uL reaction)_may be carried out in samples where RNA and DNA are analyzed:

### Materials

2 uL 5× SuperScript™ VILO™ (Thermo Fisher Scientific) mix into a microtube or microwell, ≤8 uL volume of DNA+RNA sample for ≤20 ng total amount of DNA+RNA sample (˜1% RNA sample of the total nucleic acid (TNA));
nuclease-free H2O to the above tube/well to make 10 uL total reaction volume;

### Method:

42 C for 30 min
85 C for 1 min
4 C hold (indefinitely)

### Amplification:

### Materials

ul dH₂O (to 30 ul final)
ul 20 ng genomic DNA sample
48 nM Panel of Adaptors
15 ul PhusionU multiplex PCR master mix
2.4 ul 2 u/ul Phusion U DNA polymerase

### Amplification:

98 C for 2 min
3 cycles of the following:
   98 C for 30 s
   64 C for 2 min
   62 C for 2 min
   60 C for 4 min
   58 C for 2 min
   72 C for 30 s
72 C for 2 min
4 C hold (indefinitely).

### Digestion, Fill-in, Ligation:

### Materials

2 ul (5 u/ul) UDG,
4 ul (10 u/ul) FPG
0.5 ul (10 u/ul) T4 PNK
1 ul (3000 u/ul) T7 ligase
1 ul (10 mM) ATP.

### Method

Mix the materials above, add to reaction mixture.
Incubate:
   30 C for 20 min
   55 C for 20 min
   25 C for 10 min
   98 C for 2 min
   4 C hold (indefinitely)

The resulting repaired sample is purified using 35 ul Ampure® beads (Beckman Coulter, Inc.) according to the manufacturer instructions.

### Amplification:

### Materials

1 ul for each P1 and A-universal primers, optionally containing barcode sequence (Ion Xpress™ Adapters, Thermo Fisher Scientific)

### Method

Incubate:
   98 C for 2 min
22 cycles of
   98 C for 15 s
   64 C for 15 s
   72 C for 15 s
72 C for 5 min
4 C hold (indefinitely)

The resulting sample is purified using 35 ul Ampure® beads (Beckman Coulter, Inc.) according to the manufacturer instructions. Optionally, the purification step is repeated 1× to 2×.

### Example 1B

### Materials and Method

Optional Reverse Transcription (RT) Reaction method (10 uL reaction)_may be carried out in samples where RNA and DNA are analyzed:

### Materials

2 uL 5× SuperScript™ VILO™ (Thermo Fisher Scientific) mix into a microtube or microwell, ≤8 uL volume of DNA+RNA sample for ≤20 ng total amount of DNA+RNA sample (˜1% RNA sample of the total nucleic acid (TNA));
nuclease-free H2O to the above tube/well to make 10 uL total reaction volume;

### Method:

42 C for 30 min
85 C for 1 min
4 C hold (indefinitely)

### Amplification:

### Materials

ul dH₂O (to 30 ul final)
ul 20 ng genomic DNA sample
48 nM Panel of Adaptors
15 ul PhusionU multiplex PCR master mix
2.4 ul 2 u/ul Phusion U DNA polymerase

### Amplification:

98 C for 2 min
3 cycles of the following:
   98 C for 30 s
   64 C for 2 min
   62 C for 2 min
   60 C for 4 min
   58 C for 2 min
   72 C for 30 s
72 C for 2 min
4 C hold (indefinitely).

### Digestion, Fill-in, Ligation:

### Materials

2 ul (5 u/ul) UDG,
4 ul (10 u/ul) APE1
0.5 ul (1 u/ul) Taq polymerase
1 ul (3000 u/ul) T7 ligase
1 ul (10 mM) ATP.

### Method

Mix the materials above, add to reaction mixture.
Incubate:
   30 C for 20 min
   55 C for 20 min
   25 C for 10 min
   98 C for 2 min
   4 C hold (indefinitely)

### Amplification:

### Materials

1 ul for each P1 and A-universal primers, optionally containing barcode sequence (Ion Xpress™ Adapters, Thermo Fisher Scientific)

### Method

Incubate:
   98 C for 2 min
22 cycles of
   98 C for 15 s
   64 C for 15 s
   72 C for 15 s
72 C for 5 min
4 C hold (indefinitely)

The resulting sample is purified using 35 ul Ampure® beads (Beckman Coulter, Inc.) according to the manufacturer instructions. Optionally, purification step may be repeated 1× to 2×.

### Example 1C

### Materials and Method

Optional Reverse Transcription (RT) Reaction method (10 uL reaction) may be carried out in samples where RNA and DNA are analyzed:

### Materials

2 uL 5× SuperScript™ VILO™ (Thermo Fisher Scientific) mix into a microtube or microwell, ≤8 uL volume of DNA+RNA sample for ≤20 ng total amount of DNA+RNA sample (˜1% RNA sample of the total nucleic acid (TNA));
nuclease-free H2O to the above tube/well to make 10 uL total reaction volume;

### Method:

42 C for 30 min
85 C for 1 min
4 C hold (indefinitely)

### Amplification:

### Materials

_ ul dH₂O (to 30 ul final)
_ ul Genomic DNA sample (˜20 ng)
6 ul Adaptor Panel 250 nM
15 ul PhusionU multiplex PCR master mix (F-562)
3.0 ul 2 u/ul SuperFiU DNA Polymerase

### Amplification

Assemble mixture of materials in reaction in 96-well plate wells, amplify using method:
99 C for 2 min
3 cycles of the following:
   99 C for 30 s
   64 C for 2 min
   62 C for 2 min
   60 C for 4 min
   58 C for 2 min
   72 C for 30 s
72 C for 2 min
4 C hold (indefinitely)

### Digestion, Fill-in, Ligation:

### Materials

0.1 ul VIP Oligo 10 uM (P/N 4385451 Thermo Fisher Scientific, Inc.)
2 ul (5 u/ul) UDG
4 ul (10 u/ul) APE1 (NEB, M0282L)
0.5 ul (1 u/ul) Taq polymerase (EP0404)
1 ul (3000 u/ul) T7 ligase (NEB M0318L)
1 ul (10 mM) ATP

### Method

Mix the above materials, add into reaction mixture
Incubate:
30 C for 15 min
50 C for 15 min
55 C for 15 min
25 C for 10 min
98 C for 2 min
4 C hold (indefinitely)

### Amplification

### Materials

1 ul for each P1 and A-Barcode-universal primers optionally containing barcode sequence (Ion Xpress™ Adapters, Thermo Fisher Scientific)

### Method

Add into the reaction wells the above materials, amplify:
99 C for 2 min
20 cycles:
   99 C for 20 s
   64 C for 20 s
   72 C for 20 s
72 C for 5 min
4 C hold (indefinitely)

The resulting sample is purified using 1× Ampure® beads (Beckman Coulter, Inc.) according to the manufacturer instructions. Optionally, purification step may be repeated 1× to 2×.

### Example 1D

### Materials and Method

Optional Reverse Transcription (RT) Reaction method (10 uL reaction) may be carried out in samples where RNA and DNA are analyzed:

### Materials

2 uL 5× SuperScript™ VILO™ (Thermo Fisher Scientific) mix into a microtube or microwell, ≤8 uL volume of DNA+RNA sample for ≤20 ng total amount of DNA+RNA sample (˜1% RNA sample of the total nucleic acid (TNA));
nuclease-free H2O to the above tube/well to make 10 uL total reaction volume;

### Method:

42 C for 30 min
85 C for 1 min
4 C hold (indefinitely)

### Amplification:

### Materials

_x_ul nuclease free dH₂O (x to 30 ul final)
_y_ul Genomic DNA sample (y ˜20 ng) or y 10 uL of RT reaction for DNA+RNA sample
12.5 ul Adaptor Panel for ˜50 nM each primer concentration
7.5 ul Platinum™ SuperFi™ PCR master mix, replacing SuperFi enzyme with 0.96 U/μL SuperFiU™ DNA Polymerase
3.0 ul 2 U/ul SuperFiU™ DNA Polymerase
optionally, an control may be included in reaction, (e.g, Acrometrix Oncology Hotspot Control (Thermo Fisher Scientific))

### Amplification

Assemble mixture of materials in reaction in 96-well plate wells, seal, vortex and centrifuge plate, amplify using method:
99 C for 1 s
3 cycles of the following:
99 C for 30 s
64 C for 2 min
60 C for 6 min
72 C for 30 s
then 72 C for 2 min
4 C hold (indefinitely)

### Digestion, Fill-in, Ligation:

### Materials

0.1 ul VIP Oligo 0.2 uM (P/N 4385451 Thermo Fisher Scientific, Inc.)
2 ul (5 u/ul) UDG
4 ul (8 U/ul) APE1 (NEB, M0282L)
0.5 ul (0.1 U/ul) Taq polymerase (EP0404)
1 ul (6000 U/ul) T7 ligase (NEB M0318L)
1 ul (2 mM) ATP
0.5 ul mAB2A7 (0.6 mg/mL)
0.25 ul mAB5D3 (0.25 mg/mL)

### Method

Mix the above materials, add into reaction mixture, seal plate, vortex and centrifuge
Incubate:
30 C for 15 min
50 C for 15 min
55 C for 15 min
25 C for 10 min
98 C for 2 min
4 C hold (indefinitely)

### Amplification

### Materials

1 ul for each P1 and A-Barcode-universal primers optionally barcoded sequence (Ion Xpress™ Adapters, Thermo Fisher Scientific); or 1 uL each of 10 uM BC1-Ah, and 1 uL of 10 uM P1-Plh (IonCode™ Barcode Adapters, Thermo Fisher Scientific), for uni-directional library

### Method

Add into the reaction wells the above materials, seal plate, vortex and centrifuge, then amplify:
99 C for 15 s
5 cycles:
   99 C for 15 s
   62 C for 20 s
   72 C for 20 s
15 cycles:
   99 C for 15 s
   70 C for 40 s
72 C for 5 min
4 C hold (indefinitely)

The resulting sample is purified using 1× Ampure® beads (Beckman Coulter, Inc.) according to the manufacturer instructions.

Optionally, purification may be repeated 1× to 2×.

### Example 1E

### Materials and Method

### Amplification:

### Materials

x__ul dH₂O (x to 30 ul final)
y__ul Genomic DNA sample (y˜20 ng) or y 10 uL of RT reaction for DNA+RNA sample
12.5 ul Adaptor Panel for ˜50 nM each primer concentration
7.5 ul Platinum™ SuperFi™ PCR master mix, replacing SuperFi enzyme with 0.96 U/μL SuperFiU™ DNA Polymerase
3.0 ul 2 u/ul SuperFiU™ DNA Polymerase

### Amplification

Assemble mixture of materials in reaction in 96-well plate wells, seal plate, vortex and centrifuge, then amplify using method:
99 C for 1 s
3 cycles of the following:
99 C for 30 s
64 C for 2 min
60 C for 6 min
72 C for 30 s
72 C for 2 min
4 C hold (indefinitely)

### Digestion, Fill-in, Ligation:

### Materials

0.1 ul VIP Oligo 0.2 uM (P/N 4385451 Thermo Fisher Scientific, Inc.)
2 ul (5 u/ul) UDG
4 ul (8 U/ul) APE1 (NEB, M0282L)
0.5 ul (0.1 U/ul) Taq polymerase (EP0404)
1 ul (6000 U/ul) T7 ligase (NEB M0318L)
1 ul (2 mM) ATP
0.5 ul mAB2A7 (0.6 mg/mL)
0.25 ul mAB5D3 (0.25 mg/mL)

### Method

Mix the above materials, add into reaction mixture, seal plate, vortex and centrifuge
Incubate:
30 C for 15 min
50 C for 15 min
55 C for 15 min
25 C for 10 min
98 C for 2 min
4 C hold (indefinitely)

### Amplification

### Materials

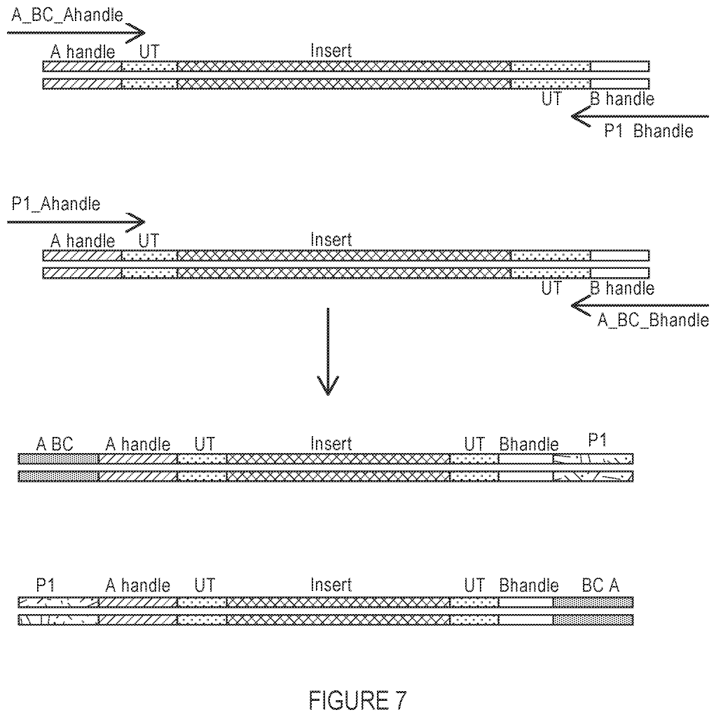

1 uL of 10 uM BC1-Ah, 1 uL of 10 uM P1-Uh, 1.5 uL of 10 uM BC1-Uh, and 1.5 uL of 10 uM P1-Ah. herein for bi-directional library preparation. BC1-Ah comprises barcode sequence and complementary sequence to universal A handle of forward adapters herein; BC1-Uh comprises barcode sequence and complementary sequence to universal handle of any of reverse adapters B, C, D, or E herein; P1-Uh comprises Ion adapter P1 adapter sequence, barcode sequence, and complementary sequence to universal B, C, D, or E handle of any of reverse adapters B, C, D, or E herein; P1-Ah comprises Ion adapter P1 adapter sequence, barcode sequence, and complementary sequence to universal handle of A handle of forward adapters herein. See FIG. 7.

### Method

Add into the reaction wells the above materials, seal plate, vortex, centrifuge then amplify:
99 C for 15 s
5 cycles:
99 C for 15 s
62 C for 20 s
72 C for 20 s
15 cycles:
99 C for 15 s
70 C for 40 s
72 C for 5 min
4 C hold (indefinitely)

The resulting sample is purified using 1× Ampure® beads (Beckman Coulter, Inc.) according to the manufacturer instructions.

Optionally, purification may be repeated 1× to 2×.

### Example 1F

### Materials and Method

Optional Reverse Transcription (RT) Reaction method (10 uL reaction)_may be carried out in samples where RNA and DNA are analyzed:

### Materials

2 uL 5× SuperScript™ VILO™ (Thermo Fisher Scientific) mix into a microtube or microwell, ≤8 uL volume of DNA+RNA sample for ≤20 ng total amount of DNA+RNA sample (˜1% RNA sample of the total nucleic acid (TNA));
nuclease-free H2O to the above tube/well to make 10 uL total reaction volume;

### Method:

42 C for 30 min
85 C for 1 min
4 C hold (indefinitely)

### Amplification:

### Materials

_x_ul nuclease free dH₂O (x to 30 ul final)
_y_ul Genomic DNA sample (y ˜20 ng) or y 10 uL of RT reaction for DNA+RNA sample
12.5 ul Adaptor Panel for ˜50 nM each primer concentration
7.5 ul Platinum™ SuperFi™ PCR master mix, replacing SuperFi enzyme with 0.96 U/μL SuperFiU™ DNA Polymerase
3.0 ul 2 U/ul SuperFiU™ DNA Polymerase
optionally, a control may be included in reaction, (e.g, Acrometrix Oncology Hotspot Control (Thermo Fisher Scientific))

### Amplification

Assemble mixture of materials in reaction in 96-well plate wells, seal, vortex and centrifuge plate, amplify using method:
99 C for 1 s
3 cycles of the following:
99 C for 30 s
64 C for 2 min
60 C for 6 min
72 C for 30 s
then 72 C for 2 min
4 C hold (indefinitely)

### Digestion, Fill-in, Ligation:

### Materials

0.1 ul VIP Oligo 0.2 uM (P/N 4385451 Thermo Fisher Scientific, Inc.)
2 ul (5 u/ul) UDG
4 ul (8 U/ul) APE1 (NEB, M0282L)
0.5 ul (0.1 U/ul) Taq polymerase (EP0404)
1 ul (6000 U/ul) T7 ligase (NEB M0318L)
1 ul (2 mM) ATP
0.5 ul mAB2A7 (0.6 mg/mL)
0.25 ul mAB5D3 (0.25 mg/mL)

### Method

Mix the above materials, add into reaction mixture, seal plate, vortex and centrifuge
Incubate:
30 C for 15 min
50 C for 15 min
55 C for 15 min
25 C for 10 min
98 C for 2 min
4 C hold (indefinitely)

### Amplification

### Materials

1 ul for each of (1) P5-index-A-handle primer; (2) P5-index-I-handle primer; (3) P7-index-A-handle primer; and (4) P7-index-I-handle primer. See Table F.

### Method

Add into the reaction wells the above materials, seal plate, vortex and centrifuge, then amplify:
99 C for 15 s
5 cycles:
   99 C for 15 s
   62 C for 20 s
   72 C for 20 s
15 cycles:
   99 C for 15 s
   70 C for 40 s
72 C for 5 min
4 C hold (indefinitely)

The resulting sample is purified using 1× Ampure® beads (Beckman Coulter, Inc.) according to the manufacturer instructions.

Optionally, purification may be repeated 1× to 2×.

### Example 2

The first step of provided methods comprises a few rounds of amplification, for example, three to six cycles of amplification, and in certain instances, three cycles of amplification using forward and reverse adaptors to each gene specific target sequence. Each adaptor contains a 5′ universal sequence, and a 3′ gene specific target sequence. In some embodiments adaptors optionally comprise a unique tag sequence located between the 5′ universal and the 3′ gene specific target sequences.

In specific embodiments wherein unique tag sequences are utilized, each gene specific target adaptor pair includes a multitude of different unique tag sequences in each adaptor. For example, each gene specific target adaptor comprises up to 4096 TAGs. Thus, each target specific adaptor pair comprises at least four and up to 16,777,216 possible combinations.

Each of the provided adaptors comprises a cleavable uracil in place of thymine at specific locations in the forward and reverse adaptor sequences. Positions of uracils (Us) are consistent for all forward and reverse adaptors having unique tag sequences, wherein uracils (Us) are present flanking the 5′ and 3′ ends of the unique tag sequence when present; and Us are present in each of the gene specific target sequence regions, though locations for each gene specific target sequence will inevitably vary. Uracils flanking each unique tag sequence (UT) and in gene-specific sequence regions are designed in conjunction with sequences and calculated Tm of such sequences, to promote fragment dissociation at a temperature lower than melting temperature of the universal handle sequences, which are designed to remain hybridized at a selected temperature. Variations in Us in the flanking sequences of the UT region are possible, however designs keep the melting temperature below that of the universal handle sequences on each of the forward and reverse adaptors. Exemplary adaptor sequence structures comprise:

Forward Adaptor:
SEQ ID NO: 1
------A Handle----- ------*UT*------ --Gene Specific--
TCTGTACGGTGACAAGGCG-U-NNNACTNNNTGA-U-GAGGACCGUCGCTUGGT
Rev Adaptor B
SEQ ID NO: 2
CTCTATGGGCAGTCGGTGAT-U-NNNACTNNNTGA-U-CCTTCTGCAUGGTATTCTTTCTCTUCC
-----B Handle------- --------*UT*---- -------Gene Specific-------
Rev Adaptor C
SEQ ID NO: 3
TCTAGTCGGTCAGTCACGG-U-NNNACTNNNTGA-U-CCTTCTGCAUGGTATTCTTTCTCTUCC
-----C Handle------- ------UT------- -------Gene Specific-------
Rev Adaptor D
SEQ ID NO: 4
TCTAGTGCTGCAGTCACGG-U-NNNACTNNNTGA-U-CCTTCTGCAUGGTATTCTTTCTCTUCC
-----D Handle------- ------UT------- -------Gene Specific--------
Rev Adaptor E
SEQ ID NO: 5
TGACAAGGCGTAGTCACGG-U-NNNACTNNNTGA-U-CCTTCTGCAUGGTATTCTTTCTCTUCC
-----E Handle------- ------UT------- -------Gene Specific-------

Wherein each N is a base selected from A, C, G, or T and the constant sections of the UT region are used as anchor sequences to ensure correct identification of variable (N) portion. The constant and variable regions of the UT can be significantly modified (e.g., alternative constant sequence, >3 Ns per section) as long as the Tm of the UT region remains below that of the universal handle regions. Importantly, cleavable uracils are absent from each forward (e.g., TCTGTACGGTGACAAGGCG (SEQ ID NO:6) and reverse (e.g., CTCTATGGGCAGTCGGTGAT (SEQ ID NO:7) universal handle sequence.

Enzymes used for amplification include (but are not limited to): Phusion U DNA polymerase; SuperFi U DNA polymerase; Taq DNA polymerase; Veraseq Ultra DNA polymerase. SuperFi U DNA Polymerase is a modified version of high fidelity SuperFi DNA Polymerase, available from Thermo Fisher Scientific. SuperFiU DNA comprises a modification in the uracil-binding pocket (e.g., AA 36) and a family B polymerase catalytic domain (e.g., AA 762). SuperFiU is described in U.S. Provisional patent application No. 62/524,730 filed Jun. 26, 2017, and International Patent application no. PCT/EP2018/066896, filed Jun. 25, 2018 which are each hereby incorporated by reference. Polymerase enzymes may be limited in their ability to utilize uracil and/or any alternative cleavable residues (e.g., inosine, etc.) included into adaptor sequences. In certain embodiments, it may also be advantageous to use a mixture of polymerases to reduce enzyme specific PCR errors.

The second step of methods involves partial digestion of resulting amplicons, as well as any unused uracil-containing adaptors. For example, where uracil is incorporated as a cleavable site, digestion and repair includes enzymatic cleavage of the uridine monophosphate from resulting primers, primer dimers and amplicons, and melting DNA fragments, then repairing gapped amplicons by polymerase fill-in and ligation. This step reduces and potentially eliminates primer-dimer products that occur in multiplex PCR. In some instances, digestion and repair are carried out in a single step. In certain instances, it may be desirable to separate digestion and repair-steps temporally. For example, thermolabile polymerase inhibitors may be utilized in conjunction with methods, such that digestion occurs at lower temperatures (25-40° C.), then repair is activated by increasing temperature enough to disrupt a polymerase-inhibitor interaction (e.g., polymerase-Ab), though not high enough to melt the universal handle sequences.

Uracil-DNA Glycosylase (UDG) enzyme can be used to remove uracils, leaving abasic sites which can be acted upon by several enzymes or enzyme combinations including (but not limited to): APE 1-Apurinic/apyrimidinic endonuclease; FPG-Formamidopyrimidine [fapy]-DNA glycosylase; Nth-Endonuclease III; Endo VIII-Endonuclease VIII; PNK-Polynucleotide Kinase; Taq-Thermus aquaticus DNA polymerase; DNA pol I-DNA polymerase I; Pol beta-Human DNA polymerase beta. In a particular implementation, the method uses Human apurinic/apyrimidinic endonuclease, APE1. APE1 activity leaves a 3′-OH and a 5′ deoxyribose-phosphate (5′-dRP). Removal of the 5′-dRP can be accomplished by a number of enzymes including recJ, Polymerase beta, Taq, DNA pol I, or any DNA polymerase with 5′-3′ exonuclease activity. Removal of the 5′-dRP by any of these enzymes creates a ligatable 5′-phosphate end. In another implementations, UDG activity removes the Uracil and leaves and abasic site which is removed by FPG, leaving a 3′ and 5′-phosphate. The 3′-phosphate is then removed by T4 PNK, leaving a polymerase extendable 3′-OH. The 5′-deoxyribose phosphate can then be removed by Polymerase beta, fpg, Nth, Endo VIII, Taq, DNA pol I, or any other DNA polymerase with 5′-3′ exonuclease activity. In a particular implementation Taq DNA polymerase is utilized.

Repair fill-in process can be accomplished by almost any polymerase, possibly the amplification polymerase used for amplification in step 1 or by any polymerase added in step 2 including (but not limited to): Phusion DNA polymerase; Phusion U DNA polymerase; SuperFi DNA polymerase; SuperFi U DNA polymerase; TAQ; Pol beta; T4 DNA polymerase; and T7 DNA polymerase. Ligation repair of amplicons can be performed by many ligases including (but not limited to): T4 DNA ligase; T7 DNA ligase; Taq DNA ligase. In a particular implementation of the methods, Taq DNA polymerase is utilized and ligation repaired in accomplished by T7 DNA ligase.

A last step of library preparation involves amplification of the repaired amplicons by standard PCR protocols using universal primers that contain sequences complementary to the universal handle sequences on the 5′ and 3′ ends of prepared amplicons. For example, an A-universal primer, and a P1 universal primer, each part of the Ion Express Adaptor Kit (Thermo Fisher Scientific, Inc.) may optionally contain a sample specific barcode. The last library amplification step may be performed by many polymerases including, but not limited to: Phusion DNA polymerase; Phusion U DNA polymerase; SuperFi DNA polymerase; SuperFi U DNA polymerase; Taq DNA polymerase; Veraseq Ultra DNA polymerase.

2A, In one specific implementation, adaptors were designed using the composition design approach provided herein, including universal handle-unique tag-gene specific target sequence described in Example 2 above, and targeted to genes using the ONCOMINE™ Focus Research Panel (Thermo Fisher Scientific, Inc.) target sequences and ION AMPLISEQ™ Designer (Thermo Fisher Scientific, Inc). Forward and reverse adaptors described above were utilized comprising

Forward Adaptor:
SEQ ID NO: 1
------A Handle----- ------*UT*------ --Gene Specific--
TCTGTACGGTGACAAGGCG-U-NNNACTNNNTGA-U-GAGGACCGUCGCTUGGT
Rev Adaptor B
SEQ ID NO: 2
CTCTATGGGCAGTCGGTGAT-U-NNNACTNNNTGA-U-CCTTCTGCAUGGTATTCTTTCTCTUCC
-----B Handle------- --------*UT*---- -------Gene Specific-------

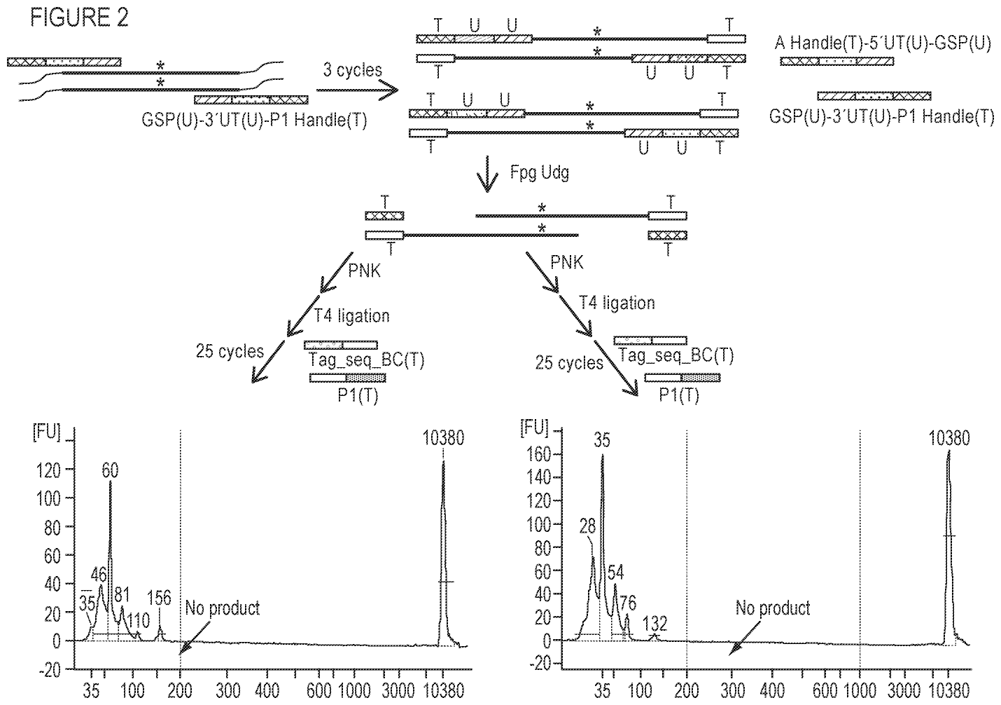

With target sequences specific to targets as in Table A, and adaptors each comprise 4096 unique tag sequences for each gene specific target sequence, resulting in an estimate of 16,777,216 different unique tag combinations for each gene specific target sequence pair. Preparation of library was carried out according to the method described above for Example 1A. Formamidopyrimidine [fapy]-DNA glycosylase (FPG)/UDG enzyme is utilized for digestion, which is expected to create abasic sites at all uracil positions, FPG is expected to cleave on the 5′ and 3′ side of the abasic site (leaving a 3′-phosphate and a 5′ phosphate) and removal of the 3′ phosphate (by T4 PNK for example) should produce an extendable 3′-OH and a ligatable 5′-phosphate. However, as shown by the BioAnalyzer trace (See FIG. 2), this process consistently failed to generate recoverable product. The process can be rescued however by the addition of an additional purification step post-repair. The purification process can be anything inactivates and removes the repair enzymes prior to the next amplification step. Similar results were obtained if endoVIII was utilized.

2B. In another specific implementation, adaptors were prepared as described in section 2A for targets of the ONCOMINE™ Focus Assay. See Table B. Forward and reverse adaptors described above were utilized comprising

Forward Adaptor:
SEQ ID NO: 1
------A Handle----- ------*UT------ --Gene Specific--
TCTGTACGGTGACAAGGCG-U-NNNACTNNNTGA-U-GAGGACCGUCGCTUGGT
Reverse Adaptor was any of Rev Adaptor B, Rev Adaptor C, Rev
Adaptor D, Rev Adaptor E: Rev Adaptor B
SEQ ID NO: 2
CTCTATGGGCAGTCGGTGAT-U-NNNACTNNNTGA-U-CCTTCTGCAUGGTATTCTTTCTCTUCC
-----B Handle------- --------*UT*---- -------Gene Specific-------
Rev Adaptor C
SEQ ID NO: 3
TCTAGTCGGTCAGTCACGG-U-NNNACTNNNTGA-U-CCTTCTGCAUGGTATTCTTTCTCTUCC
-----C Handle------- ------UT------- -------Gene Specific-------
Rev Adaptor D
SEQ ID NO: 4
TCTAGTGCTGCAGTCACGG-U-NNNACTNNNTGA-U-CCTTCTGCAUGGTATTCTTTCTCTUCC
-----D Handle------- ------UT------- -------Gene Specific-------
Rev Adaptor E
SEQ ID NO: 5
TGACAAGGCGTAGTCACGG-U-NNNACTNNNTGA-U-CCTTCTGCAUGGTATTCTTTCTCTUCC
-----E Handle------- ------UT------- -------Gene Specific-------

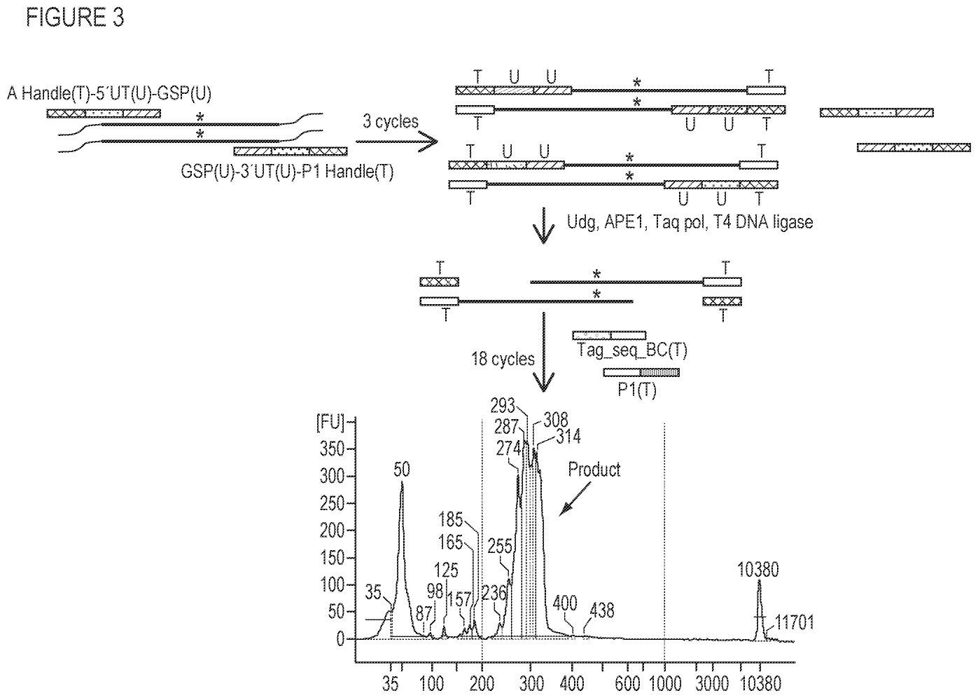

With target sequences specific to targets as in Table B, and adaptors each comprise 4096 unique tag sequences for each gene specific target sequence, resulting in an estimate of 16,777,216 different unique tag combinations for each gene specific target sequence pair. Preparation of library was carried out according to the method described above for 1C. See FIG. 3, Table 1. Similar successful sequencing results were generated with each of the reverse adaptor pairings.

### Example 3

Prepared libraries are sequenced, and analyzed. Sequencing can be carried out by a variety of known methods, including, but not limited to sequencing by synthesis, sequencing by ligation, and/or sequencing by hybridization. Sequencing has been carried out in the examples herein using the Ion Torrent platform (Thermo Fisher Scientific, Inc.), however, libraries can be prepared and adapted for analysis, e.g., sequencing, using any other platforms, e.g., Illumina, PacBio, etc. Result may be analyzed using a number of metrics to assess performance, for example:
# of families (with ng input DNA captured) The median # of families is a measure of the number of families that maps to an individual target. In this case, each unique molecular tag is a family.
Uniformity is a measure of the percentage of target bases covered by at least 0.2× the average read depth. This metric is used to ensure that the technology does not selectively under-amplify certain targets.
Positives/Negatives: When a control sample with known mutations is utilized is analyzed (e.g., Acrometrix Oncology Hotspot Control DNA, Thermo Fisher Scientific, Inc.), the number of True Positives can be tracked.
   True Positives: The number of True Positives informs on the number of mutations that were present and correctly identified.
   False positives (FP): (Hot spot and Whole Target) The number of False Positives informs on the number of mutations that are determined to be present, but known not to be in the sample.
   False negatives (FN) (if acrometrix spike-in is used) The number of False Negatives informs on the number of mutations that were present but not identified.
On/Off Target is the percentage of mapped reads that were aligned/not aligned over a target region. This metric is used to ensure the technology amplifies predominantly the targets to which the panel was designed.
Low quality is tracked to ensure the data is worth analyzing. This metric is a general system metric and isn't directly related to this technology.

### Example 4

One benefit of the instant invention is the ability to use Ampliseq.com designer in conjunction with the provided methodology. Adaptors were designed using the composition design approach provided herein, including universal handle-unique tag-gene specific target sequence described in Example 2 above, and targeted to genes using the ONCOMINE™ Focus Research Panel (Thermo Fisher Scientific, Inc.) target sequences and ION AMPLISEQ™ Designer (Thermo Fisher Scientific, Inc). Forward and reverse adaptors described above were utilized comprising

Forward Adaptor:
SEQ ID NO: 1
------A Handle----- ------*UT*------ --Gene Specific--
TCTGTACGGTGACAAGGCG-U-NNNACTNNNTGA-U-GAGGACCGUCGCTUGGT
Rev Adaptor B
SEQ ID NO: 2
CTCTATGGGCAGTCGGTGAT-U-NNNACTNNNTGA-U-CCTTCTGCAUGGTATTCTTTCTCTUCC
-----B Handle------- --------*UT*---- -------Gene Specific-------

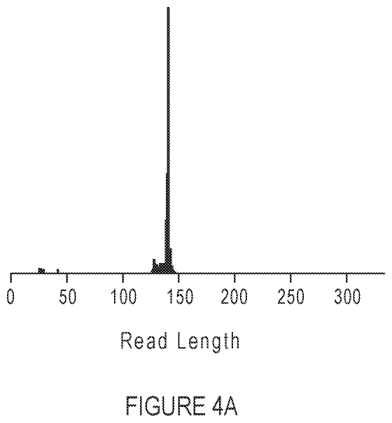

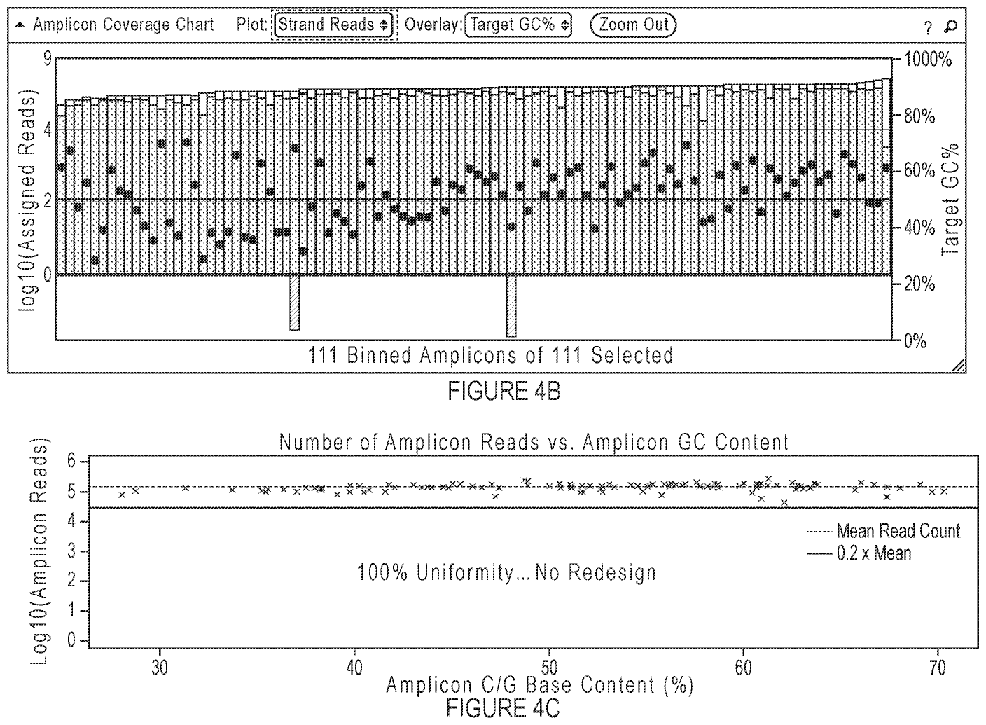

With target sequences specific to targets as in Table A, and adaptors each comprise 4096 unique tag sequences for each gene specific target sequence, resulting in an estimate of 16,777,216 different unique tag combinations for each gene specific target sequence pair. Library was prepared using 20 ng of genomic DNA and ˜1% Acrometrix Oncomine™ Hotspot Control (AOHC) DNA (Thermo Fisher Scientific, Inc.), according to the protocol described above in Example 1C. Prepared library was sequenced using Ion 520/530 Templating/Sequencing kits and instrumentation (Thermo Fisher Scientific, Inc.). Performance with the panel (eg., yield, uniformity) indicates the technology is able to effectively make use of the designer pipeline. See FIG. 4A-4C.

Results using the AOHC DNA (shown in Table 1) demonstrate that, using this protocol, we effectively identify most of the True Positives (71 or 75) present in the AOHC and importantly did not generate any False positives.

TABLE 1

| | | | Oncology | Oncology |
|---|---|---|---|---|
| | Oncology | BRCA | HotSpot | HotSpot |
| | Panel | Panel | Panel | Bidirectional |
| | (Ex 4) | (Ex 5) | (Ex 3) | (ex 6) |
| True Positives | 75 | NA | NA | NA |
| TP in SNP, INDEL | 71; 4 | NA | NA | NA |
| False Negatives | 3 | NA | NA | NA |
| False Positives | 0 | 0 | 0 | 0 |
| Uniformity | 98.60% | 100% | 100% | 100% |
| Low Quality | 15% | 28% | 31% | 26% |
| On Target | 98% | 95% | 96% | 95% |
| # of Families | 4398 | 5208 | 8755 | 6391 |

### Example 5

Adaptors were designed according to the composition design approach provided herein, including universal handle-unique tag-gene specific target sequence described in Example 2 above, and targeted to genes using the BRCA Research Panel (Thermo FisherScientific, Inc.) target sequences and ION AMPLISEQ™ Designer (Thermo Fisher Scientific, Inc). Forward and reverse adaptors described above were utilized comprising

Forward Adaptor:
SEQ ID NO: 1
------A Handle----- ------*UT*------ --Gene Specific--
TCTGTACGGTGACAAGGCG-U-NNNACTNNNTGA-U-GAGGACCGUCGCTUGGT
Rev Adaptor B
SEQ ID NO: 2
CTCTATGGGCAGTCGGTGAT-U-NNNACTNNNTGA-U-CCTTCTGCAUGGTATTCTTTCTCTUCC
-----B Handle------- --------*UT*---- -------Gene Specific-------

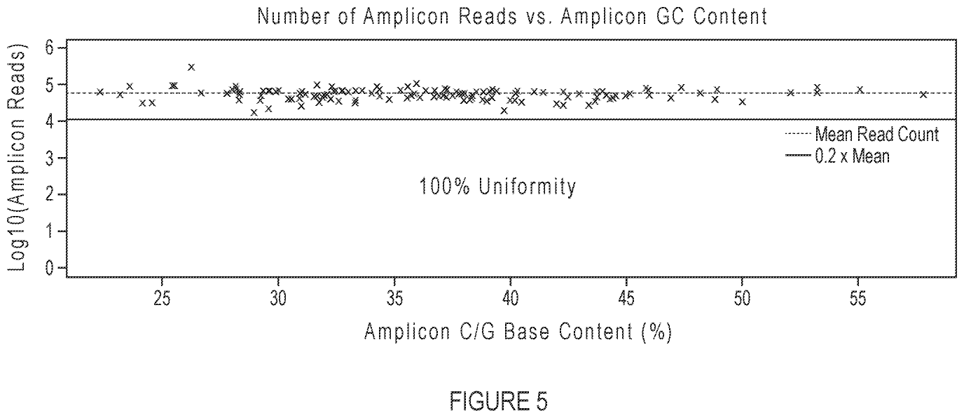

With target sequences specific to targets as in Table C, and adaptors each comprise 4096 unique tag sequences for each gene specific target sequence, resulting in an estimate of 16,777,216 different unique tag combinations for each gene specific target sequence pair. Library was prepared using 20 ng genomic DNA according to the protocol described above in Example 1C Prepared library was sequenced using Ion 520/530 Templating/Sequencing kits and instrumentation (Thermo Fisher Scientific, Inc.). Similar to Example 5, performance (e.g., yield, uniformity) with the panel indicates the technology is able to use the designer pipeline. See FIG. 5 and Table 1.

### Example 6

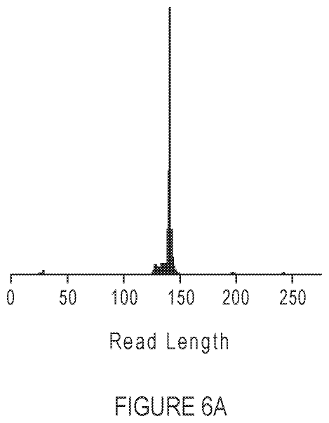

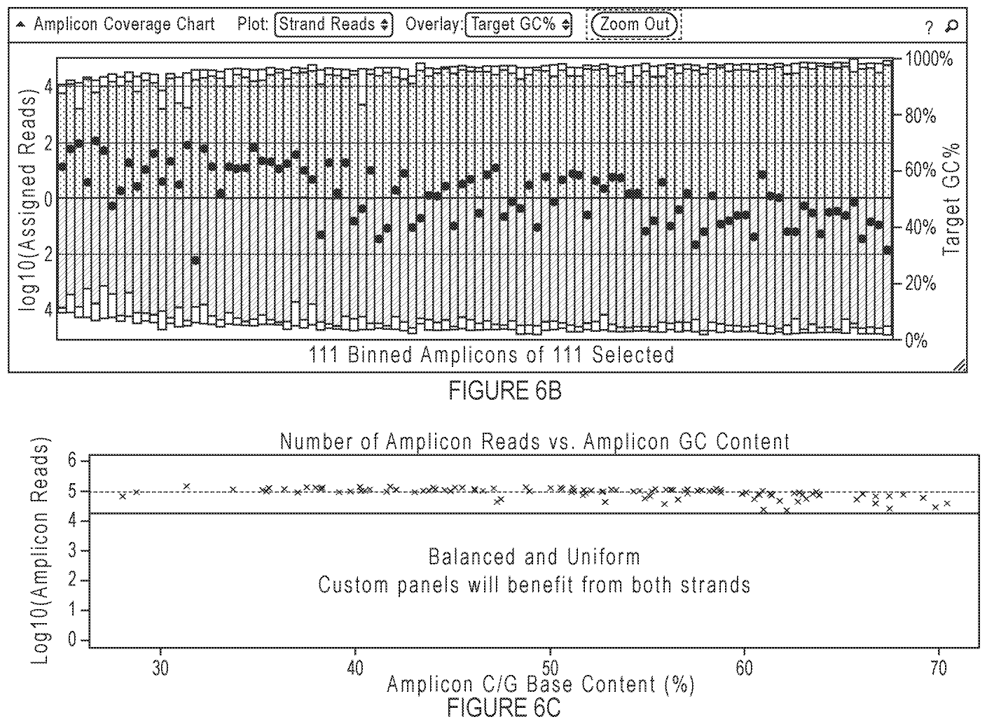

Primers were designed using the composition design approach provided herein and targeted to oncology genes using those of the panel target sequences as described above in Example 4, except that the library amplification step utilized two primer pairs (to put the two universal sequences on each end of amplicons, e.g., an A-universal handle and a P1-universal handle on each end) to enable bi-directional sequencing. Prepared library was sequenced using Ion 520/530 Templating/Sequencing kits and instrumentation (Thermo Fisher Scientific, Inc.). See FIG. 7. Performance (e.g., yield, uniformity) with the instant panel indicates the technology is able to use the designer pipeline and effectively generate sequencing data for both strands of DNA. See FIG. 6A-6C and Table 1.

### Example 7

Primers were designed using the composition design approach provided herein and targeted to a wide variety of oncology target sequences. Forward and reverse adaptors described above were utilized comprising

Forward Adaptor:
SEQ ID NO: 1
------A Handle----- ------*UT*------ --Gene Specific--
TCTGTACGGTGACAAGGCG-U-NNNACTNNNTGA-U-GAGGACCGUCGCTUGGT
Rev Adaptor C
SEQ ID NO: 3
TCTAGTCGGTCAGTCACGG-U-NNNACTNNNTGA-U-CCTTCTGCAUGGTATTCTTTCTCTUCC
-----C Handle------- ------UT------- -------Gene Specific-------

TABLE 2A
Family Generation, Coverage, and Uniformity

| | | | AmpliSeq HD | | | | |
|---|---|---|---|---|---|---|---|
| | | | Median Read | | Median # | | Median # |
| | | | Counts | Uniformity | Families | Molecular | Families |
| Sample | Input | | per Target | (U50) | Size >= 3 | Conversion | Size >= 3 |
| cfDNA 2016B | 20 | ng | 61,939 | 95.9% | 5794 | 48% | 5794 |
| | | | 63,679 | 95.9% | 5879 | 49% | 5879 |
| cfDNA 416G | 20 | ng | 79,004 | 98.6% | 7676 | 64% | 7676 |
| | | | 61,694 | 98.6% | 7322 | 61% | 7322 |
| 0.5% fMM | 6000 | copies | 61,458 | 98.6% | 5466 | 46% | 5466 |
| | | | 62,019 | 98.6% | 5685 | 47% | 5685 |
| 0.1% fMM | 6000 | copies | 70,397 | 98.6% | 6278 | 52% | 6278 |
| | | | 60,879 | 98.6% | 5946 | 50% | 5946 |
| gDNA | 292 | copies | 22,650 | 97.3% | 340 | 57% | 340 |
| | | | 79,746 | 98.6% | 354 | 59% | 354 |

TABLE 2B
Sensitivity, Specificity, and FPs/lib, Hot Spots Only

| | | | AmpliSeq HD | | |
|---|---|---|---|---|---|
| Sample | Input | | Sensitivity (%) | Specificity (%) | FP |
| cfDNA 2016B | 20 | ng | | 100.00 | 0 |
| | | | | 99.70 | 1 |
| cfDNA 416G | 20 | ng | | 100.00 | 0 |
| | | | | 100.00 | 0 |
| 0.5% allelic | 6000 | copies | 100.0 | 100.00 | 0 |
| Frequency | | | 100.00 | | 0 |
| 0.1% allelic | 6000 | copies | 85.14 | 100.00 | 0 |
| Frequency | | | 94.60 | 100.00 | 0 |
| gDNA | 292 | copies | | 100.00 | 0 |
| | | | | 100.00 | 0 |

With target sequences specific to targets as in Table D and adaptors each comprise 4096 unique tag sequences for each gene specific target sequence, resulting in an estimate of 16,777,216 different unique tag combinations for each gene specific target sequence pair. Samples containing 19.8 ng of cell free DNA and 0.2 ng of total RNA were processed as described in example 1D, starting with the optional reverse transcriptase step. Total RNA for some samples listed contained 5 spiked in fusion constructs. See Table D. Prepared library was sequenced using Ion 520/530 Templating/Sequencing kits and instrumentation (Thermo Fisher Scientific, Inc.). Performance (e.g., yield, uniformity, molecular conversion, sensitivity) with the instant panel indicates the technology can efficiently convert input DNA into library and detect mutations present at frequencies as low as 0.1% to 0.5%. See Table 2A-2B. Additionally, results confirm the technology can efficiently convert input DNA and cDNA into library and detect fusions present at frequencies of ˜1%. See Table 3A-3B.

TABLE 3A
Fusions

| | | |
|---|---|---|
| LRIG3-ROS1 | EZR-ROS1 | KLC1-ALK |
| CCDC6-RET | GOPC-ROS1 | SDC4-ROS1 |
| CD74-ROS1 | HIP1-ALK | SLC34A2-ROS1 |
| CUX1-RET | KIF5B-ALK | TPM3-ROS1 |
| EML4-ALK | KIF5B-RET | TPR-ALK |

TABLE 3B
Family Generation, Coverage, and Uniformity (No Activation)

| Sample | Input | FP | U50 | Conversion |
|---|---|---|---|---|
| cfDNA 5022 | 10 ng | 0 (343) | 98.5 | 44% |
| cfDNA 5022 + | 10 ng | 0 (343) | 99.25 | 51% |
| total RNA | | 2 (323) | | |
| cfDNA 5022 + | 10 ng | 0 (343) | 98.5 | 50% |
| Trifusion | | 1 (323) | | |
| gDNA | 10 ng | 0 (343) | 93.98 | 45% |
| | | 2 (323) | | |
| gDNA + | 10 ng | 0 (343) | 93.98 | 54% |
| total RNA | | 0 (323) | | |
| gDNA + | 10 ng | 1 (343) | 95.49 | 53% |
| Trifusion | | 1 (323) | | |

### Example 8

Primers were designed using the composition design approach provided herein and targeted to genes using those of short tandem repeats (STRs), which are useful for high resolution genotyping and analysis of complex mixtures. Forward and reverse adaptors described above were utilized comprising

Forward Adaptor:
SEQ ID NO: 1
------A Handle----- ------*UT*------ --Gene Specific--
TCTGTACGGTGACAAGGCG-U-NNNACTNNNTGA-U-GAGGACCGUCGCTUGGT
Rev Adaptor E
SEQ ID NO: 5
TGACAAGGCGTAGTCACGG-U-NNNACTNNNTGA-U-CCTTCTGCAUGGTATTCTTTCTCTUCC
-----E Handle------- ------UT------- -------Gene Specific-------

With target sequences specific to targets as in Table E and adaptors each comprise 4096 unique tag sequences for each gene specific target sequence, resulting in an estimate of 16,777,216 different unique tag combinations for each gene specific target sequence pair. Samples containing 1 to 10 ng of genomic DNA were processed as described in example 1D without the optional reverse transcriptase step. Prepared library was sequenced using Ion 520/530 Templating/Sequencing kits and instrumentation (Thermo Fisher Scientific, Inc.). Performance (e.g., yield, uniformity) with the instant panel indicates that even challenging STR targets (which are often shortened by 1 or more repeats during amplification) can be efficiently converted into a library. Results were consistent across titration levels of input DNA. See Table 4. When results were compared to standard operating procedure according to manufacturer instructions using Torrent Suite Molecular Diagnostics plugin to evaluate the same targets, results generated using compositions and methods provided herein yielded more consistent signal over each of the repeat regions, with less stutter (data not shown).

TABLE 4

| | | Median | | Half-Double | 80% |
|---|---|---|---|---|---|
| | | Read | Median # | Uniformity | Uniformity |
| Barcode | Input | Counts per | Families | (Families | (Families |
| Name | DNA | Target | Size >= 3 | Size >= 3) | Size >= 3) |
| BC_0102 | 1 ng | 37,727 | 257 | 77.78% | 63.89% |
| BC_0105 | 2 ng | 35,056 | 412 | 83.33% | 63.89% |
| BC_0108 | 5 ng | 32,478 | 1021 | 80.56% | 69.44% |
| BC_0120 | 10 ng | 30,915 | 1646 | 86.11% | 63.89% |

### Example 9

Primers were designed using the composition design approach provided herein and targeted to oncology genes target sequences as described above in Example 6, where two primer pairs were utilized in library amplification (to put the two universal sequences on each end of amplicons, e.g., an A-universal handle and a P1-universal handle on each end) to enable bi-directional sequencing. Library preparation was carried out on samples containing spiked in AOHC control as described according to methods of Example 1E above without optional RT step. See FIG. 7. Prepared library was sequenced using Ion 520/530 Templating/Sequencing kits and instrumentation (Thermo Fisher Scientific, Inc.), then analyzed separately for unidirectional sequence results as well as results analyzed from bidirectional sequencing. Performance (e.g., yield, uniformity, sensitivity) with the instant panel indicates the technology is able to use the designer pipeline and effectively generate sequencing data for both strands of DNA, and bidirectional sequence analysis results in reduction of indel False Positives measured. See Table 5.

TABLE 5

| | Bidirectional, | Bidirectional, |
|---|---|---|
| | Analyzed | Analyzed |
| | Unidirectional | Bidirectional |
| True Positives | 67 | 67 |
| Sensitivity | 91.8 | 91.8 |
| TP in SNP, INDEL | 65; 2 | 65; 2 |
| False Negatives | 6 | 6 |
| False Positives in SNP, INDEL | 1:2 | 1:0 |

### Example 10

For each of the Ion barcode adaptors, a single barcode is included in an A adapter. Addition of a second set of barcodes on the P1 adapter can effectively reduce the level of contamination artifacts in results by filtering out identified contamination reads. Primers were designed using the composition design approach provided herein and targeted to a wide variety of oncology target sequences. Samples containing 20 ng of genomic DNA were processed similarly to those described in Example 7 above and using the method of example 1D, however, additionally barcoded P1 adapters were also utilized, wherein a barcode 12mer sequence was inserted into the P1 adapter sequence of the reverse adaptor. Sample containing genomic DNA for library preparation was processed with barcode 8 in both A and P1 adapters. Additional samples were also processed with barcodes 1, 2, 3, 4, 5, 6, 7 and 9 (each in both P1 and A barcoded adapters), but without genomic DNA. Performance (e.g., yield, uniformity, Conversion) with the instant panel indicates that additional barcodes can effectively identify contamination. See Table 6.

TABLE 6

| Reverse Barcode | Reads Detected | % Total |
|---|---|---|
| bc1 | 332 | 0.001% |
| bc2 | 54 | 0.000% |
| bc3 | 261 | 0.001% |
| bc4 | 481 | 0.001% |
| bc5 | 9,908 | 0.019% |
| bc6 | 8,532 | 0.016% |
| bc7 | 2,656 | 0.005% |
| bc8 | 52,089,480 | 99.941% |
| bc9 | 1,403 | 0.003% |
| bc10 | 7,131 | 0.014% |

### Example 11

In another specific implementation, adaptors were prepared as described in example 2A for targets of the ONCOMINE™ Focus Assay, as in Table B, as well as described in example 6 with target sequences specific to targets as in Table D and adaptors each comprise 4096 unique tag sequences for each gene specific target sequence, resulting in an estimate of 16,777,216 different unique tag combinations for each gene specific target sequence pair. Forward and reverse adaptors utilized comprising

Forward Adaptor:
SEQ ID NO: 1
------A Handle------ ------*UT*---- ---Gene Specific--
TCTGTACGGTGACAAGGCG-U-NNNACTNNNTGA-U-GAGGACCGUCGCTUGGT
Rev Adaptor I:
SEQ ID NO: 1705
TGACAAGGCGTAGTCACGG-U-NNNACTNNNTGA-U-CCTTCTGCAUGGTATTCTTTCTCTUCC
-----T Handle---------------*UT*---- -------Gene Specific-------

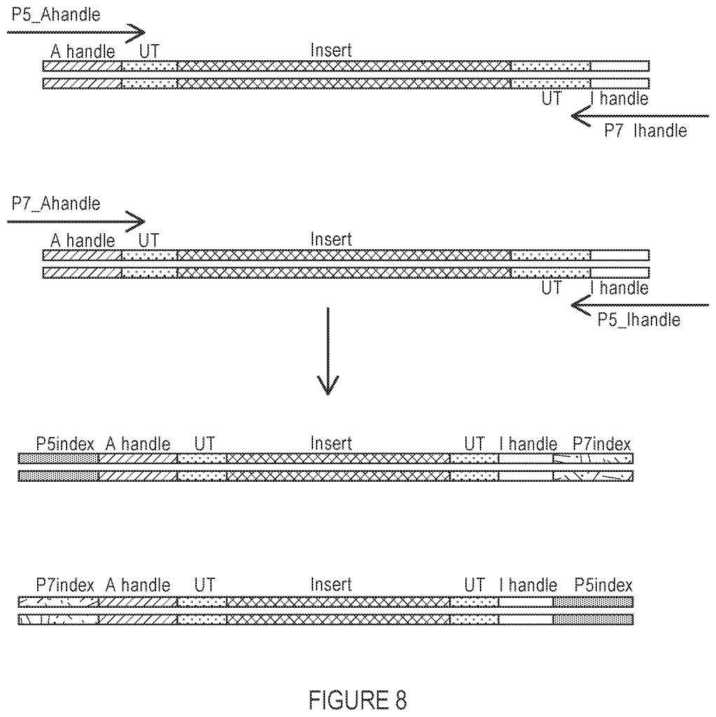

Preparation of library was carried out according to the method described above for 1F. See also FIG. 8. The workflow has been adapted to use amplification primers to enable libraries to carry out sequencing runs on the Illumina platform. The design (shown schematically in FIG. 8) contains: (1) P5 grafting primer region; (2) P5 index (A-H) region; (3) P5 sequencing/index read primer region; (4) A-handle region; (5) UT region; (6) gene specific insert; (7) UT region; (8) I-handle region; (9) P7 sequencing/index read primer region; (10) P7 index (1-12) region; and (11) P7 grafting primer region. 3 libraries were made with an oncology panel comprising targets of Table D having idex5-01-idex7-5, idex5-02-index7-6 and idex5-7-idex7-7 respectively. 2 libraries were made with Focus panel comprising targets of Table B having idex5-01-idex7-5, and idex5-7-idex7-7 respectively. See Table F. All libraries are made with 19.6 ng of g24385 with 0.4 ng spike-in AOHC so we could detect 0.1% allele frequency.

TABLE F

| Name | Sequence (5′ to 3′) | SEQ ID |
|---|---|---|
| 5-01-Ah | AATGATACGGCGACCACCGAGATCTACAC AGCGCTAG TCGTCGGCAGCGTC | 1706 |
| | AGATGTGTATAAGAGACAG TCTGTACGGTGACAAGGCGT | |
| 5-02-Ah | AATGATACGGCGACCACCGAGATCTACAC GATATCGA TCGTCGGCAGCGTC | 1707 |
| | AGATGTGTATAAGAGACAG TCTGTACGGTGACAAGGCGT | |
| 5-07-Ah | AATGATACGGCGACCACCGAGATCTACAC ACATAGCG TCGTCGGCAGCGTC | 1708 |
| | AGATGTGTATAAGAGACAG TCTGTACGGTGACAAGGCGT | |
| 5-08-Ah | AATGATACGGCGACCACCGAGATCTACAC GTGCGATA TCGTCGGCAGCGTC | 1709 |
| | AGATGTGTATAAGAGACAG TCTGTACGGTGACAAGGCGT | |
| 5-09-Ah | AATGATACGGCGACCACCGAGATCTACAC CCAACAGA TCGTCGGCAGCGTC | 1710 |
| | AGATGTGTATAAGAGACAG TCTGTACGGTGACAAGGCGT | |
| 5-010-Ah | AATGATACGGCGACCACCGAGATCTACAC TTGGTGAG TCGTCGGCAGCGTC | 1711 |
| | AGATGTGTATAAGAGACAG TCTGTACGGTGACAAGGCGT | |
| 5-013-Ah | AATGATACGGCGACCACCGAGATCTACAC AACCGCGG TCGTCGGCAGCGTC | 1712 |
| | AGATGTGTATAAGAGACAG TCTGTACGGTGACAAGGCGT | |
| 5-014-Ah | AATGATACGGCGACCACCGAGATCTACAC GGTTATAA TCGTCGGCAGCGTC | 1713 |
| | AGATGTGTATAAGAGACAG TCTGTACGGTGACAAGGCGT | |
| 01-Ih | AATGATACGGCGACCACCGAGATCTACAC AGCGCTAG TCGTCGGCAGCGTC | 1714 |
| | AGATGTGTATAAGAGACAG TGACAAGGCGTAGTCACGGT | |
| 02-Ih | AATGATACGGCGACCACCGAGATCTACAC GATATCGA TCGTCGGCAGCGTC | 1715 |
| | AGATGTGTATAAGAGACAG TGACAAGGCGTAGTCACGGT | |
| 5-07-Ih | AATGATACGGCGACCACCGAGATCTACAC ACATAGCG TCGTCGGCAGCGTC | 1716 |
| | AGATGTGTATAAGAGACAG TGACAAGGCGTAGTCACGGT | |
| 5-08-Ih | AATGATACGGCGACCACCGAGATCTACAC GTGCGATA TCGTCGGCAGCGTC | 1717 |
| | AGATGTGTATAAGAGACAG TGACAAGGCGTAGTCACGGT | |
| 5-09-Ih | AATGATACGGCGACCACCGAGATCTACAC CCAACAGA TCGTCGGCAGCGTC | 1718 |
| | AGATGTGTATAAGAGACAG TGACAAGGCGTAGTCACGGT | |
| 5-010-Ih | AATGATACGGCGACCACCGAGATCTACAC TTGGTGAG TCGTCGGCAGCGTC | 1719 |
| | AGATGTGTATAAGAGACAG TGACAAGGCGTAGTCACGGT | |
| 5-013-Ih | AATGATACGGCGACCACCGAGATCTACAC AACCGCGG TCGTCGGCAGCGTC | 1720 |
| | AGATGTGTATAAGAGACAG TGACAAGGCGTAGTCACGGT | |
| 5-014-Ih | AATGATACGGCGACCACCGAGATCTACAC GGTTATAA TCGTCGGCAGCGTC | 1721 |
| | AGATGTGTATAAGAGACAG TGACAAGGCGTAGTCACGGT | |
| 7-1-Ah | CAAGCAGAAGACGGCATACGAGAT ATATTCAC GTCTCGTGGGCTCGG | 1722 |
| | AGATGTGTATAAGAGACAG TCTGTACGGTGACAAGGCGT | |
| 7-2-Ah | CAAGCAGAAGACGGCATACGAGAT GCGCCTGT GTCTCGTGGGCTCGG | 1723 |
| | AGATGTGTATAAGAGACAG TCTGTACGGTGACAAGGCGT | |
| 7-3-Ah | CAAGCAGAAGACGGCATACGAGAT ACTCTATG GTCTCGTGGGCTCGG | 1724 |
| | AGATGTGTATAAGAGACAG TCTGTACGGTGACAAGGCGT | |
| 7-4-Ah | CAAGCAGAAGACGGCATACGAGAT GTCTCGCA GTCTCGTGGGCTCGG | 1725 |
| | AGATGTGTATAAGAGACAG TCTGTACGGTGACAAGGCGT | |
| 7-5-Ah | CAAGCAGAAGACGGCATACGAGAT AGTAGAGA GTCTCGTGGGCTCGG | 1726 |
| | AGATGTGTATAAGAGACAG TCTGTACGGTGACAAGGCGT | |
| 7-6-Ah | CAAGCAGAAGACGGCATACGAGAT GACGAGAG GTCTCGTGGGCTCGG | 1727 |
| | AGATGTGTATAAGAGACAG TCTGTACGGTGACAAGGCGT | |
| 7-7-Ah | CAAGCAGAAGACGGCATACGAGAT AGACTTGG GTCTCGTGGGCTCGG | 1728 |
| | AGATGTGTATAAGAGACAG TCTGTACGGTGACAAGGCGT | |
| 7-8-Ah | CAAGCAGAAGACGGCATACGAGAT GAGTCCAA GTCTCGTGGGCTCGG | 1729 |
| | AGATGTGTATAAGAGACAG TCTGTACGGTGACAAGGCGT | |
| 7-9-Ah | CAAGCAGAAGACGGCATACGAGAT AATTCTGC GTCTCGTGGGCTCGG | 1730 |
| | AGATGTGTATAAGAGACAG TCTGTACGGTGACAAGGCGT | |
| 7-10-Ah | CAAGCAGAAGACGGCATACGAGAT GGCCTCAT GTCTCGTGGGCTCGG | 1731 |
| | AGATGTGTATAAGAGACAG TCTGTACGGTGACAAGGCGT | |
| 7-1-Ah | CAAGCAGAAGACGGCATACGAGAT ATCTTAGT GTCTCGTGGGCTCGG | 1732 |
| | AGATGTGTATAAGAGACAG TCTGTACGGTGACAAGGCGT | |
| 7-12-Ah | CAAGCAGAAGACGGCATACGAGAT GCTCCGAC GTCTCGTGGGCTCGG | 1733 |
| | AGATGTGTATAAGAGACAG TCTGTACGGTGACAAGGCGT | |
| 7-1-Ih | CAAGCAGAAGACGGCATACGAGAT ATATTCAC GTCTCGTGGGCTCGG | 1734 |
| | AGATGTGTATAAGAGACAG TGACAAGGCGTAGTCACGGT | |
| 7-2-Ih | CAAGCAGAAGACGGCATACGAGAT GCGCCTGT GTCTCGTGGGCTCGG | 1735 |
| | AGATGTGTATAAGAGACAG TGACAAGGCGTAGTCACGGT | |
| 7-3-Ih | CAAGCAGAAGACGGCATACGAGAT ACTCTATG GTCTCGTGGGCTCGG | 1736 |
| | AGATGTGTATAAGAGACAG TGACAAGGCGTAGTCACGGT | |
| 7-4-Ih | CAAGCAGAAGACGGCATACGAGAT GTCTCGCA GTCTCGTGGGCTCGG | 1737 |
| | AGATGTGTATAAGAGACAG TGACAAGGCGTAGTCACGGT | |
| 7-5-Ih | CAAGCAGAAGACGGCATACGAGAT AGTAGAGA GTCTCGTGGGCTCGG | 1738 |
| | AGATGTGTATAAGAGACAG TGACAAGGCGTAGTCACGGT | |
| 7-6-Ih | CAAGCAGAAGACGGCATACGAGAT GACGAGAG GTCTCGTGGGCTCGG | 1739 |
| | AGATGTGTATAAGAGACAG TGACAAGGCGTAGTCACGGT | |
| 7-7-Ih | CAAGCAGAAGACGGCATACGAGAT AGACTTGG GTCTCGTGGGCTCGG | 1740 |
| | AGATGTGTATAAGAGACAG TGACAAGGCGTAGTCACGGT | |
| 7-8-Ih | CAAGCAGAAGACGGCATACGAGAT GAGTCCAA GTCTCGTGGGCTCGG | 1741 |
| | AGATGTGTATAAGAGACAG TGACAAGGCGTAGTCACGGT | |
| 7-9-Ih | CAAGCAGAAGACGGCATACGAGAT AATTCTGC GTCTCGTGGGCTCGG | 1742 |
| | AGATGTGTATAAGAGACAG TGACAAGGCGTAGTCACGGT | |
| 7-10-Ih | CAAGCAGAAGACGGCATACGAGAT GGCCTCAT GTCTCGTGGGCTCGG | 1743 |
| | AGATGTGTATAAGAGACAG TGACAAGGCGTAGTCACGGT | |
| 7-11-Ih | CAAGCAGAAGACGGCATACGAGAT ATCTTAGT GTCTCGTGGGCTCGG | 1744 |
| | AGATGTGTATAAGAGACAG TGACAAGGCGTAGTCACGGT | |
| 7-12-Ih | CAAGCAGAAGACGGCATACGAGAT GCTCCGAC GTCTCGTGGGCTCGG | 1745 |
| | AGATGTGTATAAGAGACAG TGACAAGGCGTAGTCACGGT | |

To mimic low level of mutant variants (0.10%) presence in DNA samples, we used purified genomic DNA and spiked in small quantity of AcroMetrix Oncology Hotpot Control plasmid. These samples are used as our control samples for the purpose of demonstrating the library preparation method and assessing the sensitivity and specificity for low levels mutant variants detection by this assay method. Bioanalyzer results matched library structure designs, and yield and purity of libraries were on par with those prepared on other methods described above. Similar successful sequencing results were generated with each of the adaptor pairings.

A MiSeq sequencing run successfully generated clusters, and produced sequencing and indexing reads. Sequencing results of the panel run on the Illumina MiSeq indicate similar performance as compared to the standard AmpliSeq HD version run on the Ion S5 using a 540 chip. See Table 7.

TABLE 7

| | MiSeq | S5 540 |
|---|---|---|
| Raw Read Accuracy (%) | 99.31 | 99.27 |
| Mapped Reads | 12,994,280 | 17,855,575 |
| Mean Depth | 46,674 | 62,429 |
| On-Target (%) | 98.91 | 98.64 |
| coverageAnalysis Uniformity (%) | 97.86 | 97.98 |
| Half-Double Uniformity (%) | 86.62 | 83.64 |
| 0.1% MegaMix TP | 140 | 138 |
| 0.1% MegaMix FN | 11 | 13 |
| 0.1% MegaMix FP | 58 | 38 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

TABLE A

| Primer | | Primer Sequence | Primer | | Primer Sequence |
|---|---|---|---|---|---|
| Name | SEQ ID | (target of primer A) | Name | SEQ ID | (target seq of primer B) |
| F1 | 8 | GCTCCCAGGCACUTGATGAUAC | R1 | 104 | ACAGAAUCACAUGCCACACAGT |
| F2 | 9 | TGTTGCCATTUCAGGGTTTCUGA | R2 | 105 | TTCCTTCUAAAAGGCCATGAAGATCUG |
| F3 | 10 | ACCAAUGCGAGGAAGAAAAACAAUC | R3 | 106 | TCTGAAGAACAUGTGUGAGCACA |
| F4 | 11 | TCTTCAGAAUCTTGTTGGCUGCAT | R4 | 107 | CCCAACCCACAUTTCCTTTATAGATGTUT |
| F5 | 12 | AGAGTTGCAUCCTTCCCTTCUCT | R5 | 108 | TGTTCCAACAGGAUCTGUCCAAAA |
| F6 | 13 | CTGGCAUGACGCAGTTTCTUC | R6 | 109 | CCAGTGTCTGUCCTTGCCTTUC |
| F7 | 14 | GATCAAAGAGACGAAGUCTCTUGCA | R7 | 110 | CUATGACAAGAAAAUGGACACCAACAAG |
| F8 | 15 | CTTGCCUAGACAGCACCGUAAT | R8 | 111 | AGGAGGAUAAAGACCTGGUCCAT |
| F9 | 16 | TGGTTTCTGGUGGGACCATTAUG | R9 | 112 | GTCCTCUGGATCTCTTCAUGCA |
| F10 | 17 | TTGAAAGAGAACACACUTACTCUCCAC | R10 | 113 | CTGAGACATTCCUATGTCCTGCUC |
| F11 | 18 | CTCTACCAGAGUTAATCAACTGAUGCA | R11 | 114 | TUGAUACAAAACAAGCCCACGAACT |
| F12 | 19 | TATCAACTGTCCUTGTTGGCAAAUCA | R12 | 115 | CCAGCCTAATCTUGTTTTTCTTATGTTCUG |
| F13 | 20 | GCAAATGACTUGCTATTATTGAUGGCA | R13 | 116 | GTTATAGATGGUGAAACCTGTTTGTUGG |
| F14 | 21 | GTGGGATCATAUTCATCTACAAAGUGGT | R14 | 117 | GATTACTGGTTUCCAACAGGTTCTUG |
| F15 | 22 | GGAGGTCAUGGCATCGAGUT | R15 | 118 | TATGGTCTUGGACATCCAGGAUCT |
| F16 | 23 | GTGAGGCAGUCTTTACUCACCT | R16 | 119 | TAGGAAATGCAUTTCCTTTCTUCCCA |
| F17 | 24 | GGGAAATGUGAGCCCTUGAGAT | R17 | 120 | CCTGTGGCUGTCAGTATTUGGA |
| F18 | 25 | ACTCTTGCUCCTTCCATCCTUG | R18 | 121 | GTTCATCCUGCTGGAGCUCAT |
| F19 | 26 | CCCAAUGCAGCGAACAATGTUC | R19 | 122 | GTAGCTGCTGAAAAUGTAACTTTGTAUCC |
| F20 | 27 | GATCAGGGCUTCCAUGAGGAAA | R20 | 123 | ACTCTGTAGGCUGCAGTTCUCA |
| F21 | 28 | CTCAAGAGUGAGCCACTTCTUACC | R21 | 124 | CTCCTCTTGUCTTCTCCTTUGCA |
| F22 | 29 | AGAATAAAACACAUACAAGTTGGAAATTTCUGG | R22 | 125 | CTTGTGAGTGGAUGGGTAAAACCUAT |
| F23 | 30 | ACCUGAGCCAAGGACTTTUACC | R23 | 126 | CGGACTGAAAGUATAACCTTCTTCTTUCC |
| F24 | 31 | TGTCAATTAGCUGGAACATCUGAAACT | R24 | 127 | GCATGTGAACAUTCTGCTTTTCAUGG |
| F25 | 32 | GTGCCCTATUACCTCAATCAUCCT | R25 | 128 | ACGCCTTCACCUTTAACACCUC |
| F26 | 33 | CCAGACAGAAAAGCGGCUGTUA | R26 | 129 | ACTTGGGAGGUATCCACAUCCT |
| F27 | 34 | CCTAGTAGAATGTTUACTACCAAATGGAAUGA | R27 | 130 | AGATTCATCTUGAAGAAGTTGAUGGAGG |
| F28 | 35 | TTTTTGAUGAAACAAGACGACTTTGUG | R28 | 131 | GAATAGGATATTGUATCATACCAATTTCUCGAT |
| F29 | 36 | CACAGCUACACCATATATGAAUGGAGA | R29 | 132 | CAGCATTTGACTTUACCTTATCAATGTCUC |
| F30 | 37 | GATCTATGTUCGAACAGGTATCUACCATG | R30 | 133 | ACTGCTAAACACTAAUATAACCTTTGGAAAUAT |
| F31 | 38 | GGGAAGAAAAGUGTTTTGAAATGTGTUT | R31 | 134 | CATTTTTCCAGATACUAGAGTGTCTGTGUA |
| F32 | 39 | TTTGAATCTTUGGCCAGTACCUCA | R32 | 135 | CATAAGAGAGAAGGUTTGACTGCCAUAAA |
| F33 | 40 | GACTAGCUAGAGACAATGAATUAAGGGAAAA | R33 | 136 | GAATCTCCATTTUAGCACTTACCTGUGA |
| F34 | 41 | CTGAGATGCACAAUAAAACAGTUAGCC | R34 | 137 | AGAATGTCAGTUAAGTTAATGAGCTTTUCCAT |
| F35 | 42 | GACAUTCUCAAACAGGAGAAGAAGGA | R35 | 138 | GCTTGATUCCAAGGACCATGATCUG |
| F36 | 43 | TCTTTTCTCAAGUTGGCCTGAAUCA | R36 | 139 | CAATTCCCAAAAUGAAGGTAGCUACAC |
| F37 | 44 | CAATCTTTTGAUGACATTGCATACATUCGA | R37 | 140 | GGAAGATCCAAUCCATTTTTGTTGUCC |
| F38 | 45 | GTATGCAGGCAUCCTCAGCUA | R38 | 141 | CGGGAAGCGGGAGAUCTUG |
| F39 | 46 | CUGGUGACCGAGGACAACGT | R39 | 142 | GGCGTCCUACTGGCAUGA |
| F40 | 47 | GTCCUGGGAGTCUCAGGACA | R40 | 143 | CCTUCAGCAGCTUGAAGAGCT |
| F41 | 48 | CCAGTTACCUGTCCTGGTCAUT | R41 | 144 | GGAAACTCCCAUCTTGAGTCAUAAGG |
| F42 | 49 | AGTGAAAAACAAGCUCTCATGTCUGA | R42 | 145 | CATGTGTCCAGUGAAAATCCUCACT |
| F43 | 50 | GGAAAAATTGUGAAGATCTGTGACTTUGG | R43 | 146 | CTGACTUTAGAGATTAAAGUGAAGGAGGAT |
| F44 | 51 | AGCACTCTGACAUATGGCCATUT | R44 | 147 | CCTGGACAAAAAUACCAATCTATTGUGG |
| F45 | 52 | GGCACGGTUGAATGTAAGGCTUA | R45 | 148 | ACTGATATGGUAGACAGAGCCUAAACAT |
| F46 | 53 | CCCACAGAAACCCAUGTAUGAAGT | R46 | 149 | ACTGACCAAAACUCAGCCTGUT |
| F47 | 54 | TUGACAGAACGGGAAGCCCUCAT | R47 | 150 | CCTGACAGACAAUAAAAGGCAGCUT |
| F48 | 55 | CCTTACTCAUGGTCGGAUCACAA | R48 | 151 | GTTGAAACUAAAAATCCTTUGCAGGACT |
| F49 | 56 | CCAATATTATGGAUCCCAACTGCCUA | R49 | 152 | ACATTCTGAAGCAGCUTGGAGTUT |
| F50 | 57 | GCTACTTTGAUTTCTCCACTUCCAAC | R50 | 153 | GAGGAGATTGAAAAUCTTCCTGCCUT |
| F51 | 58 | AAAGGCAUGGAGCATCTGUACA | R51 | 154 | TTGGTCCGUCTCCUCCACGG |
| F52 | 59 | GTTATGTCCUCATTGCCCUCAACA | R52 | 155 | CTTCAGTCCGGUTTTATTTGCATCAUAG |
| F53 | 60 | CGAGGGCAAAUACAGCTTUGGT | R53 | 156 | GACTCTCCAAGAUGGGATACUCCA |
| F54 | 61 | TGATGGAGAUGTGATAATTUCAGGAAACA | R54 | 157 | CGGTGACTUACTGCAGCTGTUT |
| F55 | 62 | GAGACATGCAUGAACATTTTTCUCCA | R55 | 158 | TCCAGACCAGGGUGTTGTTTUC |
| F56 | 63 | GCCTCTUACACCCAGUGGAGAA | R56 | 159 | TGTGCCAGGGACCUTACCTTAUA |
| F57 | 64 | CCTTCTCUCTCTGTCAUAGGGACT | R57 | 160 | CACAGCAAAGCAGAAACUCACAUC |
| F58 | 65 | CTCCAGGAAGCCUACGTGAUG | R58 | 161 | TGTGTUCCCGGACATAGUCCA |
| F59 | 66 | CAGGAACGUACTGGUGAAAACAC | R59 | 162 | GAAAATGCUGGCTGACCUAAAGC |
| F60 | 67 | GCTTGTAAGUGCCCGAAGTGUA | R60 | 163 | CACAACCCACUGAGGTATATGTATAGGUAT |
| F61 | 68 | CGCAGTGCUAACCAAGTTCTTUC | R61 | 164 | CCATGGTTAAATAAAAUGCCACTTACTGUT |
| F62 | 69 | CAGTCAAGGUTGCTGATTTUGGT | R62 | 165 | CTTGGTGGUAAACTTTTGAGTTUGCA |
| F63 | 70 | CGAATCGCUACCCTGCTGUT | R63 | 166 | CCAAGCCUCATGGUGCCAT |
| F64 | 71 | TACCGAUACCGUGCGGG | R64 | 167 | TACUGGCAGCAAGUGCCCAG |
| F65 | 72 | CTGTCCUCCACAGGCATTTTUG | R65 | 168 | CCCTCACUCACAGCACATAGUC |
| F66 | 73 | CCATCCCUGACTGTGAGAUCAA | R66 | 169 | CCAGGUACGCCTCCAGAUGA |
| F67 | 74 | TCAGTGGAAAAAUAGCCTCAATTCTUACC | R67 | 170 | CTTCATGAAGACCUCACAGTAAAAAUAGGT |
| F68 | 75 | TATTATGACTUGTCACAATGUCACCACAT | R68 | 171 | GACTCGAGTGAUGATTGGGAGATUC |
| F69 | 76 | TTCCUTAGTCTTTCTTUGAAGCAGCA | R69 | 172 | AGATGCTCUGAGAAAGGCATUAGAAAG |
| F70 | 77 | TCTGACUCCACGAGAACTTGAUCATA | R70 | 173 | TATTGTTAACCUTGCAGAATGGUCGA |
| F71 | 78 | CAAGGCAUAAAAGCTGGGAAAUAGG | R71 | 174 | CUACCTGCCUACGCAACAAGAT |
| F72 | 79 | GGCTAUGGCACCUGCAACT | R72 | 175 | GGGACCUCAGATGTGCTGTUG |
| F73 | 80 | CCACAGAUCCACTGUGCGAC | R73 | 176 | GTGGCTTGUGGGCAAACTUG |
| F74 | 81 | CCATCCTGACCUGGTATGGUCA | R74 | 177 | CCTGCTUCAGGACGTTGAACUC |
| F75 | 82 | CAGCTCGTUCATCGGGACUT | R75 | 178 | ACCTGGCUCCTCTTCACGUA |
| F76 | 83 | CCTCCTTCCUAGAGAGTTAGAGUAACT | R76 | 179 | CACCCACACUTACACATCACTTUG |
| F77 | 84 | CACTGTGTTACUGCCATCGACTUA | R77 | 180 | TCGAGATTUAGCAGCCAGAAATGTUT |
| F78 | 85 | GATTCAATCAAACUGCAGAGTATTUGGG | R78 | 181 | TGATCTGGUGTCAGAGAUGGAGAT |
| F79 | 86 | GGCTTCTTGGUCGTGTTCTUCA | R79 | 182 | CUAGCGCCUGGAAGAGAAAAGGAGAT |
| F80 | 87 | CCCAGCGUCCTCAAAAGTUACA | R80 | 183 | CCCTCCACAAUCATTCCTGUGT |
| F81 | 88 | CTCTACGUCTCCUCCGACCA | R81 | 184 | CTTATTTATTGGTCUCTCATTCTCCCAUCC |
| F82 | 89 | CCTGTACTGGUGGATGTCCUCA | R82 | 185 | GCCTGTUGGACATCCTGGAUAC |
| F83 | 90 | CGCCAGGCUCACCTCTAUAG | R83 | 186 | AGGAGCGAUGACGGAATAUAAGC |
| F84 | 91 | TGTCTTTGCUGATGTTTCAAUAAAAGGAA | R84 | 187 | AGTTAAGGACTCUGAAGATGTACCTAUGG |
| F85 | 92 | CATGTACTGGUCCCTCATUGCA | R85 | 188 | GTAATAATCCAGACUGTGTTTCTCCCUT |
| F86 | 93 | TACCTCTATTGTUGGATCATATTCGUCCA | R86 | 189 | TATTATAAGGCCUGCTGAAAATGACUGAAT |
| F87 | 94 | CAGACACTGUACAAGCTCUACGA | R87 | 190 | GAAUAAAGAGGAGCAGGTUGAGGAA |
| F88 | 95 | CTCTGUCACAGTGGATUCGAGA | R88 | 191 | CAACATGACGAAGAUGGCAAACTUC |
| F89 | 96 | TCCUTCCATAGUGACCAAGACCA | R89 | 192 | GGGTACAUACAAAGCAGTCTGUGT |
| F90 | 97 | GGTTCCATUGGTAGCTGGUGAT | R90 | 193 | GCCCATTTTTATCUACTTCCATCTTGUCA |
| F91 | 98 | TGTAAAGAGACAGCCUTTCCTCUGA | R91 | 194 | AGTTCACAAAUCCATCAATGTTGCUC |
| F92 | 99 | ACACUCTUGAGGGCCACAAAG | R92 | 195 | TGTGATTGUAGGGTCTCCCTUGAT |
| F93 | 100 | CCGCTCCUTGTAGCCAAUGA | R93 | 196 | GGGTCUGACGGGTAGAGUGT |
| F94 | 101 | CCACUTUGGAACAGGACCAAC | R94 | 197 | TCAAAGTCGTCAUCCTTCAGTTCUC |
| F95 | 102 | CTTTCTTCCACCUTTCTCCAGCUA | R95 | 198 | CATCGCTGUAGAACGCACCAUA |
| F96 | 103 | TCAAGCCCUCCAACATCCUAGT | R96 | 199 | GGAAACTTCTGTUCATACCGACATGUAG |

TABLE B

| Primer | | Primer Sequence | Primer | | Primer Sequence |
|---|---|---|---|---|---|
| Name | SEQ ID | (target seq of Primer A) | Name | SEQ ID | (target seq of Primer B/C/D/E) |
| F97 | 200 | GAGGTCGTATUCGTCCACAAAAUGGT | R97 | 469 | GAGGCAGGAGACCCUGUAGGAG |
| F98 | 201 | GAAACTGCTTAGUAACTAGCAGAAGTGTUC | R98 | 470 | GAGGATAUATGCCAUACCCCAGCAAA |
| F99 | 202 | GACAAAGTTGUGTGTTGTAAGUGGAACA | R99 | 471 | GACCAAGAAAGGCUTGTGTCTACATTTUT |
| F100 | 203 | GAGCACCAATCUTTCTTCTGCCTTTUG | R100 | 472 | GACCAAAUCAAGAAACCTGTTUGAGAGAA |
| F101 | 204 | GAGGGAUCCCAAGGAAGAGAAGUGA | R101 | 473 | GACACTTCCCUTGTGGGAATGUCAA |
| F102 | 205 | GAGCTACTCTCCUGAACTCTCTCACUC | R102 | 474 | GATGCATCAGAACCCUCCTTGAAUC |
| F103 | 206 | GACTCCCAGUTGCAACGTUAGGT | R103 | 475 | GAGGUCCACGGAUCCAGAAACAAG |
| F104 | 207 | GATCACGUGTCCCCCTUCCA | R104 | 476 | GAGGTTCCTCCUCTCCTGGTCUC |
| F105 | 208 | GAACATTTGGCUGTGACTTCUAAGAAGAAA | R105 | 477 | GAGCTCACTAACUAACGTGAAAGCCTUAC |
| F106 | 209 | GAGATGAUCCAGATGTUAGGGCAGT | R106 | 478 | GAGGTTTTGCACAAGUTAGGTTTGTTTUG |
| F107 | 210 | GAGCCCAAATUGATTTCGATGATCTUCA | R107 | 479 | GAACCTTTATATCGTUACTCTGAATCTTATCTUCC |
| F108 | 211 | GAGCTCAAGAGUGAGCCACTUCT | R108 | 480 | GATGACTCTGTCUCCTCTTGTCTTCUC |
| F109 | 212 | GAATTAACACACAUCAGTGGAACTTCUGT | R109 | 481 | GAAAACGTTTTUCACCTTAGCATTTUGT |
| F110 | 213 | GAGCAAUCCAAAAGAAUAGCAGCCAAA | R110 | 482 | GACACCATCUCCATATCATUGAGACCAAAT |
| F111 | 214 | GACTTTGTGGAAUAGCCCATGAAGAGUA | R111 | 483 | GACGACAGACUACTTTGGTTCTCTTTUGT |
| F112 | 215 | GAACAGGAAGAGCACAGUCACTTUG | R112 | 484 | GACTCACUGACAAGCTCCUCGT |
| F113 | 216 | GACCATGCAGAGUGAAAGGATAUCCC | R113 | 485 | GAGCCTTTTCTTUTGCTTCCCTTGUT |
| F114 | 217 | GATGGAGCCGCUGACACCUA | R114 | 486 | GACAGGACCUGGCCCUGAC |
| F115 | 218 | GAGGTGUCTAGCCCAUGGGAGAA | R115 | 487 | GACCCAUCACACACCATAACUCCAC |
| F116 | 219 | GAGTCTGGUCCACATTGCTCUCA | R116 | 488 | GAGCTGUCCCCTCACCATUCAG |
| F117 | 220 | GAGCAAGAGUACACACTCCTCATTUGG | R117 | 489 | GATCACAACCCACUGAGGTATATGTAUAGG |
| F118 | 221 | GACTGGTTTCUGGTGGGACCATUA | R118 | 490 | GACAUGCACCGGAAAAGCGAUG |
| F119 | 222 | GACTCCCAGGCACUTGATGATACUC | R119 | 491 | GACATTTCTAGGUTACAGGCCUGGAT |
| F120 | 223 | GAGGGCTUGGTAACGTCCUGT | R120 | 492 | GACTCCAUGCCCCTCACUCA |
| F121 | 224 | GATGTGTCAAGGAGUTCGAAGATUCAC | R121 | 493 | GAATTGAAAATCUTCCTGCCTUCCCT |
| F122 | 225 | GAGUACAGCCAGTGTGUCCGA | R122 | 494 | GAGCAAAAGTGGUCCTCTCTGAAUCT |
| F123 | 226 | GACCATCCGGGCUTTACGCAAAUA | R123 | 495 | GAATATCAUCCAGCCTGTGTCTTUCC |
| F124 | 227 | GACCCCACUGAACCTCTCTTACATUT | R124 | 496 | GAGAGGGAAGGCAGGAUCTCUAAC |
| F125 | 228 | GACCCUAACAGCCATGCTTTCUC | R125 | 497 | GACCAGGCAAUGGAAAGGGTACAUA |
| F126 | 229 | GACGGCGAUGCTGAGAACCAAUA | R126 | 498 | GAGAAUAAAGAGGAGCAGGTUGAGGAA |
| F127 | 230 | GACCGACGUTGACCGCAUC | R127 | 499 | GAGGGCAAAUGAGCCTCUCAGT |
| F128 | 231 | GAAAATATTUCAGTGTCCGTUCACACACAA | R128 | 500 | GATCCAGATTGAUCTTGGGAGTGUAAAAA |
| F129 | 232 | GACCACACUGACGTGCCTCUC | R129 | 501 | GAGTCTTTGTGUTCCCGGACAUAGT |
| F130 | 233 | GAGCGCCACAGAGAAGUTGTUGA | R130 | 502 | GAGGGTCUGACGGGTAGAGUGT |
| F131 | 234 | GACCACAAAAUGGATCCAGACAACUGT | R131 | 503 | GAGCTTGCTCTGAUAGGAAAATGAGATCUA |
| F132 | 235 | GAACTGTTTCGTAUTTATAGCTGATTTGAUGGA | R132 | 504 | GACCTCTTCCUCAGGATTGCCTUT |
| F133 | 236 | GACCTCAUTGCCCUCAACACAGT | R133 | 505 | GATCAGTCCGGTUTTATTTGCATCATAGUT |
| F134 | 237 | GACACCACGUACCAGATGGAUGT | R134 | 506 | GACCCAAAGACUCTCCAAGATGGGAUA |
| F135 | 238 | GAAGACATGCAUGAACATTTTTTCUCCAC | R135 | 507 | GATCCAGACCAGGGUGTTGTTTUC |
| F136 | 239 | GATGUGGAGCCTCTUACACCCA | R136 | 508 | GAGTGCCAGGGACCUTACCTTAUAC |
| F137 | 240 | GAACGTCTTCCUTCTCTCTCTGUCA | R137 | 509 | GACTGAGGUTCAGAGCCAUGGA |
| F138 | 241 | GAGAATGTGAAAAUTCCAGTGGCCAUC | R138 | 510 | GAGUCATATCUCCCCAAACCCCAAT |
| F139 | 242 | GAGGGTGTGUGGTCTCCCAUAC | R139 | 511 | GAGCCATAGGGCAUAAGCTGTGUC |
| F140 | 243 | GAGGATGAGCUACCTGGAGGAUGT | R140 | 512 | GACCTTGGTCCUTCACCTAACCTUG |
| F141 | 244 | GAGGTCACTGUACACCTTACACAUGAA | R141 | 513 | GACCCTCUTTAGCCAUGGCAAGG |
| F142 | 245 | GACATCACUGTAAACCTUGCAGACAAAC | R142 | 514 | GATGGTCTCUCATTCTCCCAUCCC |
| F143 | 246 | GAGCTTCTTGGUCGTGTTCTTCAUT | R143 | 515 | GACTCCTCCUGTGATCTGCAAUCT |
| F144 | 247 | GAUGGAAGCCCAGCCATTUCTAAA | R144 | 516 | GAGATGAUGAAGATGATUGGGAAACACAAG |
| F145 | 248 | GAGCCCCUGAGCGTCAUCT | R145 | 517 | GAGGGCTGUGCGTCACTGUA |
| F146 | 249 | GAGAGCTGGUGGAGGCUGA | R146 | 518 | GAGGAGCCCAGGCCUTTCUT |
| F147 | 250 | GAGUGACCGAGGACAACGUGAT | R147 | 519 | GAGCGTCCUACTGGCAUGACC |
| F148 | 251 | GACTCUGGGAGATCTUCACGCT | R148 | 520 | GACCACUCACAGGTCGTGUGT |
| F149 | 252 | GAGGATTGCAGAUTGGGCCTUG | R149 | 521 | GAACATGATGGAUGTCACGTTCUCAAA |
| F150 | 253 | GAATAATCCAUTGCCTGTCUAAAGAACACT | R150 | 522 | GATGTTAACCUTGCAGAATGGUCGAT |
| F151 | 254 | GAGACTTGGUGTTGTTGAUGGCAAA | R151 | 523 | GACUGCAGGATTCCUACCGGAA |
| F152 | 255 | GACCAACAUGACTTACTTGAUCCCCAT | R152 | 524 | GAATCACCAAAUGGCACCAUACGA |
| F153 | 256 | GAACCCUGGCCTACCTGGUC | R153 | 525 | GAAGTTCAAGCUGAAGAAGATGUGGAA |
| F154 | 257 | GATGAAGCAGCAAGUATGAUGAGCAA | R154 | 526 | GACTGACACCUAGCTGTGATCCUG |
| F155 | 258 | GAGGCACGGTUGAATGTAAGGCTUA | R155 | 527 | GAACTGATATGGUAGACAGAGCCUAAACAT |
| F156 | 259 | GACCACACCCUGTTCACTCCTUT | R156 | 528 | GAGTCTCAGTCAUTAGAGCACTCUGG |
| F157 | 260 | GAAAGGTGATCUATTTTTCCCTTTCUCC | R157 | 529 | GATTTCATACUGACCAAAACUCAGCCT |
| F158 | 261 | GAGCTTTTTGCUAAAATGCATGTTUCCAA | R158 | 530 | GAGACACGGCUTTACCTCCAAUG |
| F159 | 262 | GACAAAGAATGGUCCTGCACCAGTAAUAT | R159 | 531 | GAAGGCCTGCUGAAAATGACTGAATAUAA |
| F160 | 263 | GATCCTCATGUACTGGTCCCTCAUT | R160 | 532 | GAGTAAAAGGUGCACTGTAATAAUCCAGACT |
| F161 | 264 | GACAGATCTGTATUTATTTCAGTGTTACTUACCT | R161 | 533 | GAGACTCTGAAGAUGTACCTATGGTCCUA |
| F162 | 265 | GACATGTCAACAUCGCTCTAATUCAGAGA | R162 | 534 | GAGCTTTUCAAAAGGCTUAAACACAGGAT |
| F163 | 266 | GATGTTACGCAGUGCTAACCAAGUT | R163 | 535 | GAGCAAACCACAAAAGUATACTCCAUGGT |
| F164 | 267 | GAGCTGAUTTTGGTCTUGCCAGAG | R164 | 536 | GATCTGACTTGGUGGTAAACTTTTGAGUT |
| F165 | 268 | GACCTCACCTCUATGGTGGGATCAUAT | R165 | 537 | GAGTTCTTGCUGGTGTGAAATGACUG |
| F166 | 269 | GATTCGCCTGUCCTCATGTATUGG | R166 | 538 | GACACCCCCAGGAUTCTUACAGAAAA |
| F167 | 270 | GAGCACUGGGACTTTGGTAATUCAC | R167 | 539 | GACATCTCTUGGAAACTCCCATCTUGA |
| F168 | 271 | GACAGTGAAAAACAAGCUCTCATGTCUG | R168 | 540 | GACCACATGUGTCCAGTGAAAAUCCT |
| F169 | 272 | GACAGTGTGUCCACCGTGAUCT | R169 | 541 | GAAGTGAAGGAGGAUGAGCCUGA |
| F170 | 273 | GATGGAATGCCAGAACUACAATCTTTUGAT | R170 | 542 | GAGTGGAAGATCCAAUCCATTTTTGTTGUC |
| F171 | 274 | GAGACGCAUTTCCACAGCUACAC | R171 | 543 | GAAGCATCAGCAUTTGACTTTACCTTAUCA |
| F172 | 275 | GAGCTTTGAAUCTTTGGCCAGUACCT | R172 | 544 | GACATAAGAGAGAAGGUTTGACTGCCAUA |
| F173 | 276 | GAGATGCAGCCAUTGACCTGTTUAC | R173 | 545 | GAAGAAAACCATUACTTGTCCATCGUCT |
| F174 | 277 | GAGGGATUAAAGCTGGCTAUGGCA | R174 | 546 | GACCTTGTUGGGACCTCAGAUGT |
| F175 | 278 | GAAGCAUACGCAGCCTGUACC | R175 | 547 | GAGTGGUAGCAGTGGAUGCAGAA |
| F176 | 279 | GAGCTUCCAGGAGCGATCGTUT | R176 | 548 | GAAGGCCCCAUACAATTTGAUGACA |
| F177 | 280 | GAAGCTCGTUCATCGGGACTUG | R177 | 549 | GACCATGGUGCACCUGGGAT |
| F178 | 281 | GACTGGTUACTGAAAGCUCAGGGAT | R178 | 550 | GAACTTTGCGUGGTGTAGATATGAUCAA |
| F179 | 282 | GAGGACTCTGUGAGTGGGATTTGTTUT | R179 | 551 | GAGTCTTCACUCACCTCGGAUGA |
| F180 | 283 | GACATCCCUGACTGTGAGAUCAAGAA | R180 | 552 | GACAGGUACGCCTCCAGAUGAG |
| F181 | 284 | GAATCAACCUGCTTGGTGTCUG | R181 | 553 | GAAACUCCCGCAGGTTUCCCT |
| F182 | 285 | GAACGAGGACCUGTGGGACUC | R182 | 554 | GAGTGCCTUGCCCTTTTTGUGG |
| F183 | 286 | GACATUCCCCAACAGCTGUGGT | R183 | 555 | GACCGGGAUGCCAGGAUACG |
| F184 | 287 | GACCTTCCUCCTGAAGGCCUGA | R184 | 556 | GAGGGCUGTACCTCCUCAGAGA |
| F185 | 288 | GAAAGTGCUTGTGCCCUGCAT | R185 | 557 | GAACAGGCUGCCCAAGGGCUA |
| F186 | 289 | GACCCCTCTUGGACCTTAGAUGC | R186 | 558 | GACAGUGATCAGAUGAGCAGCAG |
| F187 | 290 | GACGCAUGGAGAAGAAACTGCAUG | R187 | 559 | GAACGGUCTUGGAACCCAGAGA |
| F188 | 291 | GATCCCCTAUGTGCAAGTCCUAAAG | R188 | 560 | GAGCTATTGAUGTCTGCAGTCUGG |
| F189 | 292 | GACCACTGUGCAGAAGCTCUCC | R189 | 561 | GATUGACTUGCCGGAAGAGCCT |
| F190 | 293 | GACGCCGGCCUCGTGAGUC | R190 | 562 | GAAGACCUCCGAGTCACTCCUG |
| F191 | 294 | GACAAATGCUGAAAGCTGTACCAUACC | R191 | 563 | GAAAAAGACTCGGAUGATGTACCTAUGG |
| F192 | 295 | GATGAGGCAGUCTTTACTCACCUG | R192 | 564 | GATTCCTTTCTUCCCAGAGACATUGC |
| F193 | 296 | GAGCAAAGACUGGTTCTCACUCACC | R193 | 565 | GAACATCCCUCTCTGCTCUGCA |
| F194 | 297 | GACGAUCTGTTCUACACGGAACCC | R194 | 566 | GAGGCTGGTTAUTGAAACCTTGTTTUACAT |
| F195 | 298 | GACCAGACAAGCCUACAGTAGGAAUC | R195 | 567 | GACTACCCCCGUACCAAGUACAAAC |
| F196 | 299 | GACTCCACAGACCCUCTCCTUGC | R196 | 568 | GATCGUCGAAGCGGCUGAC |
| F197 | 300 | GAAGGGTGTCUCTCTGTGGCTTUA | R197 | 569 | GAGACTCTGUAGGCTGCAGTTCUC |
| F198 | 301 | GAGTTTCUGCAGATTGACTUGCACA | R198 | 570 | GACTTCTTCCUACCTGTTTCCCAUGAC |
| F199 | 302 | GATCAGGAAACAAAAAUTTGTGCTAUGCAA | R199 | 571 | GAGGACCCAUTAGAACCAACUCCAT |
| F200 | 303 | GAGCTGGAGGAGCUAGAGCTUGAT | R200 | 572 | GAGGCTTGUGGGAGACCTUGAAC |
| F201 | 304 | GAGGGCTGUCGTGGTAGACTUAGA | R201 | 573 | GACCTGGTAGTCUCAAGCAGATGTTAAUG |
| F202 | 305 | GAAAGACTTCUCAAATTGTTGCCATTUCAG | R202 | 574 | GAAACGGACATGAGUTTGTTTTCCTTCUA |
| F203 | 306 | GACGAGGAAGAAAAACAAUCCCACTUG | R203 | 575 | GACAGCCAACAAGAUTCTGAAGAACAUG |
| F204 | 307 | GAGAGATCCTTUCGAAGTCATCGTCUT | R204 | 576 | GATCCCTAGGUAGCTAACCCCUAC |
| F205 | 308 | GAGGGCAAUGTCAATTAGCUGGAAC | R205 | 577 | GAAAAACACGGCAUGTGAACATTCUG |
| F206 | 309 | GACACTGTGTTACUGCCATCGACTUAC | R206 | 578 | GAGTATTCAUCGAGATTUAGCAGCCAGA |
| F207 | 310 | GACTTTTACCCUCTTCAGCTCAGTTUCT | R207 | 579 | GAGAGAGAGGACUGACTATCGGACUG |
| F208 | 311 | GATCTCCTCCAACCUAATAGTGTATUCACA | R208 | 580 | GAGACTGUCAAGCAGAGAATGGGUAC |
| F209 | 312 | GATTTCGUAAGTGTTACUCAAGAAGCAGAA | R209 | 581 | GAGAATAGGATATTGUATCATACCAATTTCUCGAT |
| F210 | 313 | GAATGCCCCCAAGAAUCCTAGUAGAA | R210 | 582 | GAACGAAAATGUAAGAAGATTCATCTUGAAGAAG |
| F211 | 314 | GAAGAGATGATTGUTGAATTTTCCTTTUGGG | R211 | 583 | GAAAAGCCATTTTUCCAGATACTAGAGUGT |
| F212 | 315 | GACGGAACUCGAATCGCUACCCT | R212 | 584 | GAGGGUCCCCAAGACACCUACG |
| F213 | 316 | GACTCGATGCUGTTCCCAGGUAC | R213 | 585 | GACCGAGAACUGAGGGTGGUACA |
| F214 | 317 | GATCATACAGACACUTCATTTGGAGUACC | R214 | 586 | GATACTAGAACUCAAAACACTGGCTGUT |
| F215 | 318 | GAAAAAATAAAGCTUGGCTTCAAGTTGUCT | R215 | 587 | GAGTAAGTCTTCACUTTCAGATTTTAGTUGGG |
| F216 | 319 | GAACATTGTGACCUTAATTTTGTGATCTCTUG | R216 | 588 | GATGCTTCCTGGUCTTTAGGATTTCUT |
| F217 | 320 | GACCCCACTCAUGTTTAGCAGATGUA | R217 | 589 | GATTTTACTTCUGCTTGGTGGCAUG |
| F218 | 321 | GAGGACAGGUTTTGTTGTUGAGGAAG | R218 | 590 | GATTTTACCCTCAUGGCTTAGTAGCATTATUT |
| F219 | 322 | GAAGCAAGGTCAUAAATTATTCTCCATATTTUCCA | R219 | 591 | GAAAAAATATUCATCCAGCTUCAGGAAAAGG |
| F220 | 323 | GATCTTTTTACCTUATAGATGGGAAACAUGAGAG | R220 | 592 | GAATCAGTCUGGTGGATGGGUAACA |
| F221 | 324 | GAATGTGTCTTTCAUGAGAAAAACAAGATCAUT | R221 | 593 | GACTAATAATGAATAAUTGGGTATGAGGCUACAGT |
| F222 | 325 | GATAGTAGCTGAUCCACAGAAGTTCAGUA | R222 | 594 | GATGTGAGAGAGCAAUCAAGGAGUG |
| F223 | 326 | GAAAGACTCTGAAUACCACCATCAAGAATAAUAAA | R223 | 595 | GAGTCTGAGAGUAGAAGGCAGATTCTGUA |
| F224 | 327 | GAATCTACAGGCCAAUGGTTCCTUC | R224 | 596 | GAAACTTUGCGGAGATCUGAAAACCA |
| F225 | 328 | GAGCCAGTAGUCACAAAGATTTCTUACCA | R225 | 597 | GACTCTAAAGAAGGAAGUGAGAACTTCUCC |
| F226 | 329 | GAAGAAGAUTGGGUGGGCAGAC | R226 | 598 | GACTTTCTTCACUCAAAGTGCCTATTTUGAC |
| F227 | 330 | GAAACCACTGATACAUTTTTCTACTTTCCUGAA | R227 | 599 | GATTCTTTTGAGAACUGAGTGATTTAUGACCT |
| F228 | 331 | GAAGACTTCTTUGAGATATTTCCATAGCUCAC | R228 | 600 | GAAGAAGTUAGAAACAGAACTGTATGUAAGCAT |
| F229 | 332 | GACATTTTTGTTTATGUTATTCTCTCTACCUCAGC | R229 | 601 | GAGCTATACGAACTUAGAAGTGAGAAATAATCUT |
| F230 | 333 | GAAGTTATAGGTAAUCGATGCATATAGCTCAUCT | R230 | 602 | GATTTCTCCAGGUCCAAAATGAATAACTATTUGA |
| F231 | 334 | GAAATTGTTTGTAGGGUTGGTTATTAGTGACUAT | R231 | 603 | GAATCCAGGAUAGGAAGCACACAUG |
| F232 | 335 | GACCACTATGUAAGACAAAGGCUGGT | R232 | 604 | GATTTTATAACTAGATUTTCCTTCTCTCCATUCC |
| F233 | 336 | GAGTTTCTGUAGCCCATACTTTGGAUGA | R233 | 605 | GAAATTCATACAUTTTTCTCTAACUGCAAACAT |
| F234 | 337 | GACACTGUGAAGGCCCTTTCTTCUG | R234 | 606 | GAGCAGTTGUGAGATTATCTTTTCAUGGC |
| F235 | 338 | GAGCATAGGAGATAAUCATAGGAATCCCAAAUT | R235 | 607 | GATGTTTTTCTAAUGTGTTAAAGTTCATUGGAAC |
| F236 | 339 | GAAGTCACTGGAAUTGTTGGGCUAC | R236 | 608 | GACCATGACTGUCACAGTGACCUT |
| F237 | 340 | GACTUCCAGGAGCCGUAGAGTTT | R237 | 609 | GATTGUGGCCCAAACAAAGCUC |
| F238 | 341 | GAACTCTTCCTATTUTTGTAGTGACCTGTUT | R238 | 610 | GAGTGCTTGGAAAUGGAATGGTTTUAGAAT |
| F239 | 342 | GATTCCTGAUAAAGCACAGCTGTAGUG | R239 | 611 | GACTACTGUAACCAAGAGGTGACTUCAG |
| F240 | 343 | GATTGTUCAAGCAGCGAGUCC | R240 | 612 | GAAGCCGAUATCCCUGCAGAC |
| F241 | 344 | GACATGAACUACCTGGACCGCUT | R241 | 613 | GAGTCGGUGTAGAUGCACAGCT |
| F242 | 345 | GAGGTGCTGUCTGGGAAGAUGT | R242 | 614 | GATGCCCAAGGUACTGCAUGGT |
| F243 | 346 | GAAAAGAGGCAGUAGCATCTTCUCC | R243 | 615 | GACUCACGCCUAAACCAGAACC |
| F244 | 347 | GAAATCCTGGAGCUTTGGTGTCTAATUC | R244 | 616 | GATAGCTGGCUCCGCACCUT |
| F245 | 348 | GATGCAGAAGCGGUTTCTGUG | R245 | 617 | GACTGCUGGGCGCCGUAAC |
| F246 | 349 | GAGCCTCAGAGAUAAAGGCAAAGATUG | R246 | 618 | GAACCCACACAAGCGAAUCTCUG |
| F247 | 350 | GAACCATTATTTCTTUGTTTTGTTTTTCCTGUAT | R247 | 619 | GAACTTTGCUGCCTTAATGACATUCC |
| F248 | 351 | GATACCAACCAAGUTTCATUAACCACAGT | R248 | 620 | GAGGTACCUGAGATGGAGGAGUC |
| F249 | 352 | GACCATGTTGGUCACTTACTCAAAGATTUT | R249 | 621 | GATTGAGCCACUAAGCAGTAACCATUC |
| F250 | 353 | GAAATGGGCAAGGUATGGATGUGG | R250 | 622 | GACCCGAAGUTCTTCTGCAGUCC |
| F251 | 354 | GATGGCGCAUCAGATCCTAGTUT | R251 | 623 | GAGCCGAAACGAUCAAGGUGAGT |
| F252 | 355 | GATGTAACAACCUAAAGGGAATAGGAAGAAUG | R252 | 624 | GATCAGTAGAAAGAUGGTACCAAAAUGGGT |
| F253 | 356 | GAGGCACTGGUTCTCATTCCUG | R253 | 625 | GAGACCGAGCUCGGGTGUAT |
| F254 | 357 | GAATGACTCAAUACCAACCCCUCCA | R254 | 626 | GAGCCTTTGTGGUCATGGGAAAGTAUA |
| F255 | 358 | GACATAACCATGAUATTAATAGGACTCCUGCT | R255 | 627 | GAATTTGCCTGAAATUACACATAGAACTTTCUG |
| F256 | 359 | GATGAACGCAAAACCUGTTGAAGTUAAAA | R256 | 628 | GAGGGATGGGUGACTGAGAUGGT |
| F257 | 360 | GACTTAAAAATGTCAAUATCTGGCCTCAAAUACG | R257 | 629 | GAGTGGACAUGCGAAUGGAGGA |
| F258 | 361 | GAGGGAAGCAATUTGCTACACTTTAATTUAAAC | R258 | 630 | GATTTACTCTGACAGCUAAATGAACTCAAATGUA |
| F259 | 362 | GAACAGAAGCUTCTAATCCUCAACGT | R259 | 631 | GAAGGCTCAGAACACUTTACTGAATTTUG |
| F260 | 363 | GAAGTTGAUGCCAATTCACAAUCACCA | R260 | 632 | GATTACTTAGAAGAAAAUTGCTCCTTGUCAGA |
| F261 | 364 | GACTGAGGTCTATUCACTTTCTTTTCATCTTUG | R261 | 633 | GAGTGAAGGAAACCAUTCGTGAUAAAGC |
| F262 | 365 | GACTATTTGTTTCTUCCCCATGGAATTGUC | R262 | 634 | GAGAAAATUGGACCCAGTTCTCUGCT |
| F263 | 366 | GATAATCTTUGAACTGCCTGUGCACT | R263 | 635 | GAATCTTTCAACUGTAAAATTCACTGUGGGT |
| F264 | 367 | GAAAGCAAUGGCTTGGGAAGUAAGA | R264 | 636 | GACCATTCTCAUATCCTAGGTCUGCCT |
| F265 | 368 | GATCTAAAGGTTTTTCUGATTTCCTCATTAGGAUT | R265 | 637 | GAGCACUCCATTTUGGACAGCAA |
| F266 | 369 | GATCCAGTCATTTUGAGAAAGACAACTUACT | R266 | 638 | GAGTTTATTTTCTGGUTTCAATAGAACAAGTUGA |
| F267 | 370 | GATTCATTAATATTTUCAGATCACCAGTTGATUG | R267 | 639 | GATTTGAAAGGTAGAUTGCCATAATGTATCATUG |
| F268 | 371 | GAATTAACTGTACCUCCAACTTTCTTACTATAUGC | R268 | 640 | GAATGTGGAATCTUTGTTTAGTTTTACTCUGGT |
| F269 | 372 | GATTAAGAAACUAGAAACTGTTTAGACUGCCT | R269 | 641 | GAATGGTTTAGTCUGACACATATTTAACACUT |
| F270 | 373 | GATGGTTCTGUCGACTAAACUGC | R270 | 642 | GATTTTCAAGTTAUAGAAACATGTCATGTTGUCA |
| F271 | 374 | GAACGGACACUATGTCCTTAAGCUGA | R271 | 643 | GACTAGACTTUGAGACCTGCTAAATAATUAGATG |
| F272 | 375 | GATTGGCAUGGCTTCTCUAGCT | R272 | 644 | GACAGGUCCAAGUGAACCAGGGA |
| F273 | 376 | GACACCTTCUACCGCTCACUGC | R273 | 645 | GAAGGGCACUGACCCTGGUA |
| F274 | 377 | GAATTGACUCTGAATGUCGGCCAA | R274 | 646 | GATCTGCAGGAGGGUGCTCTUA |
| F275 | 378 | GACTCTGCCCCUAAGAAACCUGGA | R275 | 647 | GAGGGCAACUACACCTGCATUGT |
| F276 | 379 | GAATTACTCUAACTTTCGCAUGCACAC | R276 | 648 | GATGCCAAGACAGUGAAGTTCAAAUG |
| F277 | 380 | GAAGUGGGCAGCAGTTTCUGA | R277 | 649 | GAGACACCACCUACTTCTCCGUCA |
| F278 | 381 | GAAACCAACTGCUTGTATGCTTTCUGG | R278 | 650 | GAAAGACCAAAAGAGAAUGGAAAGTACUGAC |
| F279 | 382 | GAGCAGCCUTACCTGGTUGGA | R279 | 651 | GAAGAGUATCCATCUCCAGGAGACG |
| F280 | 383 | GATTACAGCUCGTTGGUGCAGT | R280 | 652 | GATGTGGCUCTCCGCCCAUT |
| F281 | 384 | GAATGGAAACCCUGACAGAGTCTTUG | R281 | 653 | GAGCTACACCATUAGCTTCACTGATTUT |
| F282 | 385 | GATGTTATGAGCUTAGCACCTUGCAG | R282 | 654 | GACTGGATGGUAAGAGGAGTTTCTTCAUC |
| F283 | 386 | GACCAGTTCUGCAGTTAGAGGTUG | R283 | 655 | GACCTTTCCCCUCCCCTACCUAG |
| F284 | 387 | GATTTGATTCTTAAUCACCTAAGGAUGGCT | R284 | 656 | GACAAAACAAAGUCAAAGAGAATTATGAAATGUG |
| F285 | 388 | GAATCCGTACCUTCCACCAATCUG | R285 | 657 | GACAGGGATTTUGGTTACTACTTTGCUAAGA |
| F286 | 389 | GATATAACAATGAAUGACCAAAAGGAAATUAACAA | R286 | 658 | GATTGTCTTCUGGACACGTTCUGAAA |
| F287 | 390 | GATAACTTTCCATAUGCAAACCTACTGGCUA | R287 | 659 | GATATTAAGCTTTCUTGGAAAATTCTCTTUCCCT |
| F288 | 391 | GACCUGGACGTCTUGGAAAAGGG | R288 | 660 | GATTCAGAAGTUAGGAAAGGAGUCCAG |
| F289 | 392 | GATCTGGGUCAAGGAUGGCACA | R289 | 661 | GACACCUGUCACCCGCACAC |
| F290 | 393 | GAGCCCACUTCCCATCUGGGT | R290 | 662 | GACAGTCAGUAACGCCAGUGAGT |
| F291 | 394 | GATCCCCGCUGCTGUGCAAC | R291 | 663 | GAGUCCCGTGGUGCAAAGGC |
| F292 | 395 | GACCCACATGUCCAGCACCUT | R292 | 664 | GAATGTACACTAGTTUCCGGAATAAACCTTUT |
| F293 | 396 | GAGCTGGUGAAACAGGTAGUGAGT | R293 | 665 | GACTUGACCCCUGCGAGCCA |
| F294 | 397 | GACTCCAGGUCCTTGTGUGAGC | R294 | 666 | GACCCACUCAAGCTCAGCTGUAA |
| F295 | 398 | GACCTCACGAACUGTGCTGAUGG | R295 | 667 | GACTGAGAATGGCUACCTCTCGATAUG |
| F296 | 399 | GAGGATTCGAGAAGUGACAGGCTAUG | R296 | 668 | GAGGCTGGAGUTGGTGTTATAGTUCAA |
| F297 | 400 | GAATTGGTAGCUGGTGATGTTCCUC | R297 | 669 | GACTCACACATCUTGAGCCCATTTTTAUC |
| F298 | 401 | GACAGCTAATUCATCTGGAGAUCAAACCC | R298 | 670 | GACTGAGAGGGUGTCACATACCAUG |
| F299 | 402 | GATGTCAGTTCCCUCCTTTTCTATTTTCUC | R299 | 671 | GATTCATACCGACAUGTAGGACCTUGT |
| F300 | 403 | GATGGGCACGGUAATGCUGCT | R300 | 672 | GAACTTCUCACACCGCTGTGUT |
| F301 | 404 | GACTCUGCGGTGGTUGGCAT | R301 | 673 | GACCTCACCUCCGTTTCCUGCA |
| F302 | 405 | GACTCCACCUCCAGGAACTTACUC | R302 | 674 | GATCUGGCCCCCTUAGGAGGA |
| F303 | 406 | GAGGGATCTTGUGAAATGTCATCTGACUC | R303 | 675 | GATCATCCTCUCCCCATAGAAAAGUCC |
| F304 | 407 | GATCAACCCTGTUTTTCTCCCTCTTATUG | R304 | 676 | GATCTCUGCCATCATTUCCGGAAAG |
| F305 | 408 | GACTGUACAGCATGAAGUGCAAGAAC | R305 | 677 | GATGCAAGGAAUGCGATGAAGUAGA |
| F306 | 409 | GACCATTAACAUGGCCTACCAGAGUT | R306 | 678 | GAGTCGCUAACACGTGTGTGTUC |
| F307 | 410 | GATTGCCUAGACAGCACCGTAAUG | R307 | 679 | GATGGCTAAACTUGACCTTTTTACTCUGC |
| F308 | 411 | GAGACGCAGUTTCTTCTTCTCAUCG | R308 | 680 | GATTCCTCAGCAUCGACCTUGC |
| F309 | 412 | GATATCGAGTGTGUGCATATGTGTATGTUG | R309 | 681 | GAAATCTATATACTUCCTTACCTGGGATUGGA |
| F310 | 413 | GAAGGGAAAAUGACAAAGAACAGCUCA | R310 | 682 | GAACATGCTGAGAUCAGCCAAATUC |
| F311 | 414 | GAGGCCTGCUTTTGGAGTCCUAT | R311 | 683 | GAGCAGUGAAAAGAGTCUCAAACACAA |
| F312 | 415 | GAGAAGAGCCUCCACCATCUCCA | R312 | 684 | GACCCACAGGCCUTCTUCGAG |
| F313 | 416 | GACACACAUGCCATCATTCUAGGAAG | R313 | 685 | GACTGGUATGAGAAACUGCACGAGT |
| F314 | 417 | GAGAGGTTTUCCAGCACTCTGACAUAT | R314 | 686 | GAAATACCAATCUATTGTGGGCTCUGG |
| F315 | 418 | GATCTTCTCTGUTTCAGGGCAUGAAC | R315 | 687 | GACCTCCTTCUGCATGGTATTCTTUCT |
| F316 | 419 | GAAGATTCUGCCGAACCAATGGAUC | R316 | 688 | GAATTAAAGCAGUGCTCATGATUGGG |
| F317 | 420 | GATATGACTUGTCACAATGUCACCACAT | R317 | 689 | GACGGGACUCGAGTGATGATUGG |
| F318 | 421 | GAGTGCCCTATUACCTCAATCATCCUG | R318 | 690 | GACTTCACCTTUAACACCTCCAGUCC |
| F319 | 422 | GATCAGTTACTACCUGAAAATGACACTTUGT | R319 | 691 | GACTCCTCTAGCUATCTTAATGACTUGGAC |
| F320 | 423 | GATAAAGACCTUCTTCCGTGTGUCCT | R320 | 692 | GACTGCTTTCATUCATAGGGAAATACAUAAGAAA |
| F321 | 424 | GATACATTTATTTUGAGAAACTTGAGAGAACUTCA | R321 | 693 | GATTCAATATTTTAAAUAGTCTGGCCUAAACGGT |
| F322 | 425 | GAAGATGGTGATAGAUCTTTAAGAGAATTGCUT | R322 | 694 | GATGATTTCCAGUATTAATTGGCAAUAAGAGAAT |
| F323 | 426 | GAAGCTTTTGATAAGAGUTAGGAAATCACTAGUC | R323 | 695 | GAATGAAAGCUAAAACATAAGATGAAUGGGAAAA |
| F324 | 427 | GAAGGAUAAAAACCAGCATTATTTATTUGAGCA | R324 | 696 | GAATTATTTCTTACCACUTTTCCTTTCTCCUGT |
| F325 | 428 | GACTGACCCAUAATCTTGCACCATTUACC | R325 | 697 | GAATTGTGAGATUAACAGCAGGGAUACC |
| F326 | 429 | GATTTGAAATGAAUGTTCACGACAAAUGC | R326 | 698 | GAGCTTCATTGTCTUGATAAAATTTATGGTATCUT |
| F327 | 430 | GAACAACCAAAACAAUACACACAGAGATTUT | R327 | 699 | GACCAGCTCTTUCATATCTTAACATTUAGCAACA |
| F328 | 431 | GAACGGAGGGUCATGTGTATATTAAGUAAG | R328 | 700 | GAGCCAAAACATTTUGTCCCTTTCTATAATTUG |
| F329 | 432 | GAGAATTAAGUGTGTACTACUCCCAAGAGAAAA | R329 | 701 | GATGGACTTCAAGUGATCACTTGUG |
| F330 | 433 | GATATAGGATGAGUAGCTCCAAATTAATGAAUGT | R330 | 702 | GAAGCCTGUGGTGCTTTTUGCG |
| F331 | 434 | GAGCTGTAGAAUAGTCAAGAGGAATUGCA | R331 | 703 | GAAAGTCAAACUACACTCAGAACCUGAAT |
| F332 | 435 | GACTCAGTGCUCTAAATCCAGAGCUG | R332 | 704 | GAGCAAAGGCCAAAGAUAAAATGCTTACUG |
| F333 | 436 | GAATGAAATATUGTCAACTCTCTUAGGCAAAAT | R333 | 705 | GAAAGCTACAGAAUGTGAACAGTCTTCTUAAA |
| F334 | 437 | GATACTTUGCAAAGCTGAATUAGACAGCA | R334 | 706 | GAGAGGTAGAUGCTGTAATTGCTGAUACAA |
| F335 | 438 | GAACATGAGCAUCACATTTTCCTUGG | R335 | 707 | GACAAACACCTCCUGATAAATTGGCTTUG |
| F336 | 439 | GATGCCTTATGAAUATATTCACGCUGACT | R336 | 708 | GACCCTACUCCAAGGAGCUCAGG |
| F337 | 440 | GAACCAGGUAAGCACCGAAGUC | R337 | 709 | GACCCAGTUACCATAACTACTCUGAGAAAA |
| F338 | 441 | GAACCAAGCCGCUGGTUCA | R338 | 710 | GACTTGCAGAGCUATCCCCUAAAGC |
| F339 | 442 | GAAGACCCCUTTAACTCAAGACUGC | R339 | 711 | GAGCTGCACCGAGUCGTAGUC |
| F340 | 443 | GAGCGAGGAUATCTGGAAGAAATUCGA | R340 | 712 | GAGTCGUTGTCUCCCCGAAGG |
| F341 | 444 | GAUTCUCCACGGCCGACCA | R341 | 713 | GAATACAGTCCUGGATGATGATGTTTTUGA |
| F342 | 445 | GAAAGTCCCUCAAAAATAGGAGGUGCT | R342 | 714 | GAGGACAAGAAAAGUGCAACTUCCCA |
| F343 | 446 | GAGACAGAUCAGCAACAACCGAAAAUG | R343 | 715 | GATTTCATTGUTTTCCAACUCCGGGAT |
| F344 | 447 | GATTTGTCCAGAGACCUTTCTAACGTAUT | R344 | 716 | GATTUCCACAGAAACAACAUCGATTTCTTC |
| F345 | 448 | GATTTCUGAAGAGGACTTGTUGCG | R345 | 717 | GATGCATTUGATCATGCATTUGAAACAAGT |
| F346 | 449 | GAGATTTTUCAGTTAATAATAUCCCCCGAGCT | R346 | 718 | GAAGTCTGUGCGCGCTUGC |
| F347 | 450 | GAAATCGCCUCCGGAUCCC | R347 | 719 | GAGTGCGCACGUCGCAAUC |
| F348 | 451 | GAGTCATTCCTTCUTTTTAAAATGGTGCTUAAGT | R348 | 720 | GAGGATGTAUACAAAAGGCGGATGUG |
| F349 | 452 | GAGGTCCCCCACCUCTCTTTUG | R349 | 721 | GAGCCAGAGAGUCCCTTUCACC |
| F350 | 453 | GACTCUCCAGGAAGGCTCACAUC | R350 | 722 | GATGCCACTCUTTGGGTTGAGUT |
| F351 | 454 | GATGGCATUGCCTTGTCCTUG | R351 | 723 | GATTTCAAACUGGAGGCTTAUCACCAA |
| F352 | 455 | GAGGCAGAAAACCAAAACAUTGGCTUA | R352 | 724 | GAATACAAGCATGAAAAUCAAAACATATCTTCUGC |
| F353 | 456 | GAAATTGTTCCTCAAGUTTGTTTAAGGACTUAAAA | R353 | 725 | GAGTAAATGGTAGCTUTTATCATAATCACCAGUC |
| F354 | 457 | GAAGTGGTATCAUCCCCATTTAATAGCUG | R354 | 726 | GATCCATTCAAGACUTTAGCAGGTGGUA |
| F355 | 458 | GAACAAATACAAAACUGTCCACATCTATGTUG | R355 | 727 | GAGTTACTCTCATGUGAGAACCATTTGAAUGA |
| F356 | 459 | GAAACAAACCATAGCUATAATGAAGAACTTGCUA | R356 | 728 | GAGTTTTTCTTATCUCTTAAAATGTTTCTGCUACA |
| F257 | 460 | GAAATAGTTGATCAUACTTTGTAACAGAAUCACA | R257 | 729 | GAAAGGUACAAGTUAAGGCACACAGAAG |
| F358 | 461 | GACTCATCTCCCTUTAATTTTGGCACATTAUT | R358 | 730 | GATGGATCTUGGCACAATGAUAACAGG |
| F359 | 462 | GAAACTATCTUCTTTGGACTTCUGAAGAGAC | R359 | 731 | GATGCTATAGTACCAGUACCTTTTAAGGTUCA |
| F360 | 463 | GAAGTAAATAATGGTTUCTCCTTCTCTTACTTUG | R360 | 732 | GACCGTAAGGUGGCCTACTTUGC |
| F361 | 464 | GATTCCTGGUGGCATTCAAUAAAGCA | R361 | 733 | GACAGCGTTTTCUTGTATTCCTGTATTUAGC |
| F362 | 465 | GAAGGAGCAACUTAGGGATCUGGT | R362 | 734 | GAGGAACTGTGAAUGAACTTGTAGGUG |
| F363 | 466 | GAACCCCTAAUCTGGTCAACCUG | R363 | 735 | GACCUGACCAGGGCGUCAAA |
| F364 | 467 | GAAGAAATAGAAAACUACAGGACGTTAUCCAG | R364 | 736 | GAAGTTCATCTUCGAAGCTCAAATTUCAG |
| F365 | 468 | GATCCGCTTTCUAAAATGTCAGTTGUC | R365 | 737 | GATACAACAAAAUGTTTGACTTCAUGCAGGT |

TABLE C

| Primer | | |
|---|---|---|
| Name | SEQ ID | Primer Sequence |
| F366 | 738 | AAAACTCAGTAUCAACAACTACCGGUAC |
| F367 | 739 | CTCAGAAAUGGAAAAAACCTGCAGUAAA |
| F368 | 740 | GTTCCCTCUGCGTGTTCTCAUAA |
| F369 | 741 | AAGAACCTGUGTGAAAGTATCUAGCAC |
| F370 | 742 | AUAAACCAAACCCAUGCAAAAGGAC |
| F371 | 743 | GCATTGAUGGAAGGAAGCAAAUACA |
| F372 | 744 | CCAGCTTCAUAGACAAAGGTTCUCT |
| F373 | 745 | GTGGTTTCTUCCATTGACCACAUC |
| F374 | 746 | CAAAUGGGCAGGACTCTUAGGT |
| F375 | 747 | GUGAGGAAACTUCTGCAGAGGT |
| F376 | 748 | GGAAGCAGGGAAGCUCTTCAUC |
| F377 | 749 | TGGTTUGAAGAACTTTCTUCAGAAGCT |
| F378 | 750 | AGGGAGACUGUGTGTAATATTTGCG |
| F379 | 751 | GCCAGTATUGAAGAATGTTGAAGAUCAAAA |
| F380 | 752 | GCCAAAAGGAAGUCTGTTUCCAC |
| F381 | 753 | CATGCCACACAUTCTCTTTTTACAUGT |
| F382 | 754 | GTAGAGUGCTACACTGUCCAACA |
| F383 | 755 | CTCTGAGAAAGAAUGAAATGGAGTUGGA |
| F384 | 756 | AAACAAATTTUCCAGCGCTTCUGA |
| F385 | 757 | AGCAATAAAAGTGUATAAATGCCTGTAUGC |
| F386 | 758 | TCAACAAGTTGACUAAATCTCGTACTTUCT |
| F387 | 759 | CATTCTTACAUAAAGGACACTGUGAAGG |
| F388 | 760 | CCCTTACAGAUGGAGTCTTTUGGC |
| F389 | 761 | AAAGACCTTTUGGTAACTCAGACUCAG |
| F390 | 762 | ACATTCACUGAAAATUGTAAAGCCTATAATTG |
| F391 | 763 | GGTTGTGCTTTTUAAATTTCAATTTTATTTTUGCT |
| F392 | 764 | GGATGUCACAACCGUGTGG |
| F393 | 765 | AGTGAAAACUAAAATGGAUCAAGCAGATG |
| F394 | 766 | AAACTAGTTTTUGCCAGTTTTTUAAAATAACCT |
| F395 | 767 | TTTTTACCCCCAGUGGTATGUGG |
| F396 | 768 | GAAAACACAAAUCAAAGAGAAGCUGCA |
| F397 | 769 | ATATTTAGUAGCCAGGACAGUAGAAGGA |
| F398 | 770 | AAATATTTCAGUGTCCGTUCACACACAA |
| F399 | 771 | GCAGAUGCAAGGTATTCTGUAAAGG |
| F400 | 772 | ACCTACATAAAACUCTTTCCAGAATGTUGT |
| F401 | 773 | CCCTTTCTGTUGAAGCTGTCAATUC |
| F402 | 774 | AGAUGGTATGTUGCCAACACGAG |
| F403 | 775 | GATGTTTCCGUCAAATCGTGUGG |
| F404 | 776 | GTAGAACTATCUGCAGACACCUCAAAC |
| F405 | 777 | CCAGAACCACCAUCTTTCAGTAATTUG |
| F406 | 778 | ATCATAAAATGTUGGAGCTAGGTCCUT |
| F407 | 779 | TATGATGGAAGGGUAGCTGTUAGAAGG |
| F408 | 780 | GGTTAAAATGTCACUCTGAGAGGAUAGC |
| F409 | 781 | GGAAATTTGUAAAATGTGCUCCCCAAA |
| F410 | 782 | AATTCCTTGTCACUCAGACCAACUC |
| F411 | 783 | ACTAAGGTGAUGTTCCTGAGAUGC |
| F412 | 784 | ACTTTCCUTAATGTCATTTUCAGCAAAACT |
| F413 | 785 | CAGTCTGAACUACTTCTTCATATTCTUGCT |
| F414 | 786 | CTAGTTCTGCUTGAATGTTTTCAUCACT |
| F415 | 787 | TGGAATGTTCTCAUTTCCCATTTCTCUT |
| F416 | 788 | GTTTCGTUGCCTCTGAACUGAGAT |
| F417 | 789 | CCTTGATTTTCTUCCTTTTGTTCACATUCA |
| F418 | 790 | TTTCTATGCTUGTTTCCCGACUGT |
| F419 | 791 | GAUGAAAGCTCCTUCACCACAGAA |
| F420 | 792 | CCTAGAGTGCUAACTTCCAGUAACG |
| F421 | 793 | CTTGGAAGGCUAGGATTGACAAATUCT |
| F422 | 794 | TTGTTACTCTTCUTGGCTCCAGTUG |
| F423 | 795 | TTAGGTGGGCUTAGATTTCTACUGACT |
| F424 | 796 | TGCTTATAGGTUCAGCTTTCGTTTUG |
| F425 | 797 | CCACTATGUAAGACAAAGGCUGGT |
| F426 | 798 | TCCGTTTGGTUAGTTCCCTGATTTAUC |
| F427 | 799 | GTATTATCTGUGGCTCAGTAACAAAUGC |
| F428 | 800 | TTAAAGCCTCAUGAGGATCACUGG |
| F429 | 801 | AGTTCATCACTUCTGGAAAACCACUC |
| F430 | 802 | GGGATCAGCATUCAGATCTACCTTTUT |
| F431 | 803 | TTCAGCCTTTTCUACATTCATTCTGUCT |
| F432 | 804 | TACCCTGAUACTTTTCTGGAUGCCT |
| F433 | 805 | GAATCCAAACUGATTTCATCCCUGGT |
| F434 | 806 | AGCTGCCUACCACAAATACAAATTAUG |
| F435 | 807 | CAGAGTTCUCACAGTTCCAAGGTUAG |
| F436 | 808 | GAAGAAGAAGAAAACAAAUGGTTTUACCAAG |
| F437 | 809 | ATCACCACGTCAUAGAAAGTAATTGUGC |
| F438 | 810 | CATTCAAACTTACTUGCAAAATATGTGGUC |
| F439 | 811 | GCATAGGAGATAAUCATAGGAATCCCAAAUT |
| F440 | 812 | AGTTGTAGTTGTUGAATTCAGTATCAUCCT |
| F441 | 813 | TGTGCCTTTCCUAAGGAATTTGCTAAUA |
| F442 | 814 | AAAAGATAAUGGAAAGGGAUGACACAGC |
| F443 | 815 | CTGTTAAGGCCCAGUTAGATCCUC |
| F444 | 816 | AGGCAGTTCUAGAAGAATGAAAACTCUT |
| F445 | 817 | TGTACCTAGCAUTCTGCCTCAUAC |
| F446 | 818 | TAGACCTTTTCCUCTGCCCTTAUCA |
| F447 | 819 | CACATUATTACAGTGGAUGGAGAAGACA |
| F448 | 820 | CTTCTTTGGGUGTTTTATGCTTGGUT |
| F449 | 821 | GCAGAGCTUTATGAAGCAGUGAAGA |
| F450 | 822 | TCTTAAATGGUCACAGGGTTATTUCAGT |
| F451 | 823 | TTCCATTGCATCUTTCTCATCTTTCUC |
| F452 | 824 | TTCACTUCAGCAAATTTTTAGAUCCAGAC |
| F453 | 825 | TGCCCCTTUCGTCTATTTGUCAG |
| F454 | 826 | GGAGATTTTTCTGUGTTTTCTGCTAGUC |
| F455 | 827 | TGACAUACTTTGCAAUGAAGCAGAAAA |
| F456 | 828 | GGATCCTGATAUGTCTTGGTCAAGTUC |
| F457 | 829 | GGCACCAAAUACGAAACACCCAUA |
| F458 | 830 | ATATCTGTCAGTGAAUCCACTAGGACUG |
| F459 | 831 | TGAAGAAGCAUCUGAAACTGTATTTCCT |
| F460 | 832 | GGACTACTACTATAUGTGCATTGAGAGTTUT |
| F461 | 833 | TGGCTTATAAAATATUAATGTGCTTCTGTTUT |
| F462 | 834 | GGTAAAAAUGCCTATTGGAUCCAAAGAG |
| F463 | 835 | AATCTACAAAAAGUAAGAACUAGCAAGACT |
| F464 | 836 | AAGTGACAAAATCUCCAAGGAAGTTGUA |
| F465 | 837 | GAATTCTTUGCCACGTATTTCUAGCC |
| F466 | 838 | GGCTTCTTCAUTTCAGGGTAUCAAAAA |
| F467 | 839 | AATACAUACTGTTTGCUCACAGAAGGAG |
| F468 | 840 | ACCGAAAGACCAAAAAUCAGAACTAATUAAC |
| F469 | 841 | TCACAGAAUGATTCUGAAGAACCAACT |
| F470 | 842 | ATTACCCCAGAAGCUGATTCTCUGT |
| F471 | 843 | TATATGATCATGAAAAUGCCAGCACUCT |
| F472 | 844 | TTCCCATGGAAAAGAAUCAAGATGTAUG |
| F473 | 845 | ACTGTCAATCCAGACUCTGAAGAACUT |
| F474 | 846 | CAGGUGAUAAACAAGCAACCCAAGT |
| F475 | 847 | TGGCATTAGAUAATCAAAAGAAACUGAGC |
| F476 | 848 | GAATCAGGAAGUCAGTTTGAATTTACUCAG |
| F477 | 849 | GCCTGTUGAAAAATGACTGUAACAAAAG |
| F478 | 850 | TGAAGATAACAAAUATACTGCUGCCAGT |
| F479 | 851 | AGGAGGGAAACACUCAGATUAAAGAAGA |
| F480 | 852 | TTTCAGACTGCAAGGGGAAAAATAUT |
| F481 | 853 | TCTTCTTACAACUCCCTATACATTCTCAUT |
| F482 | 854 | CCAGTTGGTACUGGAAATCAACTAGUG |
| F483 | 855 | AAAAGAGCAAGGUACTAGTGAAAUCACC |
| F484 | 856 | AAAAACCTTGTTUCTATTGAGACTGUGG |
| F485 | 857 | AATTCAGCCTUAGCTTTTTACACAAGUT |
| F486 | 858 | TGACAAAAAUCATCTCUCCGAAAAACAA |
| F487 | 859 | AATAATTTTGAGGUAGGGCCACCUG |
| F488 | 860 | TCATAACTCTCUAGATAATGATGAATGUAGCA |
| F489 | 861 | GTATAGGGAAGCUTCATAAGTCAGTCUC |
| F490 | 862 | AGAAGATAGUACCAAGCAAGTCTTTUCC |
| F491 | 863 | TAGTACAGCAAGUGGAAAGCAAGUT |
| F492 | 864 | CAGGCTTCACCUAAAAACGTAAAAAUG |
| F493 | 865 | ATGAAATAUTTCTTTTUAGGAGAACCCTCAAT |
| F494 | 866 | ATATATTUTCTCCCCATUGCAGCACAA |
| F495 | 867 | AGGACATCCAUTTTATCAAGTTTCUGCT |
| F496 | 868 | TGGCTCTGATGAUAGTAAAAATAAGATTAAUGA |
| F497 | 869 | GCTGTATACGUATGGCGTTTCUAAACAT |
| R366 | 870 | TCCCGTGGCUGGTAAATCTGAAAUA |
| R367 | 871 | CCAAAACATGAAUGTTCTCAACAAGUG |
| R368 | 872 | ATTCCTGCACUAATGTGTTCATUCT |
| R369 | 873 | GUCCAAAGCGAGCAAGAGAAUCC |
| R370 | 874 | AGTTCCAGUAGTCCTACTTUGACACT |
| R371 | 875 | AGAGCACGTUCTTCTGCTGTAUG |
| R372 | 876 | AGTTGAATATCTGTTUTTCAACAAGTACATTUT |
| R373 | 877 | GCCTGGCCUGAATGCCTUAAA |
| R374 | 878 | CAATTTCAACACAAGCUAAACTAGUAGGAT |
| R375 | 879 | TCAACAAAAGUGCCAGTAGUCATTTC |
| R376 | 880 | CTGTTTTUAGCAAAAGCGUCCAGA |
| R377 | 881 | AGTCAGCCCUTGCTCTTUGAAT |
| R378 | 882 | TTGGCCAUACAAAGTGAUAAAGGACTT |
| R379 | 883 | TTTGCAGGGUGAAGAGCTAGUC |
| R380 | 884 | TGTACAAAUGGGACTAACAGGUGGA |
| R381 | 885 | AGCATACCAAGTCUACTGAATAAACACTUT |
| R382 | 886 | CCTGGAGTCGAUTGATTAGAGCCUA |
| R383 | 887 | AATGTGTTATGUGGCTCCATTATUAGCT |
| R384 | 888 | GCATTTTTACCUACGATATTCCTCCAAUG |
| R385 | 889 | ACCAGTAAAAAUAAAGAACCAGGAGUGG |
| R386 | 890 | TTATAGAGGTTTUCTACTGTTGCUGCAT |
| R387 | 891 | GCAGTTGTGAGAUTATCTTTTCAUGGC |
| R388 | 892 | CATCATTCACCCUTGGCACAGUAA |
| R389 | 893 | AAAUATTTTCTAGGAATUGCGGGAGGA |
| R390 | 894 | CAGGUAAUCGGCTCTAAAGAAACATG |
| R391 | 895 | CAGAGAGATUCGAGGCAGAGUG |
| R392 | 896 | AGTAGUGGATTTUGCTTCTCTGATATAAACT |
| R393 | 897 | GCTCTUAGCCAAAATATUAGCATAAAAATCAG |
| R394 | 898 | AAAAAGCATUGTTTTTAATCAUACCTGACTT |
| R395 | 899 | GGTACAGAUTTGTAAATCUCAGGGCAA |
| R396 | 900 | GAGAUCACGGGUGACAGAGC |
| R397 | 901 | ACCTACCTGAUACCCCAGAUCCC |
| R398 | 902 | TCCAGATTGAUCTTGGGAGTGUAAAAA |
| R399 | 903 | GTGTGCTAGAGGUAACTCATGATAAUGG |
| R400 | 904 | GAAAGGGUCAACAAAAGAATGUCCAT |
| R401 | 905 | GAAAGTTCCCCAAUTGAAAGTUGCAG |
| R402 | 906 | AACTTTGTAATUCAACATTCATCGTTGUGT |
| R403 | 907 | TAGATGATAGGUGGTACATGCACAGUT |
| R404 | 908 | ACCUGAATTATCACTAUCAGAACAAAGCA |
| R405 | 909 | GAACAGUACCCGTTCCCTUGA |
| R406 | 910 | CTTGAGGACCUGCGAAAUCCAG |
| R407 | 911 | TGGAAAGCTTCUCAAAGTATTTCATTTUCT |
| R408 | 912 | GCAGCGTTTAUAGTCTGCTTTTACAUC |
| R409 | 913 | AACGGGCTUGGAAGAAAATAAUCAAG |
| R410 | 914 | TCTGCTAGCUTGTTTTCTUCACAGT |
| R411 | 915 | AACAATATACCTUCTCAGTCTACUAGGCAT |
| R412 | 916 | CAGATAACTUAGAACAGCCTAUGGGAAG |
| R413 | 917 | GGCCAAAATUGAATGCTATGCTUAGAT |
| R414 | 918 | AGCACAATUAGCCGTAATAACATUAGAGAA |
| R415 | 919 | TGGACTCATTACUCCAAATAAACAUGGA |
| R416 | 920 | GTCTAATATCAAGCCUGTACAGACAGUT |
| R417 | 921 | TGCAGAATACAUTCAAGGTTUCAAAGC |
| R418 | 922 | AATAAATGTGUGAGTCAGTGUGCAG |
| R419 | 923 | AAGCCTTCAUCCGGAGAGTGUA |
| R420 | 924 | TAATGCUGAAGACCCCAAAGATCUC |
| R421 | 925 | GCCAAAUGAACAGACAAGUAAAAGACA |
| R422 | 926 | GCAAATTGATAGUTGTTCTAGCAGUGAA |
| R423 | 927 | CAGCAGTAUAAGCAATATGGAACUCGAA |
| R424 | 928 | GGAGCAGAATGGUCAAGTGATGAAUA |
| R425 | 929 | TTTTATAACTAGATTTUCCTTCTCTCCATUCC |
| R426 | 930 | AGAGCGTCCCCUCACAAATAAAUT |
| R427 | 931 | GAAAGAGTTCACUCCAAATCAGUAGAGA |
| R428 | 932 | GGTTCTGAUGACTCACATGAUGGG |
| R429 | 933 | CCCTGTGUGAGAGAAAAGAATGGAAUAA |
| R430 | 934 | AGGCUGAATTCTGTAAUAAAAGCAAACA |
| R431 | 935 | AGGGTAGTTCUGTTTCAAACTUGCAT |
| R432 | 936 | TGTATATTTTCAGCUGCTTGTGAATTTUCT |
| R433 | 937 | GACAGTTCTGCAUACATGTAACTAGUGT |
| R434 | 938 | GCGGAUACAACCUCAAAAGACG |
| R435 | 939 | TGUCAAGTTTCTCTUCAGGAGGAAAAG |
| R436 | 940 | AAGGAAAATAACUCTCCTGAACATCUAAAAGA |
| R437 | 941 | TGTTGAAGAGCUATTGAAAATCATTTGUGC |
| R438 | 942 | ACAGCTCAAAGUTGAACTTATTCACUAAGA |
| R439 | 943 | ATGTTTTTCTAATGUGTTAAAGTTCATUGGA |
| R440 | 944 | GCCAGTTTCCAUATGATCCATCTAUAGT |
| R441 | 945 | AGAAACCTTAACCAUACTGCCGTATAUG |
| R442 | 946 | GCCACTTTTUGGGTATCTGCACUA |
| R443 | 947 | TTCAAGAGGUGTACAGGCAUCAG |
| R444 | 948 | GGTCAGGAAAGAAUCCAAGTTTGGTAUA |
| R445 | 949 | CCTCAGCTCCUAGACTTTCAGAAATAUG |
| R446 | 950 | AAACTCCATCUCAAACAAACAAACAAATUAAT |
| R447 | 951 | CCTCCTGAATTTUAGTGAATAAGGCTUCT |
| R448 | 952 | GCAAAGCACGAACUTGCUGT |
| R449 | 953 | GTGAUGGCCAGAGAGTCUAAAACAG |
| R450 | 954 | TGACATCCCTUGATAAACCTTGTUCC |
| R451 | 955 | TTTTTGTCGCUGCTAACTGTATGTUA |
| R452 | 956 | GCTCCAACTAAUCATAAGAGATTTUAAAAGAC |
| R453 | 957 | AAGTAAGAAGGCCUGATTTGGATUCT |
| R454 | 958 | GCTATTTCCTUGATACTGGACTGUCAAA |
| R455 | 959 | ATTCCTTGAGUTTACATTAACTUACCAGAAG |
| R456 | 960 | ATGACAATTATCAACCUCATCTGCTCUT |
| R457 | 961 | TAAATTGUTTTTCTCCTGTUGAACCAGACA |
| R458 | 962 | CCTGCTTATTTUTCTCACATTCTUCCG |
| R459 | 963 | GGTTUAGAGACTTTCUCAAAGGCTTAGAT |
| R460 | 964 | GTGTTTUCACTGTCTGUCACAGAAG |
| R461 | 965 | AAAACTATCTTCUTCAGAGGTATCUACAACT |
| R462 | 966 | GTGACGUACTGGGTTTTUAGCAAG |
| R463 | 967 | GGCTTCTGATTUGCTACATTTGAAUCT |
| R464 | 968 | AGGTCTTTTTCTGAAAUATTTTGGTCACAUG |
| R465 | 969 | AGATATTGCCUGCTTTACUGCAAGAA |
| R466 | 970 | TGTATTTCCAGUCCACTTUCAGAGG |
| R467 | 971 | TTGTTTTCTTTTUCAAAGTGGATATUAAACCT |
| R468 | 972 | CAGAAGGAATCGUCATCTATAAAACTATAUGT |
| R469 | 973 | CTGTAGTTTTTCCTUATTACATTTTGCTTCUT |
| R470 | 974 | TGGGATTGAAAGUCAGTATCACTGTAUT |
| R471 | 975 | GTTACCTTTGAGCUTGTCTGACATTTUG |
| R472 | 976 | TTGGATTACTCTUAGATTTGTGTTTTGGTUG |
| R473 | 977 | ATGGTAGAGTTCUTGAAAATGGGTUC |
| R474 | 978 | GTATTTTATCTATATUCAAGGAGATGTCCGAUT |
| R475 | 979 | GCCTTTTGGCUAGGTGTTAAATTAUGG |
| R476 | 980 | GTCTACCUGACCAATCGAUGGG |
| R477 | 981 | AGCTTTTUGCAGAGCTTCAGUAGA |
| R478 | 982 | GGCCAGATAATTUAAGACATATGTTGUGC |
| R479 | 983 | GCTCCGTTTUAGTAGCAGTTAACUGT |
| R480 | 984 | GTCTGTTTCCUCATAACTTAGAATGUCCAT |
| R481 | 985 | TCACTGTGCGAAGACUTTTATGTCUA |
| R482 | 986 | TTTCACTTTGUCCAAAGATTCCTTUGC |
| R483 | 987 | AGAATTCTGCAUTTCTTTACACTTUGGG |
| R484 | 988 | GGACTGATTUGTGTAACAAGTUGCAG |
| R485 | 989 | TCATACAAATAATUTCCTACATAATCUGCAGT |
| R486 | 990 | CAATACTGGCUCAATACCAGAAUCAAGT |
| R487 | 991 | AACCTGCCAUAATTTTCGTTUGGC |
| R488 | 992 | GAAGTTTCCAAACUAACATCACAAGGUG |
| R489 | 993 | ATTTCAGAAAACACUTGTCTTGCGUT |
| R490 | 994 | ACCACATTATAUGAAAAGCCTTTTUGGG |
| R491 | 995 | GGUTTCTCTTAUCAACACGAGGAAGT |
| R492 | 996 | CTGTCAGTTCAUCATCTTCCAUAAAAGC |
| R493 | 997 | TATACCAUACCTAUAGAGGGAGAACAGATAT |
| R494 | 998 | GCTTGAAGATTTUTCCAAAGTCAGAUGT |
| R495 | 999 | GTTTTGCTTTUGTCTGTTTTCCUCCAA |
| R496 | 1000 | AGGCAAAAATTCAUCACACAAATTGUCA |
| R497 | 1001 | TCATTGGAGGGUATGAGCCAUCC |

TABLE D

| Primer | | | Primer | | |
|---|---|---|---|---|---|
| Name | SEQ ID | Primer Sequence | Name | SEQ ID | Primer Sequence (target of PrimerC) |
| F498 | 1746 | GAGUGUGCGUGGCUCUCA | R498 | 1275 | UGCCAUCAUUCUUGAGGAGGAAG |
| F499 | 1002 | ACAACUGCAGCAAAGACUGGT | R499 | 1276 | UGCAAUCCCUGCCCCGGUT |
| F500 | 1003 | AGUUAAUUUUGGUUACAUCCCUCUCUGC | R500 | 1277 | GGAUUGCAGGCUCACCCCAAT |
| F501 | 1004 | AUCGAUCUGUUAGAAACCUCUCCAG | R501 | 1278 | CUGGAUUUCCUCAUGGAAGCC |
| F502 | 1005 | GGACUCUGUAGGCUGCAGT | R502 | 1279 | AAAUCCAGUUCGUCCUGUUCA |
| F503 | 1006 | UGAGGCAGUCUUUACUCACCUG | R503 | 1280 | GAAACUGCCUCUUGACCUGUCC |
| F504 | 1007 | ACAAGCAAAGUCUCUAUGGUGAUUAUGT | R504 | 1281 | AGGACAGUCAUGUUGCCAGUAUUAAAAT |
| F505 | 1008 | CAACUACCAUCCAGCAACAGAAAAT | R505 | 1282 | CUUCCAUGACUUUGGCAAUCUGG |
| F506 | 1009 | GACAGAUGAGAGAAAUGCACUUAGAAGA | R506 | 1283 | GAACAUGUCCUAUUUGAAUUUUCCGACUT |
| F507 | 1010 | AGGAAUGUGUUUCUCCAUACAGGUC | R507 | 1284 | GACACAAAGACUGGCUUACAUUUUGAT |
| F508 | 1011 | CUUCAAGCAGUGAGAAUACGUCCA | R508 | 1285 | AGGCUGACCACUUCUACUCUGT |
| F509 | 1012 | AGGGUCCAGGUUCUUCCAGA | R509 | 1286 | GCACUCAGGCUGGAUGAACAA |
| F510 | 1013 | GAUAGUUUUGAGAGUCGUUCGAUUGC | R510 | 1287 | UGUCCAGGGCUAUCUGGAAGAUC |
| F511 | 1014 | CUCCACCACCUCCUCAAACAG | R511 | 1288 | GCAGCAUUUACUGCAGCUUG |
| F512 | 1015 | AUCAGCCAGGCACAAAGC | R512 | 1289 | UGACAGAAGUACAUCUGCUAAACAUGA |
| F513 | 1016 | CAUCUUUGUCAUCAGCUGAAGAUGAAAT | R513 | 1290 | CUCACAGGAUCUUCAGCUGACC |
| F514 | 1017 | GCCUAAAGAAUCAAAUGAAAACCAAGAGA | R514 | 1291 | ACUUUGUUGGCAUGGCAGAAAT |
| F515 | 1018 | GUGACCCGGAGCACUUCC | R515 | 1292 | GCCGUGGUGCUGACCAT |
| F516 | 1019 | CCACAUUACAUACUUACCAUGCCACT | R516 | 1293 | GUGAUGAUUGGGAGAUUCCUGAUG |
| F517 | 1020 | AUGGGACCCACUCCAUCG | R517 | 1294 | GCUCUGAUAGGAAAAUGAGAUCUACUGUT |
| F518 | 1021 | CCCUUCUAAGGACCCCCUCUUC | R518 | 1295 | CUCCAGCAGGGCUUCGAT |
| F519 | 1022 | CUCUGCCGGGCUUUGAUCUT | R519 | 1296 | GGACUUUGCAACUUCAACAAAACUC |
| F520 | 1023 | UACUACCGCCUCACACGCT | R520 | 1297 | CUAGGUGUCUCCCCCUGUAAG |
| F521 | 1024 | UUCCCUCUCUCCUUCUGCCUC | R521 | 1298 | AGGUUCAGGCCUUGCACT |
| F522 | 1025 | CCAGCAGAAGACAAAAAGACAAACA | R522 | 1299 | CCAGCCCAGGAAGCAAAGAG |
| F523 | 1026 | GGAGAGGGAGGAGAGCUAACT | R523 | 1300 | UUAAAACUGGUCUCGCUCUCCC |
| F524 | 1027 | AGAAGCUGUGCAUUUACACCGA | R524 | 1301 | GAAAGCGGGAAUCGCAGAAA |
| F525 | 1028 | UUUUGCUGAUGCUAUGCUCUCCAC | R525 | 1302 | GGAAGACCUCUUCUUCGCACUT |
| F526 | 1029 | CUGAUCCGCAAGCAUGCUC | R526 | 1303 | CAAAAGAGCUCCCCCAUCUCC |
| F527 | 1030 | UGAGCUCGCUCACUUGUGAUG | R527 | 1304 | AGAAGAGACAUCUGGACUUAGCCAA |
| F528 | 1031 | CAGGAUCCAAAUUCGUUCUGUGC | R528 | 1305 | AUCAUCGACGGUGGGUACAUG |
| F529 | 1032 | UACUAACAACUCUGGUCUGGACCAT | R529 | 1306 | CGUUGGUCCUGACGGUACUG |
| F530 | 1033 | AUGCUAUUUGGACAAUAAACUCACCUUG | R530 | 1307 | CCAUUCUGAGGACUGCUGGUUUAUA |
| F531 | 1034 | CUCCUUACCUCAUACAGUGCAGAAA | R531 | 1308 | CCAGGACCAUCAUCCUACUGUAA |
| F532 | 1035 | CCCUGAGUGCAGCUUCGAUC | R532 | 1309 | GAGUGUUUGCUCCUCACUCUUC |
| F533 | 1036 | CGAUCAUGGAUGGCGGGUAC | R533 | 1310 | CUUCAGGUUUCCUUCUCUCAUGGUT |
| F534 | 1037 | UUUAUGAAUGGAGAGGCUGCUG | R534 | 1311 | GGAUGAGCUCACAGAGCUGC |
| F535 | 1038 | ACCUCUCACCCUUAUAAGUCUUCUGA | R535 | 1312 | UCAAAAAGCAUGCCCAGACCUUT |
| F536 | 1039 | ACUUGAGCUUCCCUAGGACCA | R536 | 1313 | ACUUUGAGACUUCUGCUUUGCUC |
| F537 | 1040 | GGGAGACAAUACGUGUCGGG | R537 | 1314 | AUCGAAAAACUGUGCAUCUACACC |
| F538 | 1041 | AAGACAGGUAGCGAUCCAGGUAG | R538 | 1315 | AGACCCAGCAGUGACUGT |
| F539 | 1042 | CUAGGUGCCCAUGUCCAUCUG | R539 | 1316 | ACAAGUAUAAUGAGCACCCCUUCT |
| F540 | 1043 | GAUGUCCAAUGUACCUGAGGCAA | R540 | 1317 | GUGGACACACCUGUAUUCCUGAG |
| F541 | 1044 | CAGCAGAAAGAGGACUCAGAAUAGAAAAUC | R541 | 1318 | CCGACCAGUUGGGCAAAAUC |
| F542 | 1045 | AGAGAUUCGCUUGUGUGGGUUAAA | R542 | 1319 | GCAGAAGUCUGUUUUCUUCAUGGUT |
| F543 | 1046 | ACCAGGGUUACCUUGAUCUCC | R543 | 1320 | AGGCCAUGUUGGGUUAAAGG |
| F544 | 1047 | ACUUCUCAAUUGCUACGGGCAAUC | R544 | 1321 | AGAAUCUACAGCUACCAGAUGGCA |
| F545 | 1048 | AGAUCUCGGUGAACGAUGCAAT | R545 | 1322 | CCUAGUUUCCAGUGCAUCUGUACC |
| F546 | 1049 | UAUGUGGACUGCAGAAGAACUUCG | R546 | 1323 | GGUCCCCAUCCAUUCUUCCUAUUC |
| F547 | 1050 | UGUGGUUUAUGAACAAGCGAUUUGG | R547 | 1324 | UGUGGAGUGUUGGCUGUAUCUUUG |
| F548 | 1051 | GCUCAUAUCGAGAGGUAGCCAUUC | R548 | 1325 | CUCUGUAAGCGACUUUUGGUGAUAG |
| F549 | 1052 | GCAGACGAGCUUGACAUCAGAAA | R549 | 1326 | GCCCAACCAAUUGAGAAGUUUGUAA |
| F550 | 1053 | AAGACUUCGGGUGCUCUGUAC | R550 | 1327 | UUAGUCAGGAGUCUAAGCCAACAG |
| F551 | 1054 | GGGACAGACUGUCAUUCAAAAUAGGA | R551 | 1328 | CUUGCCCGCAUCUAUAGUUUCCA |
| F552 | 1055 | UGAAGAAAGUCCAGACCUCGGA | R552 | 1329 | AACUUCCGUUUUGAGUGUUUACUGAUUT |
| F553 | 1056 | GUCGACUGCCUGAUAAGACAUGA | R553 | 1330 | UUACUUGGAUAAAGUUCCAGAGCCT |
| F554 | 1057 | UAGUUUGGUUUCUCUGUCUGUUCGUG | R554 | 1331 | GCUUAUUGCCACCUACUUAACCUCT |
| F555 | 1058 | AAGCCUCCAUCGCUACCCT | R555 | 1332 | CUUCAGCCCUGCAGGGAAA |
| F556 | 1059 | UCAGGCGCCAAGUAGGT | R556 | 1333 | GGCAAGUUCAACAUUAUUCCCUUUUGUA |
| F557 | 1060 | AAAGCGGCUGUUAGUCACUGG | R557 | 1334 | UCUUCCUCAGGAUUGCCUUUACC |
| F558 | 1061 | UAAAGAUCAUGUCUCGGCUCAAGGA | R558 | 1335 | CAUACAGAGAGGGUCAUCAGUGAUAC |
| F559 | 1062 | GUGCACAGGUUAUUCUGAUUUCCC | R559 | 1336 | GAAAGUCUCCCACAAAGUAACCC |
| F560 | 1063 | AGAAGGGCUAGGCCAAUUGAC | R560 | 1337 | AUAGUCAUAGCCGGGCCACA |
| F561 | 1064 | GUCAGCCUGAACAUAACAUCCUUG | R561 | 1338 | CCAGUUUAUUGUAUUUGCAUAGCACA |
| F562 | 1065 | GGGACCUCCGGUCAGAAAAC | R562 | 1339 | GGACCCAUUAGAACCAACUCCAUAAA |
| F563 | 1066 | CUCCCAACCAAGCUCUCUUGA | R563 | 1340 | UACCUUAUACACCGUGCCGAA |
| F564 | 1067 | CCCAGAAGGUGAGAAAGUUAAAAUUCC | R564 | 1341 | CCACACAGCAAAGCAGAAACUC |
| F565 | 1068 | AGGGCAUGAACUACUUGGAGG | R565 | 1342 | UUCUUUCUCUUCCGCACCCA |
| F566 | 1069 | GCCUCUCCCUCCCUCCAGGAA | R566 | 1343 | GUGAGGCAGAUGCCCAGCA |
| F567 | 1070 | UGCCUCACCUCCACCGT | R567 | 1344 | CCAAUAUUGUCUUUGUGUUCCCGGACA |
| F568 | 1071 | AAGUGUAAGAAGUGCGAAGGG | R568 | 1345 | UGUGUUCCUUUGGAGGUGGC |
| F569 | 1072 | GCCUUUUUAACUGGUAGAGAUUGGUG | R569 | 1346 | GAUCCAGAGGAGGAGUAUGUGUGA |
| F570 | 1073 | UCAUCACCUUCCUUUCAUGCUCUC | R570 | 1347 | UCUUCCUCCAUCUCAUAGCUGUCG |
| F571 | 1074 | UCCUACGUGGUGUGUGUCUGAA | R571 | 1348 | CGUCCUGUUUUCAGGCCAAG |
| F572 | 1075 | UGAUCAUCGAAUUCUCCAAAAUGGC | R572 | 1349 | AUUAGAGGGACUCUUCCCAAUGGA |
| F573 | 1076 | GAUGAGAUGUGGUACAAGCAUUCCA | R573 | 1350 | CCACGGUGGAAUUGUUGCUG |
| F574 | 1077 | CCCCUACAGCAUUGUUAAGAAAGUAUUT | R574 | 1351 | AUACCAGGCUAGUAUAGAUGCUUAGGG |
| F575 | 1078 | CUGGGACUAGCAUGCUGACC | R575 | 1352 | CAGACACCAACUCCCGGAAUC |
| F576 | 1079 | CUGCCUGUCUCUGGUUCUGT | R576 | 1353 | CAGAACUCUCUCCCCAGCAG |
| F577 | 1080 | ACUUGGAGUGAGUUUGGAUGGG | R577 | 1354 | CAGCUUCAUGUCUGUGCCG |
| F578 | 1081 | UCCUGAUCUCCUUAGACAACUACCUT | R578 | 1355 | UCACACCGCUGUGUUCCAUC |
| F579 | 1082 | UGUUCCUAUUUCAGCCCCACUC | R579 | 1356 | GUUGUGAGCGAUGAGCACGUA |
| F580 | 1083 | GGAAAGGGUCCUCUGAUCAUUGC | R580 | 1357 | AAAAUCGUGUCCUGGUAGCAGAG |
| F581 | 1084 | CGAGGGCCGGUAUACAUUCG | R581 | 1358 | CCCACCAAAAUGAGAAAACUGUGUT |
| F582 | 1085 | GAAUGUGAAAAUUCCAGUGGCCAT | R582 | 1359 | UGUCCUCCUAGCAGGAGAGG |
| F583 | 1086 | AUACCCUCUCAGCGUACCCUT | R583 | 1360 | CCGUGGAUGUCAGGCAGAUG |
| F584 | 1087 | AGCGCUUUGUGGUCAUCCA | R584 | 1361 | AUACUGGACUCAUCUCUCCUUCCC |
| F585 | 1088 | AAACUAGCCCUCAAUCCCUGAC | R585 | 1362 | AAAGACCACCCCCAAGACC |
| F586 | 1089 | CACAGUUGGAGGACUUCCUCUUC | R586 | 1363 | AUAACUCCACACAUCACUCUGGT |
| F587 | 1090 | CUACAUGGGUGCUUCCCAUUCC | R587 | 1364 | UUGACAUGGUUGGGACUCUUGAC |
| F588 | 1091 | GUCCUCGUGGCCAUGAAUGAA | R588 | 1365 | UGGCAAACUUCCCAUCGUAGAC |
| F589 | 1092 | CCCAAUCCCCACACCAAGUAUC | R589 | 1366 | GUUGAUCAUUGUUCCUUCCCCUCA |
| F590 | 1093 | AUGUUCCUCCCUCAUCUCUAAUGGT | R590 | 1367 | CCAUCUUGUCAGGAGGACAGG |
| F591 | 1094 | UGGACUCGAGCAACAUUGAUGG | R591 | 1368 | GGCAGGAUCUCUAACCCAUUGAG |
| F592 | 1095 | GCUGAAGUACCAGACCUGCUA | R592 | 1369 | CUCAGCAGGUAACUCACACUUG |
| F593 | 1096 | GGAUUUGACCCUCCAUGAUCAGG | R593 | 1370 | CUUCCCUGGGUGCUCCAT |
| F594 | 1097 | UCACUCUCUCUCUGCGCAUUC | R594 | 1371 | GUGGAUAUGGUCCUUCUCUUCC |
| F595 | 1098 | CAUGAAGUGCAAGAACGUGGT | R595 | 1372 | GGCUAGUGGGCGCAUGUAG |
| F596 | 1099 | GCGGAUCAGAGCCUCAAAC | R596 | 1373 | AUCAAAGUCCAGCACCAGCA |
| F597 | 1100 | ACUGUCCUGUUUUGAUAUCCCAGAUUUT | R597 | 1374 | GGGAAUUGCAUUCACACGUUAACA |
| F598 | 1101 | CUGUCUCAAUAUCCCAAACCCUAAG | R598 | 1375 | UUUGUUUUGUUUUUCUGUUUCUCCCUCUG |
| F599 | 1102 | UAUUAGUAUGCCCCUGCAACGUG | R599 | 1376 | GAGGGUUGUUAGUGGAGCAUAUGA |
| F600 | 1103 | CAACCCUCCUGCCAUCAUAUUGA | R600 | 1377 | UGAGACAGGCCAGUGUUUACAUG |
| F601 | 1104 | CAACCAUGACAAGAUUUUCCCUUACC | R601 | 1378 | GAGACUGGAGAAUGUAUACACACCUT |
| F602 | 1105 | UGCCUGUGGAGGAACUUUUCA | R602 | 1379 | CGACAUCUCCUCGGGCUT |
| F603 | 1106 | CUUCCUCUCGCCCAUCACA | R603 | 1380 | CGUAGAGCUCCGGGUGUC |
| F604 | 1107 | AGUGCCUCCUCUCCCAUCUT | R604 | 1381 | CUACCCAGGGCCACUGUUUT |
| F605 | 1108 | CACUCCUUGCUUCUCAGAUGAAACC | R605 | 1382 | GGGACAUUCACCACAUCGACUA |
| F606 | 1109 | CAGGUACUCCCGCAGGUUG | R606 | 1383 | UGGCCUCUUCUCCUGUGC |
| F607 | 1110 | CACGCAUACGGUUUGGUUUGG | R607 | 1384 | CUUCUUCUUCCCAUAGAUGCUCUCC |
| F608 | 1111 | CUAGAAGCUCUCUAUCCCACACCT | R608 | 1385 | GAGGCAUUAUUUGACCGGAUCUAC |
| F609 | 1112 | CAAGGAAUGCCUUCAAAAAGUUGGG | R609 | 1386 | CUGAGUAUGAGCUUCCCGAAGAC |
| F610 | 1113 | AGAUGAUGAUCUCCAGGUACAGG | R610 | 1387 | CCUGCUAACACCCUGUUCG |
| F611 | 1114 | CGGCACUGCAUGCAAUUUCUT | R611 | 1388 | CUGCCUGUCUCUCUUGGCUUT |
| F612 | 1115 | CCAUUUAUAGCUGAGUCUCCAUCCUG | R612 | 1389 | UAUGAACUUCCAGAGGACCCAAAAUG |
| F613 | 1116 | CCCAGUUGUGGGUACCUUUAGAT | R613 | 1390 | GGAAAAGAACGGCAGUAAAUACGG |
| F614 | 1117 | CUUUCAAACGAGUCAAGCAAGAAUGG | R614 | 1391 | AAUACGGGUCCAUCAAUCACACG |
| F615 | 1118 | ACCACACUUUCCAUAAUGAGGCT | R615 | 1392 | CAGUACUUGGUAUUCUGUGCUAGGA |
| F616 | 1119 | CUUUUCCAUCUUUUCUGUGUUGGUC | R616 | 1393 | GGAAGCUGUCCAUCAGUAUACAUUC |
| F617 | 1120 | CAGACAAAUCCCAAAACAAACCUGA | R617 | 1394 | GGCCCUCCUUCAGUUUAGUUGAG |
| F618 | 1121 | GUAGCUACAGGACUCAGAUACGUG | R618 | 1395 | GGUGGAGGCGAUAGUGGAUAG |
| F619 | 1122 | GUAUUUGGGCGAAUGCAGUUUUUC | R619 | 1396 | AGAUGGAGAUGAUGAAGAUGAUUGGG |
| F620 | 1123 | CCAGAGAAAAGAGAGUUACUCACACA | R620 | 1397 | GUCAAGUGGAUGGCUCCAGAAG |
| F621 | 1124 | ACUGUGUUACUGCCAUCGACUUAC | R621 | 1398 | CCAGAAAUGUUUUGGUAACAGAAAACAA |
| F622 | 1125 | GGUAUUCUCGGAGGUUGCCUUT | R622 | 1399 | AUUCUCUCUUUAGGGAGCUUCUCUUC |
| F623 | 1126 | CUUGGUCGUGUUCUUCAUUCGG | R623 | 1400 | UGGAAGAGAAAAGGAGAUUACAGCUUC |
| F624 | 1127 | ACCACUGUGGAGGCAUUUG | R624 | 1401 | AUUGGUCUCUCAUUCUCCCAUCC |
| F625 | 1128 | AGUGAAGAUCUCCCACAUUAACACC | R625 | 1402 | GUUUAGGUUUUGGCAACGUGGAT |
| F626 | 1129 | CUUGCCCAAAGCAACCUUCUC | R626 | 1403 | UCACCAGAUGCUAUGUGCUAAUCC |
| F627 | 1130 | AUUGGUUGCGGCCAUCUCT | R627 | 1404 | UCCUACCUGUGUCCACACC |
| F628 | 1131 | ACCAAUUUCAUAGGCGUGGC | R628 | 1405 | GGCAUGGGACAGAGUCGUT |
| F629 | 1132 | GCCUAUCGCUCUGCUCUCUC | R629 | 1406 | UUGUGCAAGGAGAGAACCUCUA |
| F630 | 1133 | UAACCCAGCGACGAACUUUCC | R630 | 1407 | CCUAUCCCAGAACUGGAGACAGAAA |
| F631 | 1134 | GCCCCUGAGCGUCAUCUG | R631 | 1408 | UGUACACCUUGCAGUGGAACT |
| F632 | 1135 | CUGGUGGAGGCUGACGA | R632 | 1409 | AGCCCAGGCCUUUCUUGG |
| F633 | 1136 | ACAACGUGAUGAAGAUCGCAGA | R633 | 1410 | ACUGGCAUGACCCCCAC |
| F634 | 1137 | GGGAGAUCUUCACGCUGGG | R634 | 1411 | UGCCACUCACAGGUCGT |
| F635 | 1138 | GUCUGAGGAGCCCGUGT | R635 | 1412 | GCAGAAACUCCCGCAGGT |
| F636 | 1139 | UCCUCGGAGCAGUGAGGG | R636 | 1413 | ACUCCAGAUACUGCAUGCCT |
| F637 | 1140 | AGCCUCUCCACGCUCCCUC | R637 | 1414 | ACUCCCGCAGGUUUCCC |
| F638 | 1141 | CUCACAUUGCCCCUGACAACAUA | R638 | 1415 | ACGGGAAAGUGGUGAAGAUAUGUG |
| F639 | 1142 | GUGUCCUUUCAGGAUGGUGGAUG | R639 | 1416 | AGAAACAUGAUGGAUGUCACGUUCUC |
| F640 | 1143 | GGUGACAUUUUCAAAGCAGUGUAUCC | R640 | 1417 | UGUUAACCUUGCAGAAUGGUCGAT |
| F641 | 1144 | GGGUAUUCGAUGAUCCCUGUGG | R641 | 1418 | AUGACUUGGACCGCGUAGC |
| F642 | 1145 | CCUCCCCACCAGCAUGUUT | R642 | 1419 | GCAUCCUACCGUUGAAGCACT |
| F643 | 1146 | GGCUUUGGUGAGAUCCAUUGAC | R643 | 1420 | CACCUGGAACUUGGUCUCAAAGAUT |
| F644 | 1147 | GCAUGUACUGGUCCCGCAT | R644 | 1421 | AUUCCUACCGGAAGCAGGT |
| F645 | 1148 | UGGUUCUGGAUCAGCUGGAUG | R645 | 1422 | AUGACGGAAUAUAAGCUGGUGGT |
| R646 | 1149 | UGCCAACAUGACUUACUUGAUCC | R646 | 1423 | AAAAUAUCCCCCGGCUUGUGAG |
| F647 | 1150 | GGACUAGGCGUGGGAUGUUUUT | R647 | 1424 | GAAGAAGAUGUGGAAAAGUCCCAAUG |
| F648 | 1151 | AGUGGAUCCCCUCUCCACC | R648 | 1425 | GUCCCUGGCUGGACCAA |
| F649 | 1152 | GAGGUUUUCCAGCACUCUGACAUAT | R649 | 1426 | CACACAUUGGAGCAUGCCAUUC |
| F650 | 1153 | CGGUUGAAUGUAAGGCUUACAACG | R650 | 1427 | AGCCUAAACAUCCCCUUAAAUUGGAUT |
| F651 | 1154 | GAACGGGAAGCCCUCAUGUC | R651 | 1428 | CGGCUUUACCUCCAAUGGUG |
| F652 | 1155 | CCUUACUCAUGGUCGGAUCACAAAG | R652 | 1429 | GCAGAGAAUGGGUACUCACGUUUC |
| F653 | 1156 | CCCUUUCUCCCCACAGAAAC | R653 | 1430 | UCAGCCUGUUUCUGGGAAACT |
| F654 | 1157 | GUAGAGCAAAUCCAUCCCCACA | R654 | 1431 | UGGAGAGAGAACAAAUAAAUGGUUACCUG |
| F655 | 1158 | UGUGCUUUUAGGGCCCACC | R655 | 1432 | GAUUCUUAUAAAGUGCAGCUUCUGCAT |
| F656 | 1159 | UCUGUUCAAUUUUGUUGAGCUUCUGAAUT | R656 | 1433 | CAGACGUCACUUUCAAACGUGUAT |
| F657 | 1160 | UCAGUGUUACUUACCUGUCUUGUCUUT | R657 | 1434 | CAGGCUCAGGACUUAGCAAGAA |
| F658 | 1161 | UGAAUUAGCUGUAUCGUCAAGGCA | R658 | 1435 | UAAGGCCUGCUGAAAAUGACUGAA |
| F659 | 1162 | UGUUUCUCCCUUCUCAGGAUUCCUA | R659 | 1436 | AGUCCUCAUGUACUGGUCCCT |
| F660 | 1163 | AAACCCGCAAUCCGGAAC | R660 | 1437 | CUGAUCUCGCCAUCGCUGUA |
| F661 | 1164 | CCCUCCAACAUCCUAGUCAACUC | R661 | 1438 | GUUCAUACCGACAUGUAGGACCUT |
| F662 | 1165 | GCUAGAGCUUGAUGAGCAGCAG | R662 | 1439 | UCAAAGUCGUCAUCCUUCAGUUC |
| F663 | 1166 | CCAUGGAGUCGAUGAGCUGG | R663 | 1440 | CCUCCAGAUGUGAAGCCCT |
| F664 | 1167 | GCCCAGCUCUGAGAUCCUUUC | R664 | 1441 | GCUGGAGGAGCUGGAACUT |
| F665 | 1168 | CAUUUCUGACAACUGAACUGCUCUC | R665 | 1442 | UAAACAGGAGCACGAGGAUGC |
| F666 | 1169 | UUACCAGCUUGUUCAUGUCUGGAUUC | R666 | 1443 | UAUUCAUCACGGCGCGCUT |
| F667 | 1170 | ACUGAGCUUGUUGGAAUAAGGAUGUT | R667 | 1444 | GAGUCCAGGAGAAAAUUCACAUGAGG |
| F668 | 1171 | UUGUAAGUGCCCGAAGUGUAAG | R668 | 1445 | ACAACCCACUGAGGUAUAUGUAUAGGUAUT |
| F669 | 1172 | UACGCAGUGCUAACCAAGUUCUUUC | R669 | 1446 | AGCACAGUGAAUUUUCUUGCCAUC |
| F670 | 1173 | CAGUCAAGGUUGCUGAUUUUGGUC | R670 | 1447 | GGUGGUAAACUUUUGAGUUUGCAGA |
| F671 | 1174 | UAUGGAUGUUGCCAAGCUGUAUUCUG | R671 | 1448 | GGGAAGGAGUGGUACAACAGAT |
| F672 | 1175 | GGUGGUCCUACCAUACAUGAAACAT | R672 | 1449 | ACAGCUAGUUUGCCAGUUAGUAAGC |
| F673 | 1176 | GCAAGCAAAAAGUUUGUCCACAGAG | R673 | 1450 | CACUUAAUUUGGAUUGUGGCACAGA |
| F674 | 1177 | ACAUCUCUCACCUCAUCUGUCCT | R674 | 1451 | CUCUUGUCAUCAGCUCCCAGA |
| F675 | 1178 | UCCCUGUAGUCCCGGAUGAG | R675 | 1452 | GCGCCAGCAUCCAGAGAUAC |
| F676 | 1179 | AAUUGUUGCCAUUUCAGGGUUUCUG | R676 | 1453 | GAGCGUGUGAUGCAGCUCUT |
| F677 | 1180 | CUCACCUAUCUCCCAGGCCUAAAAUA | R677 | 1454 | GUUUGACCGAAGAACCAAUUAUACCC |
| F678 | 1181 | ACAAACGAGAUGCCUCUUCCAG | R678 | 1455 | GAUGCUUCUCUCCUUCUUCUCUUGG |
| F679 | 1182 | GGCUGUCGUGGUAGACUUAGA | R679 | 1456 | UUCCCCAACCCACAUUUCCUUUAUAG |
| F680 | 1183 | CUGAGUGUAUCCUGGAGGUUGUUG | R680 | 1457 | CCAAAACCCUCCUGAUGUACACG |
| F681 | 1184 | GCUUGGUUCUGAUGUUUGUAGUGUAG | R681 | 1458 | GUCACAGCUCCAGUGUCUGUC |
| F682 | 1185 | UCCUUGUUGGUGUCCAUUUUCUUGT | R682 | 1459 | GAGAUCCAGGCUACCUGGUAUGAG |
| F683 | 1186 | ACAUGCCAUCAUUCUAGGAAGCUC | R683 | 1460 | AAGGACGACCCAGAGCUGAT |
| F684 | 1187 | CAGGACCCGCUUCUCUGAAAG | R684 | 1461 | AAAUUAAAAGGCAAGUGGACUUCGG |
| F685 | 1188 | AAGACCCCUUUAACUCAAGACUGC | R685 | 1462 | CUGUUGGUGAAGCUAACGUUGAG |
| F686 | 1189 | UGCUCCAUGAGGAGACACC | R686 | 1463 | AAAAUGGGAAAGGUAUCCAGCC |
| F687 | 1190 | AAUGUAACCUUGCUAAAGGAGUGAUUUCT | R687 | 1464 | CCACAGAAACAACAUCGAUUUCUUCC |
| F688 | 1191 | AACUGGCAAAUAUAUCAUUGAGCCAAAUC | R688 | 1465 | ACAGGGAUGGUGGUGGUT |
| F689 | 1192 | GGUGUGAAAUGACUGAGUACAAACUG | R689 | 1466 | UUCUGGAUUAGCUGGAUUGUCAGUG |
| F690 | 1193 | AUGGUGAAACCUGUUUGUUGGACAT | R690 | 1467 | GGCAAAUACACAGAGGAAGCCUT |
| F691 | 1194 | CCUGCUCAUGGUCUUUGAGUAUAUG | R691 | 1468 | AUGUCUAUAGGGAAGGGAAGACG |
| F692 | 1195 | GCCACACGCAACUGUCUAG | R692 | 1469 | GUCGGUGCUGUAGAUAUCCCT |
| F693 | 1196 | GACAAUCCUUGCUUACCUGAGGAAC | R693 | 1470 | ACAUUGUCAAGUUCUAUGGAGUGUGC |
| F694 | 1197 | GCUCGGGAUCCAUAUGUGGUAAT | R694 | 1471 | CUGGCUGAAGGUGGGUUUGAUT |
| F695 | 1198 | GGCCCUAUACUUAGGCCCUUUT | R695 | 1472 | AAGUCACACGGCCCUCC |
| F696 | 1199 | AACUCACGGUGGCUGCT | R696 | 1473 | UUGUUCUCAUUGGCUUCAAAGAUCUUUA |
| F697 | 1200 | UGUCCUGGUCAUUUAUAGAAACCGA | R697 | 1474 | UCUCUUGGAAACUCCCAUCUUGAG |
| F698 | 1201 | UCUCAUGUCUGAACUGAAGAUAAUGACT | R698 | 1475 | UGAGCCCACCUGACUUGG |
| F699 | 1202 | UUGGUAGCUCAGCUGGACUGAUAT | R699 | 1476 | ACAUGAGAGCUUGUUUUUCACUGG |
| F700 | 1203 | AUGAAGCAGGCUGAUACUACACAG | R700 | 1477 | AGAGUGAUCUCUGGAUGUCGGAAUA |
| F701 | 1204 | UUGUGAAGAUCUGUGACUUUGGC | R701 | 1478 | ACCAGUGAGGGAAGUGAGGAC |
| F702 | 1205 | CCUUUGGGUUAUAAAUAGUGCACUCAGA | R702 | 1479 | UAAGCAUCAGCAUUUGACUUUACCUUAT |
| F703 | 1206 | GGGAAGAAAAGUGUUUUGAAAUGUGUT | R703 | 1480 | CAAACAAGUUUAUAUUUCCCCAUGCCA |
| F704 | 1207 | UGGCUUUGAAUCUUUGGCCAGUA | R704 | 1481 | GAUUUGAUCCAGUAACACCAAUAGGGUT |
| F705 | 1208 | GUCGAGGCAAUGGAAAAGCUC | R705 | 1482 | AAACACAAACUAGAGUCACACACCUT |
| F706 | 1209 | AGAACAGCUCAAAGCAAUUUCUACA | R706 | 1483 | AGCACUUACCUGUGACUCCAUAG |
| F707 | 1210 | AGCAAGAGGCUUUGGAGUAUUUCAUG | R707 | 1484 | UUGUGUGGAAGAUCCAAUCCAUUUUUG |
| F708 | 1211 | UGUUCAUGCUGUGUAUGUAAUAGAAUGUT | R708 | 1485 | AACCAUAUCAAAUUCACACACUGGC |
| F709 | 1212 | CUGGAAUGCCAGAACUACAAUCUUUUGA | R709 | 1486 | CUCUUGCUCAGUUUUAUCUAAGGCUAG |
| F710 | 1213 | CUCAAGAAGCAGAAAGGGAAGAAUUUUT | R710 | 1487 | CAUACCAAUUUCUCGAUUGAGGAUCUUUUC |
| F711 | 1214 | UGACAGCCAUCAUCAAAGAGAUCG | R711 | 1488 | CCGCAGAAAUGGAUACAGGUC |
| F712 | 1215 | GGGAUUUCCUGCAGAAAGACUUGA | R712 | 1489 | AGAAAAUCAAAGCAUUCUUACCUUACUACA |
| F713 | 1216 | AAGGCACAAGAGGCCCUAG | R713 | 1490 | UCCAGGAAGAGGAAAGGAAAAACAT |
| F714 | 1217 | ACCAAUGGCUAAGUGAAGAUGACAAT | R714 | 1491 | AUUUGCCCCGAUGUAAUAAAUAUGCAC |
| F715 | 1218 | AGGUUAUCUUUUUACCACAGUUGCAC | R715 | 1492 | GUCAAGAUCUUCACAAAAGGGUUUGA |
| F716 | 1219 | UUUUCUGUCCACCAGGGAGUAACUA | R716 | 1493 | GCCACUGGUCUAUAAUCCAGAUGA |
| F717 | 1220 | GACAAGUUCAUGUACUUUGAGUUCCC | R717 | 1494 | GCAUCUUGUUCUGUUUGUGGAAGAA |
| F718 | 1221 | AGCAAAUAAAGACAAAGCCAACCG | R718 | 1495 | UCAACAACCCCCACAAAAUGUUT |
| F719 | 1222 | AGUUUAAGAUGAGUCAUAUUUGUGGGUUUT | R719 | 1496 | UGGAUUUGACGGCUCCUCUAC |
| F720 | 1223 | CUGACCAUGUGGACAUUAGGUGUG | R720 | 1497 | UUAACACCUCCAGUCCCUCAUCUG |
| F721 | 1224 | CCUUCCCUCGGGAAAAACUGAC | R721 | 1498 | UAAGAUGUCCACUGCUGUUCCUUCAUA |
| F722 | 1225 | GUUUGGUUUUGUAGGUCUUGUGGAUG | R722 | 1499 | CUUCAGCCAAGGCAGCAAUG |
| F723 | 1226 | GAGGUGGCCUGAUCUUCACAA | R723 | 1500 | GAUAUGGAUUCACACAGACACUAUCACA |
| F724 | 1227 | CGCUUAUGCAUACUCAGGAUGAGUT | R724 | 1501 | CAAGGUGUUUCUUUGAUGCUCUGT |
| F725 | 1228 | UAAGGUUCCUUCAAGCUGCCCUA | R725 | 1502 | CCUGUGGACAUUGGAGAGUUGAC |
| F726 | 1229 | CAUGGGAGGAUGUUCUUUCCCAUUT | R726 | 1503 | GAACCUUAAAUGUCUCUCCUACCUGA |
| F727 | 1230 | UUUUCUUCCUAAGGUUGCACAUAGG | R727 | 1504 | AAGGCACCUGACCCAAACA |
| F728 | 1231 | AUUUUUGGCUUCCUGGCCUUT | R728 | 1505 | GCACAUAGUCCCGGAAGCUG |
| F729 | 1232 | GGAAAGCCUCACCUGUCUACG | R729 | 1506 | UUCUUGAUCUCACAGUCAGGGAUG |
| F730 | 1233 | UCAAGAAUCGCCCGAGCC | R730 | 1507 | AUGAGCAGCGUGGCCUT |
| F731 | 1234 | UUGGUUCGGACAGACAACCC | R731 | 1508 | UAGCUGUGCAUGUCCUGGUG |
| F732 | 1235 | CUCUGCACAGCUCCAAUGAGAC | R732 | 1509 | UAGGUGAGGACCACAAACCAAAC |
| F733 | 1236 | GCUACAAGAACUACCGAUACCGT | R733 | 1510 | UGGUCUUCACUCACCUCGGAT |
| F734 | 1237 | CUCGGAGAGGAGCCAUACUG | R734 | 1511 | UUCCUCCAGAAGCUUGAACUCT |
| F735 | 1238 | UAUAAUGACAGUUAACCCUGCCAGGA | R735 | 1512 | CCCAAGCCUGGGACCUCUAUUAT |
| F736 | 1239 | AGGAAGAGCACAGUCACUUUGA | R736 | 1513 | CAUGCUGGACCUUCUGCAC |
| F737 | 1240 | CAGUGGAGCGAAUUCCUUUGGA | R737 | 1514 | AGACUGCUAAGGCAUAGGAAUUUUCG |
| F738 | 1241 | UUGGGUCGUUGGGCAUUCC | R738 | 1515 | UUUGACUCUGUCUCCUCUUGUCUUCT |
| F739 | 1242 | CAGUUCACAGUGCAGCGAAAA | R739 | 1516 | GAGAUGAAGCAAACAACAGUGGAG |
| F740 | 1243 | AAAUAUCUACACACAGGUCUACAAGGUC | R740 | 1517 | AUUUCAUGCAAACUAGAUAACUACCUGUAA |
| F741 | 1244 | CAUCCGGGCUUUACGCAAAUAA | R741 | 1518 | UGGAGUUUGUCUGCUGAAUGAACC |
| F742 | 1245 | GCCUCCUUCAGGAAUUCAAUCUUCT | R742 | 1519 | AGCUCACAGAAAUGUCUGCUAUACUG |
| F743 | 1246 | AUGAGUUCUGGGCACUGGG | R743 | 1520 | AUGAGGAGUGUGUACUCUUGCAUC |
| F744 | 1247 | GAUGCAAACUCUUGCACAAAUGCT | R744 | 1521 | GCCAAGAGUUACGGGAUUCCAT |
| F745 | 1248 | GAACCCCGAGGGCAAAUACAG | R745 | 1522 | AGGAUGCCUGACCAGUUAGAGG |
| F746 | 1249 | CAGUUCGUGGGCUUGUUUUGUAUC | R746 | 1523 | AAAAGACUCGGAUGAUGUACCUAUGG |
| F747 | 1250 | UUAAAGCUGGCUAUGGCACCUG | R747 | 1524 | CACUCACCCUGGAUGUCUUCG |
| F748 | 1251 | CAUCUCUCACCAUCCCAAGG | R748 | 1525 | CACCGUAGCUCCAGACAUCA |
| F749 | 1252 | AUACGCAGCCUGUACCCA | R749 | 1526 | AAGGAGAAGAGGACAGCGG |
| F750 | 1253 | CACCUCUCUCAAGAGUUUGGAUGG | R750 | 1527 | CCUGCACUUCUAGGCACUUACUAA |
| F751 | 1254 | AGAUUGCGAGAGAGCUGCAT | R751 | 1528 | GGCACUUGCACAGAGAUGAT |
| F752 | 1255 | CUGUGCUGCAUUUCAGAGAACG | R752 | 1529 | AUUUGAUGACAUGUGGGUGGUUG |
| F753 | 1256 | AAGACCCAAGCUGCCUGAC | R753 | 1530 | GGAGCCGUAUUUGGCGT |
| F754 | 1257 | GCUAUUUUUCCUCACAGCUCGUUC | R754 | 1531 | CCUCUUCACGUAGGAAUCCUCUUC |
| F755 | 1258 | CUCCUUCCUAGAGAGUUAGAGUAACUUC | R755 | 1532 | AUCACUUUGCGUGGUGUAGAUAUGAT |
| F756 | 1259 | GAGCCUGUUUUGUGUCUACUGUUCUA | R756 | 1533 | AGGACUCUGAAGAUGUACCUAUGGT |
| F757 | 1260 | CUCUUGCAGCAGCCAGACT | R757 | 1534 | ACAGUUUCCAUAGGUCUGAAAAUGUUT |
| F758 | 1261 | CCAUGGGACUGACUUUCUGC | R758 | 1535 | AGCCCAACCCUUGUCCUUAC |
| F759 | 1262 | GCUGAGGACCUGGUCCUCT | R759 | 1536 | GGGACAGCAUCAAAUCAUCCAUUG |
| F760 | 1263 | CUGCACCAGCAGCUCCUA | R760 | 1537 | CCAGACGGAAACCGUAGCUG |
| F761 | 1264 | CCCGGACGAUAUUGAACAAUGGT | R761 | 1538 | GGAGCAGCCUCUGGCAUT |
| F762 | 1265 | AGCCUCACCACGAGCUG | R762 | 1539 | GGCAAGGAAAGGUGAUAAAAGUGAAUCT |
| F763 | 1266 | CACCUUUCCUUGCCUCUUUCCUA | R763 | 1540 | ACUUGAUAAGAGGUCCCAAGACUUAGT |
| F764 | 1267 | CUCAAGGAUGCCCAGGCT | R764 | 1541 | CCUAUGGCUUUCCAACCUAGGA |
| F765 | 1268 | CCUCCCUGCUUCUGUCUCCUA | R765 | 1542 | CCCUUCUGUCUUGAACAUGAGUUUT |
| F766 | 1269 | CCAGUUGCAAACCAGACCUC | R766 | 1543 | UGUGGAGUAUUUGGAUGACAGAAACA |
| F767 | 1270 | ACUCCACACGCAAAUUUCCUUC | R767 | 1544 | AGGCCUCUGAUUCCUCACUGAT |
| F768 | 1271 | AGGGUGGCAAGUGGCUC | R768 | 1545 | CCUAGGUUGGCUCUGACUGT |
| F769 | 1272 | GAGGCUCCCCUUUCUUGC | R769 | 1546 | UGCCUCUUGCUUCUCUUUUCCT |
| F770 | 1273 | CUGUGACUGCUUGUAGAUGGC | R770 | 1547 | UUCCUACAGUACUCCCCUGC |
| F771 | 1274 | CUGUCGUCUCUCCAGCCC | R771 | 1548 | AGUCACAGCACAUGACGGA |
| F772 | 1549 | GGAGGAGGCGAUGGCUACUA | R772a | 1574 | CUGCAGUUAGAGGUUGGUGACA |
| | | | R772b | 1575 | CCCGCCAAGCACGUAUACU |
| F773 | 1550 | GGAGACCUACAAACUGAAGUGCAA | R773a | 1576 | CCGGAAGAGGAGUAGCUGAC |
| | | | R773b | 1577 | CUCCUAGAGUUUUUCCAAGAACCAAGU |
| F774 | 1551 | CCAUGCAGAAUGCCACCAAGUA | R774 | 1578 | AU UUGCAGCUACUACUCUGAACUGAA |
| F775 | 1552 | CAGGCACUCCUUGGAGCAA | R775 | 1579 | UCAGUGGGAUUGUAACAACCAGAAAU |
| F776 | 1553 | CUGUUUGAAAUGAGCAGGCACU | R776 | 1580 | GCACUGUCACCCCUUCCUUG |
| F777 | 1554 | ACUGGAGGACCCGUCUUCU | | | |
| F778 | 1555 | AGACCUUAAGGGAACAGCUCUCAU | | | |
| F779 | 1556 | GUGGAGUCAUGCUUAUAUGGAGCAAA | R779 | 1581 | GCUCCAUCUGCAUGGCUUG |
| F780 | 1557 | GACAGAAAAAUAAUUCUGUGGGAUCAU | | | |
| F781 | 1558 | UCCUGAAAGAGAAAUAGAGGUUCCUGAU | | | |
| F782 | 1559 | GGTGGCCATAGGAACGCA | | | |
| F783 | 1560 | UGGAUGCAGAAACCAGAGAUCUAGU | | | |
| F784 | 1561 | CUGGUCCCCAGACAACAAGUAU | | | |
| F785 | 1562 | GAAGAUCAUGUGGCCUCAGUGAA | R780 | 1582 | GGGUUGUAGUCGGUCAUGAUGG |
| F786 | 1563 | GUCGAAAAUACCUUCAACACCCAAAUU | R781 | 1583 | CCUGGCCCUUGAAGCACUA |
| | | | R782 | 1584 | ACCCCAUCUUCCCCAUCCAU |
| F787 | 1564 | CCAAAACUGCAGACAAGCAUAAAGAUG | R786 | 1585 | CUACCUCACAGUGACUGCAGUUUA |
| | | | R787 | 1586 | AGAGAGGAUCAGCGAGAGUGG |
| F788 | 1565 | CAGGCAGAAGUUGAUCGACUCU | R788 | 1587 | GUCUCGUUGCCCAAAUUGAU |
| F789 | 1566 | AAAGAAGAGUGCACAAAUGUUAGAGGA | R789 | 1588 | AGUGUUUUCAUUCGAUUCCUGUCUUCU |
| F790 | 1567 | CCAGCUUCCUAUAACUUGGACGAU | | | |
| F791 | 1568 | GAACCACAUCAUGGUCUCUGUCU | R791 | 1589 | GGUGAUGCCGUGGUUGAUGU |
| F792 | 1569 | UCAUCGGGAAGACCUGGCUUA | R792 | 1590 | AGUUCUCGCUUCAGCACGAU |
| F793 | 1570 | GCUGCAGGACUAUGAGGAGAAGA | | | |
| F794 | 1571 | CUCCCAGAGACCAACGUUCA | R794 | 1591 | UGGCCAAGCAAUCUGCGUAU |
| F795 | 1572 | GGACCUGGACCGUGUCCUUA | R795 | 1592 | UGCCAGGAUCAUAGCGUUUACAG |
| F796 | 1573 | GGACCUGGACCGUGUCCUUA | R796a | 1593 | CUGGAGCAGGUCCACUAUAGGU |
| | | | R796b | 1594 | UCCUCACACCUGCUCCUCA |
| | | | R796c | 1595 | GCUGAUGGGUGGGCACUG |
| | | | R796d | 1596 | GGUCUACCAGGACUGUCCCU |
| F797 | 1760 | CCCUUCGUAGACAUAUAGCUGUUCUC | R797 | 1597 | GGAAGGCAGGAAGAUUUUCAAUCUC |
| F798 | 1761 | UGGUGCUAGUUGCAAAGACACAA | R798 | 1598 | CGUUUAUAAGCACUGUCACCCCUU |
| F799 | 1762 | AGCGACGCCAUUGCUCAU | | | |
| F800 | 1763 | CCUCAACCAUUUCCGGCAAAU | | | |
| F801 | 1764 | CCAGCUCCCUGCGAAGAG | R801 | 1599 | AGGAUGAUGGCACUGAACUCC |
| F802 | 1765 | AAUCCCUGCAGUAGAUACGAAGACUA | R802 | 1600 | CACGUUAGUUAGUGAGCCAGGUAAU |
| F803 | 1766 | AGACCUUGCAGAAAUAGGAAUUGCU | R803 | 1601 | CUCAGGGCUCUGCAGCUCC |
| F804 | | | R804 | 1602 | CCUCCGGAAGGUCAUCUCA |
| F805 | 1767 | AAAGAAAAGACAGUUGGAGGAAUCUGU | R805 | | |
| F806 | 1768 | GAAGAAAAUGAAAAGGAGUUAGCAGCAU | R806b | 1603 | CUCCUAGAGUUUUUCCAAGAACCAAGU |
| | | | R807 | 1604 | GAACCAAGUUCUUCCGAGGGAAU |
| F807 | 1769 | AGUGGCAAAAGAACUUCAGACUUUACA | | | |
| F808 | 1770 | GCGCUGCUCAGAAGCAAAA | | | |
| F809 | 1771 | GUAGAUCGCAUAAAGGAAGCAGUCA | R809 | 1605 | ACAGCGGCUGCGAUCACC |
| F810 | 1772 | GGAAGCAGUCAGGUCAAAGAAUAUGG | | | |
| F811 | | | R811 | 1606 | GCUGACUGCACAGGACAGG |
| F812 | 1773 | CAAGCAGAAACACUGUACAAAGAGAUU | | | |
| F813 | 1774 | GAGGGCGAGCUGCAUGAU | R813a | 1607 | CGAGACCCCAAAAGGUGUUUC |
| | | | R813b | 1608 | UCCACAUUUGUUGAGCACAAGGA |
| F814 | 1775 | CACAUCUUCAGGUGCUGGAUUUUUC | | | |
| F815 | 1776 | CUUUUGAAAAGCCAGUGAUGAUCUCAA | R815 | 1609 | CACCUUUAACUGCUUCAGGGUCAAUAU |
| F816 | 1777 | GCACCUUGACUUUAAGUGAGAGCA | R816 | 1610 | UGUUGUCCCGUGGCCAUU |
| F817 | 1778 | ACAGCACUGUUAUUACUACUUGGGUUUU | R817 | 1611 | GGCAUGAACCGUUCUGAGAUG |
| F818 | 1779 | CAAGCUCCUUACAUACCCAGCA | R818 | 1612 | CCAAAUUCGCCUUCUCCUAGAGU |
| F819 | 1780 | GCGUUUCCUCGCUUGCAUU | R819 | 1613 | CUCCUCUGCACCAAGGUAAACA |
| F820 | 1781 | CGGGCAGGAATCTGATGACTTT | R820 | 1614 | UCCCUUCUAGUAAUUUGGGAAUGCC |
| F821 | 1782 | GCAGGGCAGCAACAUCUUUG | | | |
| F822 | 1783 | GGCUCCUGAGACCUUUGAUAACAUAAC | | | |
| F823 | 1784 | CGUGUGCUCCCUGGAUAUUCUUAGUA | R823 | 1615 | UCAGCUUUCUCCCACUGUAUUGAAUUUU |
| F824 | 1785 | CUGGCUCCGGGUGACAGC | R824 | 1616 | UCGGAAGGGCUGUGGAAUUG |
| | | | R824b | 1617 | CGUAGGCACACUCAAACAACGA |
| F825 | 1747 | CUGGCUCCGGGUGACAGC | R825 | 1618 | CUGAUUUCUGAACAUGGACUGUGG |
| F826 | 1748 | GACUCCCAUGACCCCCAUC | R826 | 1619 | ACGAAGUGCAAUGGUCUUUAGGU |
| F827 | 1749 | AAAAAUGUUAUGUCAGCGUUUGGCUUAA | | | |
| F828 | 1750 | GUAGGCGCGAGCUAAGCA | R828 | 1620 | GUGAGUCAUUUGUCUUGCUUUUGGU |
| F829 | 1751 | CAGGUCAUAUUGAACAUUCCAGAUACCU | | | |
| F830 | 1752 | GGUCCUGACGCAGGCUUC | | | |
| F829 | 1621 | GACAGUCUGAAUCAUGUCCUUCAGU | | | |
| F830 | 1622 | GGGCUGCCCACCAUCUUC | | | |
| F831 | 1623 | UCAGCCUGAUAGUCUGGUACAAACU | | | |
| F831 | 1753 | GUACCUGCAUCAACCCCUCUAA | | | |
| F832 | 1754 | CAGAGACCCGUGCUGAGUUU | | | |
| F833 | 1755 | GGAGAGAAGAGUGCACAAUACCA | R833 | 1624 | CCUCCACCUUGGGCUACUCA |
| F834 | 1756 | CCUGUAAUCCCUGCACUUUAGGA | R834 | 1625 | GGGUGAGCCUUGACACACA |
| F835 | 1757 | ACUUUCCAGUUGAGCAUCCCAAAUU | R835 | 1626 | CAGGGAUCAGUUCAGCUGUACC |
| F836 | 1758 | CGUCAGCGUGAUAUGUACCGUAUUUUAU | | | |
| F837 | 1759 | CACCUCAGUAAUAUGGAAGUCCAAGUU | | | |

TABLE E

| Primer | | |
|---|---|---|
| Name | SEQ ID | Primer Sequence |
| F838 | 1627 | UGGGCUCUGUAAAGAAUAGUG |
| F839 | 1628 | UGCACACUUGGACAGCAUUUC |
| F840 | 1629 | CCAGGACCAAUCUGGUCACAAACAUA |
| F841 | 1630 | GGUGGGAGGAAAAGACAUAGGAT |
| F842 | 1631 | CUCCAGAGAGAAAGAAUCAACAGG |
| F843 | 1632 | GCAUCCGUGACUCUCUGGAC |
| F844 | 1633 | UCAGUGAGCCAAUUCCUUGUAAUAACUC |
| F845 | 1634 | CAGAUCCCAAGCUCUUCCUCUT |
| F846 | 1635 | GUUCAUGCCACUGCACUUCACT |
| F847 | 1636 | GGUGCACCCAUUACCCGAAT |
| F848 | 1637 | UCCCCAUAUAAGUUCAAGCCUGUGT |
| F849 | 1638 | UUGUAUAGCUACAGUUUUUCUGUUGGT |
| F850 | 1639 | UAAAUAUGUGAGUCAAUUCCCCAAGUG |
| F851 | 1640 | GGCUAGAUUUUCCCCGAUGAUAGUAGT |
| F852 | 1641 | GGCUAGAUUUUCCCCUAUGAUAGUAGT |
| F853 | 1642 | CAGUAAGUUAAAGGAUUGCAGGAG |
| F854 | 1643 | UGUGUAUAUGCAUUUACCUGUGAGUAUG |
| F855 | 1644 | UGUAACAAGGGCUACAGGAAUCAT |
| F856 | 1645 | GGGCAUCUCUUAUACUCAUGAAAUCAA |
| F857 | 1646 | CUAUGCAGAAGAAUGAACCAGGGAT |
| F858 | 1647 | UGAUUCAUUUCCAUAGGGUAAGUGAAAA |
| F859 | 1648 | GACAUUAUCACCAAUUUUUCUAGACG |
| F860 | 1649 | GACAUUCUCACCAAUUUUUCUAGACG |
| F861 | 1650 | UGUGACAAGGGUGAUUUUCCUC |
| F862 | 1651 | CAUAAUUGUAUGAGCCACUUCCCAT |
| F863 | 1652 | AGACUCACAAUGUACAAAAGCCUAUT |
| F864 | 1653 | AAUAUAUAUAAAGGGUAUGAUAGAACACUUGUC |
| F865 | 1654 | GGCCUGGCAACUUAUAUGUAUUUUUGUAUT |
| F866 | 1655 | GGCCUGACAACUUAUAUGUAUUUUUGUAUT |
| F867 | 1656 | CCAUCCUUAUCUCUUGUGUAUCUAUUCAUUCAA |
| F868 | 1657 | GAUUUGUCUGUAAUUGCCAGCAAAA |
| F869 | 1658 | GAGCAAGACACCAUCUCAAGAA |
| F870 | 1659 | CAUGAUUGAUACAUGGAAAGAAUUCUCT |
| F871 | 1660 | ACCCAAAUCAACUCAACUCCAGUG |
| F872 | 1661 | UUAGAGCAUUUAAAGUAAGCCACAGUGT |
| F873 | 1662 | CUGUACACAGGGCUUCCGAGT |
| F874 | 1663 | UUUCAGGGCUGUGAUCACUAGCAC |
| F875 | 1664 | AGAUACAUAGGUUAGAUAGAGAUAGGACAGA |
| R838a | 1665 | AUCAGAGCUUAAACUGGGAAG |
| R838b | 1666 | AUCAGAGCUUAAACUGGGAAA |
| R839 | 1667 | GUCUCAGUUUUCCUACCUGUAAAAUGAAG |
| R840 | 1668 | ACUUAUUCUGACAGUUCUCUUUUUCCCT |
| R841 | 1669 | GGUGGCAGUGAGCUGUAACAGUA |
| R842 | 1670 | UCAGCCUCCAUAUCACUUGAGC |
| R843 | 1671 | AACUUGGGUUGAGCCAUAGGC |
| R844 | 1672 | CCUGGUUCCAUGGAUUCCACAUUAAGA |
| R845 | 1673 | GCGUUUGUGUGUGCAUCUGT |
| R846 | 1674 | UCUGGUGUGUGGAGAUGUCUUAC |
| R847a | 1675 | GGCUGCAAAAAGCUAUAAUUGUACC |
| R847b | 1676 | GGCUGCAAAAAGCUAUAACUGUACC |
| R848 | 1677 | UGUGUUAGUCAGGAUUCUUCAGAGA |
| R849 | 1678 | UUCAGUUAUAUGUGUAUAAAUGUGUGCAUUG |
| R850 | 1679 | CUCCAGAGACAGACUAAUAGGAGGUA |
| R851 | 1680 | CCUGUGCCCAAGUUGAGAGAAT |
| R853 | 1681 | UAAUCCAGCUGUGGGAGGGA |
| R854 | 1682 | GGUGCUAGGUGUGCUCAGGA |
| R855 | 1683 | CUUCACUCUCCUUCCCAAAUGUUUAUG |
| R856 | 1684 | CUAUGAUUCCCCCACUGCAGUC |
| R857 | 1685 | AGACCCCAAAAUUACUUGAGCCAAUUUA |
| R858 | 1686 | ACUUCAACUUCAAUUCAUCCACUGAAA |
| R859 | 1687 | UGCUUGCCUGUAUGAAAAUAUCUC |
| R861 | 1688 | UCCAAUCAUAGCCACAGUUUACAA |
| R862 | 1689 | GCACUCUUAUUCAUCUAGUUGCCUGT |
| R863a | 1690 | CAUCAUGUGAGCCAAUUCCUCUC |
| R863b | 1691 | CAUCAUGUGAGCCAAGUCCUCUC |
| R864a | 1692 | UUGCACCAAAUAUUGGUAAUUAAAUGUUUACT |
| R864b | 1693 | UUGCACCACAUAUUGGUAAUUAAAUGUUUACT |
| R865 | 1694 | CACUGUAUCGUAUCCCAUUGCG |
| R867 | 1695 | UUGCAAGCAAUUGCCAUAGAGGGA |
| R868 | 1696 | ACAGAUUAAACUGUAACCAAAAUAAAAUUAGGC |
| R869a | 1697 | UGCCUAACCUAUGGUCAUAACG |
| R869b | 1698 | UGCCUAACCUAUGGUCAUACCG |
| R870 | 1699 | CCCAGGAGGUGGAGAUUGAA |
| R871 | 1700 | UCCAUGUACUUUGUCCAAUGCUGA |
| R872 | 1701 | UGUCAACACGAUUAACAUGCAAAGA |
| R873 | 1702 | CAAAAUUCAAAGGGUAUCUGGGCUCT |
| R874 | 1703 | UGUGCGCUGGUCUUACUCCUGUT |
| R875 | 1704 | GCCCUAGUGGAUGAUAAGAAUAAUCAG |

1. A method for preparing a library of target nucleic acid sequences comprising
(a) contacting a nucleic acid sample with a plurality of adaptors capable of amplification of one or more target nucleic acid sequences in the sample under conditions wherein the target nucleic acid(s) undergo a first amplification;
(b) digesting resulting first amplification products to reduce or eliminate resulting primer dimers and prepare partially digested target amplicons, producing gapped, double stranded amplicons, then repairing the partially digested target amplicons; and
(c) amplifying the repaired target amplicons in a second amplification using universal primers, thereby producing a library of target nucleic acid sequences;
wherein each of the plurality of adaptors comprises a universal handle sequence and a target nucleic acid sequence and a cleavable moiety and optionally one or more tag sequences; and wherein at least two and up to one hundred thousand target specific adaptor pairs are included; and
wherein the target nucleic acid sequence of the adaptor includes at least one cleavable moiety and the universal handle sequence does not include the cleavable moiety.

2. The method of claim 1 wherein an optional tag sequence is included in at least one adaptor, and the cleavable moieties are included in the adaptor sequence flanking either end of the tag sequence.

3. The method of claim 1 carried out in a single, addition only workflow reaction, allowing for rapid production of highly multiplexed targeted libraries.

4. The method of claim 2 wherein all of the adaptors comprise tag sequences having cleavable groups flanking either end of the tag sequences.

5. The method of claim 1 wherein the digestion and repair of step (b) is carried out in a single step or is carried out in a temporally separate manner at different temperatures.

6. The method of claim 1 wherein one or more of the method steps is conducted in manual mode or in an automated mode or a combination thereof.

7. The method of claim 1 further comprising at least one purification step.

8. The method of claim 7 wherein a purification step is carried out only after step (c) or wherein a first purification step is carried out after step (b) and a second purification step is carried out after step (c).

9. The method of claim 2 wherein each target specific pair of the plurality of adaptors includes up to 16,777,216 different adaptor combinations comprising different tag sequences.

10. The method of claim 1, further comprising analyzing the sequence of the resulting library of target nucleic acid sequences.

11. The method of claim 10 wherein analyzing comprises sequencing by traditional sequencing reactions, high throughput next generation sequencing, targeted multiplex array sequence detection, or any combination of two or more of the foregoing.

12. The method of claim 10 further comprising determining the abundance of at least one of the target nucleic acid sequences in the sample.

13. The method of claim 11 wherein sequencing is carried out in a bidirectional manner, thereby generating sequence reads in both forward and reverse strands for any given amplicon.

14. The method of claim 1, wherein a digestion reagent comprises any one or a combination of uracil DNA glycosylase (UDG), apurinic endonuclease (e.g., APE1), RecJf, formamidopyrimidine [fapy]-DNA glycosylase (fpg), Nth endonuclease III, endonuclease VIII, polynucleotide kinase (PNK), Taq DNA polymerase, DNA polymerase I and/or human DNA polymerase beta.

15. The method of claim 1 wherein the repair reagent comprises any one or a combination of Phusion DNA polymerase, Phusion U DNA polymerase, SuperFi DNA polymerase, Taq DNA polymerase, Human DNA polymerase beta, T4 DNA polymerase and/or T7 DNA polymerase, SuperFiU DNA polymerase, E. coli DNA ligase, T3 DNA ligase, T4 DNA ligase, T7 DNA ligase, Taq DNA ligase, and/or 9° N DNA ligase.

16. The method of claim 5, wherein the plurality of gapped polynucleotide products in step (b) are contacted with the digestion and repair reagents simultaneously, or the plurality of gapped polynucleotide products in step (b) are contacted sequentially with the digestion and repair reagents.

17. A composition comprising a nucleic acid library generated by step (a) of claim 1.

18. A method for preparing a library of target nucleic acid sequences comprising
(a) contacting a nucleic acid sample with a plurality of adaptors capable of amplification of one or more target nucleic acid sequences in the sample under conditions wherein the target nucleic acid(s) undergo a first amplification;
(b) digesting resulting first amplification products to reduce or eliminate resulting primer dimers and prepare partially digested target amplicons, producing gapped, double stranded amplicons, then repairing the partially digested target amplicons; and
(c) amplifying the repaired target amplicons in a second amplification using universal primers, thereby producing a library of target nucleic acid sequences;
wherein each of the plurality of adaptors comprise a universal handle sequence, one or more tag sequences, a target nucleic acid sequence and a cleavable moiety; and wherein at least two and up to one hundred thousand target specific adaptor pairs are included;
wherein the target nucleic acid sequence of the adaptor includes at least one cleavable moiety, cleavable moieties are included in the flanking either end of the tag sequence and the universal handle sequence does not include the cleavable moiety.

19. The method of claim 1, wherein the second amplification utilizes at least two primer pairs to incorporate two universal sequences on each end of the target amplicons to enable downstream bi-directional sequencing.

20. The method of claim 18, wherein the second amplification utilizes at least two primer pairs to incorporate two universal sequences on each end of the target amplicons to enable downstream bi-directional sequencing.
